(19) United States
(12) Patent Application Publication
(10) Pub. No.: US 2011/0183382 A1
(43) Pub. Date: Jul. 28, 2011

(54) METHODS AND COMPOSITIONS FOR PRODUCING CHEMICAL PRODUCTS FROM C. PHYTOFERMENTANS

(75) Inventors: Matthias Schmalisch, Marlborough, MA (US); Chelsea Ju, Natick, MA (US); Francis H. Verhoff, Cincinnati, OH (US); Gregory S. Coil, Northborough, MA (US)

(73) Assignee: Qteros, Inc., Marlborough, MA (US)

(21) Appl. No.: 12/969,582

(22) Filed: Dec. 15, 2010

Related U.S. Application Data
(60) Provisional application No. 61/286,729, filed on Dec. 15, 2009.

(51) Int. Cl. C12P 13/20; C12N 1/00; C12N 1/21; C12P 13/14; C12P 7/46; C12P 17/04; C12P 7/52; C12P 7/58; C12P 7/40; C12P 7/20; C12P 7/18; C12P 5/02; C12P 7/14; C12P 7/04; C12P 7/16; C12P 3/00; C12P 7/56; C12P 7/54; C12M 1/00

(52) U.S. Cl. 435/109; 435/243; 435/252.3; 435/110; 435/145; 435/126; 435/141; 435/137; 435/136; 435/159; 435/158; 435/167; 435/162; 435/157; 435/160; 435/168; 435/139; 435/140; 435/303.1

(57) ABSTRACT

This invention provides systems and methods for the production of compounds by C. phytofermentans. C. phytofermentans is genetically-engineered for hydrolysis and fermentation of carbonaceous biomass to synthesize compounds of commercial value.

### CLAIM OF PRIORITY

This application claims the benefit of priority from U.S. Provisional Patent Application Ser. No. 61/286,729, filed on Dec. 15, 2009, the entire contents of which are incorporated by reference herein.

### BACKGROUND OF THE INVENTION

Biomass is a renewable source of energy, which can be biologically fermented to produce an end-product such as an organic acid or other useful compound. There is a growing consensus that fermenting chemicals from renewable resources such as cellulosic and lignocellulosic plant materials has great potential and can replace chemical synthesis that use petroleum reserves as energy sources. Fermentation by microbes can reduce greenhouse gases and support agriculture. However, microbial fermentation requires adapting strains of microbes to particular feedstocks and fermentation media. Because certain microbial species are particular to the products they synthesize, different microbes have to be adapted to a process to make more than one product.

Clostridia are well known as natural synthesizers of chemical products. However, many of the clostridial species can only ferment biomass to a few specific products and most of these end products are produced in low amounts. Although it is ecologically desirable to develop renewable organic substances, it is not yet economically feasible. There remains a strong need for one microbial species that can produce many different chemicals, utilize different feedstocks, and through modification, ferment biomass cost-effectively for specific chemicals.

### SUMMARY OF THE INVENTION

Methods and compositions described herein include a composition for production of fermentation end-products comprising: a carbonaceous biomass, and an organism that is capable of direct hydrolysis and fermentation of the biomass, wherein the organism is modified to produce a higher concentration of the compound compared to the naturally-occurring organism.

In one aspect of the invention, a composition is provided for producing first and second fermentation end-products comprising: a carbonaceous biomass, a microorganism that hydrolyses and ferments pentose and hexose saccharides from the biomass, a first fermentation end-product, wherein the fermentation end-product is not ethanol, propanol, isopropanol, n-butanol, hydrogen, formic acid, lactic acid, acetic acid, formate, lactate or acetate, and a second fermentation end-product. In one embodiment, the first fermentation end-product is aspartic acid, aspartate, glutamic acid, glutamate, malic acid, or malate. In another embodiment, the second fermentation end-product is not ethanol, propanol, isopropanol, n-butanol, hydrogen, formic acid, lactic acid acetic acid, formate, lactate or acetate. In one embodiment, the microorganism is a bacterium. In another embodiment, the microorganism is a species of Clostridia. In another embodiment, the microorganism is Clostridium phytofermentans. In another embodiment, the microorganism is Clostridium sp. Q.D. In another embodiment, the microorganism is non-recombinant. In another embodiment, the microorganism is recombinant. In another embodiment, the microorganism is genetically modified or mutagenized to enhance production of a fermentation end-product. In another embodiment, the microorganism is genetically modified to express a protein encoded by a heterologous polynucleotide. In another embodiment, the microorganism is genetically modified to enhance expression of a protein by deregulation of an endogenous promoter or by expression of an additional copy of an endogenous polynucleotide encoding the protein. In another embodiment, the non-genetically modified or mutagenized strain of the microorganism cannot produce the fermentation end-product. In another embodiment, the first fermentation end-product is not ethanol, propanol, isopropanol, n-butanol, hydrogen, formic acid, lactic acid acetic acid, formate, lactate or acetate. In another embodiment, the first fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene. In another embodiment, the second fermentation end-product is ethanol, propanol, isopropanol, n-butanol, hydrogen, formic acid, lactic acid acetic acid, formate, lactate or acetate and is present at a lower amount than the first fermentation product. In another embodiment, the second fermentation end-product is ethanol, propanol, isopropanol, n-butanol, hydrogen, formic acid, lactic acid acetic acid, formate, lactate or acetate and is present at an amount less than 99% of the amount of the first fermentation product. In another embodiment, the second fermentation end-product is ethanol, propanol, isopropanol, n-butanol, hydrogen, formic acid, lactic acid acetic acid, formate, lactate or acetate and is present at an amount less than 75% of the amount the of the first fermentation product. In another embodiment, the second fermentation end-product is ethanol, propanol, isopropanol, n-butanol, hydrogen, formic acid, lactic acid acetic acid, formate, lactate or acetate and is present at an amount less than 50% of the amount of the first fermentation product. In another embodiment, the second fermentation end-product is ethanol, propanol, isopropanol, n-butanol, hydrogen, formic acid, lactic acid acetic acid, formate, lactate or acetate and is present at an amount less than 30% of the amount of the first fermentation product. In another embodiment, the second fermentation end-product is ethanol, propanol, isopropanol, n-butanol, hydrogen, formic acid, lactic acid acetic acid, formate, lactate or acetate and is present at an amount less than 20% of the amount of the first fermentation product. In another embodiment, the second fermentation end-product is ethanol, propanol, isopropanol, n-butanol, hydrogen, formic acid, lactic acid acetic acid, formate, lactate or acetate and is present at an amount less than 10% of the amount of the first fermentation product. In another embodiment, the second fermentation end-product is ethanol, propanol, isopropanol, n-butanol, hydrogen, formic acid, lactic acid acetic acid, formate, lactate or acetate and is present at an amount less than 5% of the amount of the first fermentation product. In another embodiment, the first fermentation end-product is present in an amount greater than 0.1-100% (such as 0.1-99, 1-99, 1-95, 1-90, 1-80, 1-75, 1-70, 1-65, 1-60, 1-55, 1-50, 1-45, 1-40, 1-35, 1-30, 1-25, 1-20, 1-15, 1-10, 10-20, 20-30, 30-40, 40-50, 50-60, 60-70, 70-80, 80-90, 50-80, 40-60, 30-70, 20-80, or 10-90%) of the amount of the second fermentation product. In another embodiment, the first fermentation end-product is present in an amount greater than 50% (such as 2, 3, 4, 5, 6, 7, 8, 9 or 10 times) the amount of the second fermentation product. In another embodiment, the biomass comprises one or more of xylan, cellulose, hemicellulose, fructose, glucose, mannose, rhamnose, or xylose. In another embodiment, the biomass is plant matter. In another embodiment, the biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae. In another embodiment, the biomass comprises a higher concentration of oligomeric carbohydrates relative to monomeric carbohydrates. In another embodiment, the hydrolysis provides results in a greater concentration of cellobiose relative to monomeric carbohydrates. In another embodiment, the monomeric carbohydrates comprise xylose and arabinose. In another embodiment, the biomass is pre-treated with an acid, alkali or heat prior to contact with the microorganism. In another embodiment, the composition further comprises a second species of microorganism. In another embodiment, the second species of microorganism is a yeast or bacterium. In another embodiment, the second species of microorganism is S. cerevisiae or Aspergillus niger.

In another aspect of the invention, a composition is provided a composition for producing a fermentation end-product comprising: a carbonaceous biomass, and a Clostridium sp. Q.D microorganism to produce the fermentation end-product. In one embodiment, the microorganism is non-recombinant. In another embodiment, the microorganism is recombinant. In another embodiment, the microorganism is genetically modified or mutagenized to enhance production of a fermentation end-product. In another embodiment, the microorganism is genetically modified to express a protein encoded by a heterologous polynucleotide. In another embodiment, the microorganism is genetically modified to enhance expression of a protein by deregulation of an endogenous promoter or by expression of an additional copy of an endogenous polynucleotide encoding the protein. In another embodiment, the non-genetically modified or mutagenized strain of the microorganism cannot produce the fermentation end-product. In another embodiment, the biomass comprises one or more of xylan, cellulose, hemicellulose, fructose, glucose, mannose, rhamnose, or xylose. In another embodiment, the fermentation end-product is aspartic acid, aspartate, glutamic acid, glutamate, malic acid, or malate. In another embodiment, the fermentation end-product is not ethanol, propanol, isopropanol, n-butanol, hydrogen, formic acid, lactic acid acetic acid, formate, lactate or acetate. In another embodiment, the fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene. In another embodiment, the biomass is plant matter. In another embodiment, the biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae. In another embodiment, the biomass comprises a higher concentration of oligomeric carbohydrates relative to monomeric carbohydrates. In another embodiment, the hydrolysis provides results in a greater concentration of cellobiose relative to monomeric carbohydrates. In another embodiment, the monomeric carbohydrates comprise xylose and arabinose. In another embodiment, the biomass is pre-treated with an acid, alkali or heat prior to contact with the microorganism. In another embodiment, the composition further comprises a second species of microorganism. In another embodiment, the second species of microorganism is a yeast or bacterium. In another embodiment, the second species of microorganism is S. cerevisiae or Aspergillus niger.

In another aspect of the invention, a composition is provided for producing a fermentation end-product comprising: a carbonaceous biomass, and a microorganism that hydrolyses and ferments pentose and hexose saccharides from the biomass, wherein the microorganism is genetically modified or mutagenized to produce a higher concentration of the fermentation end-product compared to a non-genetically modified form of the microorganism. In one embodiment, the microorganism is a bacterium. In another embodiment, the microorganism is a species of Clostridia. In another embodiment, the microorganism is Clostridium phytofermentans. In another embodiment, the microorganism is Clostridium sp. Q.D. In another embodiment, the microorganism is non-recombinant. In another embodiment, the microorganism is recombinant. In another embodiment, the microorganism is genetically modified or mutagenized to enhance production of a fermentation end-product. In another embodiment, the microorganism is genetically modified to express a protein encoded by a heterologous polynucleotide. In another embodiment, the microorganism is genetically modified to enhance expression of a protein by deregulation of an endogenous promoter or by expression of an additional copy of an endogenous polynucleotide encoding the protein. In another embodiment, the non-genetically modified or mutagenized strain of the microorganism cannot produce the fermentation end-product. In another embodiment, the fermentation end-product is aspartic acid, aspartate, glutamic acid, glutamate, malic acid, or malate. In another embodiment, the fermentation end-product is not ethanol, propanol, isopropanol, n-butanol, hydrogen, formic acid, lactic acid acetic acid, formate, lactate or acetate. In another embodiment, the fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene. In another embodiment, the biomass comprises one or more of xylan, cellulose, hemicellulose, fructose, glucose, mannose, rhamnose, or xylose. In another embodiment, the biomass is plant matter. In another embodiment, the biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae. In another embodiment, the biomass comprises a higher concentration of oligomeric carbohydrates relative to monomeric carbohydrates. In another embodiment, the hydrolysis provides results in a greater concentration of cellobiose relative to monomeric carbohydrates. In another embodiment, the monomeric carbohydrates comprise xylose and arabinose. In another embodiment, the biomass is pre-treated with an acid, alkali or heat prior to contact with the microorganism. In another embodiment, the composition further comprises a second species of microorganism. In another embodiment, the second species of microorganism is a yeast or bacterium. In another embodiment, the second species of microorganism is S. cerevisiae or Aspergillus niger.

In another aspect of the invention, a composition is provided producing a fermentation end-product comprising: a carbonaceous biomass, and a genetically modified or mutagenized microorganism that hydrolyses and ferments pentose and hexose saccharides from the biomass, wherein a non genetically modified or non-mutagenized strain of the microorganism is genetically modified or mutagenized to produce the fermentation end-product. In one embodiment, the microorganism is a bacterium. In another embodiment, the microorganism is a species of Clostridia. In another embodiment, the microorganism is Clostridium phytofermentans. In another embodiment, the microorganism is Clostridium sp. Q.D. In another embodiment, the microorganism is non-recombinant. In another embodiment, the microorganism is recombinant. In another embodiment, the microorganism is genetically modified or mutagenized to enhance production of a fermentation end-product. In another embodiment, the microorganism is genetically modified to express a protein encoded by a heterologous polynucleotide. In another embodiment, the microorganism is genetically modified to enhance expression of a protein by deregulation of an endogenous promoter or by expression of an additional copy of an endogenous polynucleotide encoding the protein. In another embodiment, the non-genetically modified or mutagenized strain of the microorganism cannot produce the fermentation end-product. In another embodiment, the first fermentation end-product is aspartic acid, aspartate, glutamic acid, glutamate, malic acid, or malate. In another embodiment, the fermentation end-product is not ethanol, propanol, isopropanol, n-butanol, hydrogen, formic acid, lactic acid acetic acid, formate, lactate or acetate. In another embodiment, the fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene. In another embodiment, the biomass comprises one or more of xylan, cellulose, hemicellulose, fructose, glucose, mannose, rhamnose, or xylose. In another embodiment, the biomass is plant matter. In another embodiment, the biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae. In another embodiment, the biomass comprises a higher concentration of oligomeric carbohydrates relative to monomeric carbohydrates. In another embodiment, the hydrolysis provides results in a greater concentration of cellobiose relative to monomeric carbohydrates. In another embodiment, the monomeric carbohydrates comprise xylose and arabinose. In another embodiment, the biomass is pre-treated with an acid, alkali or heat prior to contact with the microorganism. In another embodiment, the composition further comprises a second species of microorganism. In another embodiment, the second species of microorganism is a yeast or bacterium. In another embodiment, the second species of microorganism is S. cerevisiae or Aspergillus niger.

In another aspect of the invention, a fermentation composition is provided comprising a carbonaceous biomass, a strain of Clostridium phytofermentans, and aspartic acid, aspartate, glutamic acid, glutamate, malic acid, or malate.

In another aspect of the invention, a fermentation composition is provided comprising a carbonaceous biomass, Clostridium sp. Q.D, and aspartic acid, aspartate, glutamic acid, glutamate, malic acid, or malate.

In another aspect of the invention, a process is provided for producing a first and second fermentation end-product comprising contacting a carbonaceous biomass with: a microorganism that hydrolyses and ferments pentose and hexose saccharides from the biomass to produce the first and second fermentation end-products, wherein the first fermentation end-product is not ethanol, propanol, isopropanol, n-butanol, hydrogen, formic acid, lactic acid acetic acid, formate, lactate or acetate, and allowing sufficient time for the hydrolysis and fermentation to produce the first and second fermentation end-products.

In one embodiment, the microorganism is a bacterium. In another embodiment, the microorganism is a species of Clostridia. In another embodiment, the microorganism is Clostridium phytofermentans. In another embodiment, the microorganism is Clostridium sp. Q.D. In another embodiment, the microorganism is non-recombinant. In another embodiment, the microorganism is recombinant or a mutant. In another embodiment, the microorganism is genetically modified or mutagenized to enhance production of a fermentation end-product. In another embodiment, the microorganism is genetically modified to express a protein encoded by a heterologous polynucleotide. In another embodiment, the microorganism is genetically modified to enhance expression of an endogenous protein by deregulation of an endogenous promoter or by expressing an additional copy of an endogenous polynucleotide encoding the protein. In another embodiment, the organism comprises one or more heterologous or exogenous polynucleotides that enhance the yield of fermentation end-products. In another embodiment, the first fermentation end-product is aspartic acid, aspartate, glutamic acid, glutamate, malic acid, or malate. In another embodiment, the fermentation end-product is aspartic acid, aspartate, glutamic acid, glutamate, malic acid, or malate. In another embodiment, the first fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene. In another embodiment, the fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene. In another embodiment, the fermentation end-product is not ethanol, propanol, isopropanol, n-butanol, hydrogen, formic acid, lactic acid acetic acid, formate, lactate or acetate. In one embodiment, the second fermentation end-product is not ethanol, propanol, isopropanol, n-butanol, hydrogen, formic acid, lactic acid acetic acid, formate, lactate or acetate. In another embodiment, the second fermentation end-product is ethanol, propanol, isopropanol, n-butanol, hydrogen, formic acid, lactic acid acetic acid, formate, lactate or acetate and is present at a lower amount than the first fermentation product. In another embodiment, the second fermentation end-product is ethanol, propanol, isopropanol, n-butanol, hydrogen, formic acid, lactic acid acetic acid, formate, lactate or acetate and is present at an amount less than 99% of the amount of the first fermentation product. In another embodiment, the second fermentation end-product is ethanol, propanol, isopropanol, n-butanol, hydrogen, formic acid, lactic acid acetic acid, formate, lactate or acetate and is present at an amount less than 75% of the amount of the first fermentation product. In another embodiment, the second fermentation end-product is ethanol, propanol, isopropanol, n-butanol, hydrogen, formic acid, lactic acid acetic acid, formate, lactate or acetate and is present at an amount less than 50% of the amount of the first fermentation product. In another embodiment, the second fermentation end-product is ethanol, propanol, isopropanol, n-butanol, hydrogen, formic acid, lactic acid acetic acid, formate, lactate or acetate and is present at an amount less than 30% of the amount of the first fermentation product. In another embodiment, the second fermentation end-product is ethanol, propanol, isopropanol, n-butanol, hydrogen, formic acid, lactic acid acetic acid, formate, lactate or acetate and is present at an amount less than 20% of the amount of the first fermentation product. In another embodiment, the second fermentation end-product is ethanol, propanol, isopropanol, n-butanol, hydrogen, formic acid, lactic acid acetic acid, formate, lactate or acetate and is present at an amount less than 10% of the amount of the first fermentation product. In another embodiment, the second fermentation end-product is ethanol, propanol, isopropanol, n-butanol, hydrogen, formic acid, lactic acid acetic acid, formate, lactate or acetate and is present at an amount less than 5% of the amount of the first fermentation product. In another embodiment, the contact is in a large-scale fermentation vessel, wherein the fermentation vessel is adapted to provide suitable conditions for fermentation of one or more carbohydrate into a fermentation end-product.

In another embodiment, the biomass comprises one or more of xylan, cellulose, hemicellulose, fructose, glucose, mannose, rhamnose, or xylose. In one embodiment, the biomass is plant matter. In another embodiment, the biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae. In another embodiment, the biomass comprises a higher concentration of oligomeric carbohydrates relative to monomeric carbohydrates. In another embodiment, the hydrolysis provides results in a greater concentration of cellobiose relative to monomeric carbohydrates. In another embodiment, the monomeric carbohydrates comprise xylose and arabinose. In o another ne embodiment, the biomass is pre-treated with an acid, alkali or heat prior to contact with the microorganism. In another embodiment, the process further comprises a second species of microorganism. In another embodiment, the second species of microorganism is a yeast or a bacterium. In another embodiment, the second species of microorganism is S. cerevisiae or Aspergillus niger. In another embodiment, a first fermentation end-product is produced by the process of the invention. In another embodiment, the fermentation end-product is not ethanol, propanol, isopropanol, n-butanol, hydrogen, formic acid, lactic acid acetic acid, formate, lactate or acetate.

In another aspect of the invention, a process is provided for producing a fermentation end-product comprising contacting a carbonaceous biomass with: microorganism that hydrolyses and ferments pentose and hexose saccharides from the biomass, wherein the microorganism is genetically modified or mutagenized to produce a higher concentration of the fermentation end-product compared to a non-genetically modified or non-mutagenized form of the microorganism, and allowing sufficient time for the hydrolysis and fermentation to produce the fermentation end-product.

In one embodiment, the microorganism is a bacterium. In another embodiment, the microorganism is a species of Clostridia. In another embodiment, the microorganism is Clostridium phytofermentans. In another embodiment, the microorganism is Clostridium sp. Q.D. In another embodiment, the microorganism is genetically modified or mutagenized to enhance production of a fermentation end-product. In another embodiment, the microorganism is genetically modified to express a protein encoded by a heterologous polynucleotide. In another embodiment, the microorganism is genetically modified to enhance expression of an endogenous protein by deregulation of an endogenous promoter or by expressing an additional copy of an endogenous polynucleotide encoding the protein. In another embodiment, the organism comprises one or more heterologous or exogenous polynucleotides that enhance the yield of fermentation end-products. In another embodiment, the first fermentation end-product is aspartic acid, aspartate, glutamic acid, glutamate, malic acid, or malate. In another embodiment, the fermentation end-product is aspartic acid, aspartate, glutamic acid, glutamate, malic acid, or malate. In another embodiment, the first fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene. In another embodiment, the fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene. In another embodiment, the fermentation end-product is not ethanol, propanol, isopropanol, n-butanol, hydrogen, formic acid, lactic acid acetic acid, formate, lactate or acetate. In one embodiment, the second fermentation end-product is not ethanol, propanol, isopropanol, n-butanol, hydrogen, formic acid, lactic acid acetic acid, formate, lactate or acetate.

In another embodiment, the contact is in a large-scale fermentation vessel, wherein the fermentation vessel is adapted to provide suitable conditions for fermentation of one or more carbohydrate into a fermentation end-product.

In another embodiment, the biomass comprises one or more of xylan, cellulose, hemicellulose, fructose, glucose, mannose, rhamnose, or xylose. In one embodiment, the biomass is plant matter. In another embodiment, the biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae. In another embodiment, the biomass comprises a higher concentration of oligomeric carbohydrates relative to monomeric carbohydrates. In another embodiment, the hydrolysis provides results in a greater concentration of cellobiose relative to monomeric carbohydrates. In another embodiment, the monomeric carbohydrates comprise xylose and arabinose. In o another ne embodiment, the biomass is pre-treated with an acid, alkali or heat prior to contact with the microorganism. In another embodiment, the process further comprises a second species of microorganism. In another embodiment, the second species of microorganism is a yeast or a bacterium. In another embodiment, the second species of microorganism is S. cerevisiae or Aspergillus niger. In another embodiment, a first fermentation end-product is produced by the process of the invention. In another embodiment, the fermentation end-product is not ethanol, propanol, isopropanol, n-butanol, hydrogen, formic acid, lactic acid acetic acid, formate, lactate or acetate.

In another aspect of the invention, a process is provided for producing a fermentation end-product comprising contacting a carbonaceous biomass with a microorganism that hydrolyses and ferments pentose and hexose saccharides from the biomass, wherein a non-genetically modified strain of the microorganism is genetically modified to produce the fermentation end-product and allowing sufficient time for the hydrolysis and fermentation to produce the fermentation end-product.

In one embodiment, the microorganism is a bacterium. In one embodiment, the microorganism is a species of Clostridia. In one embodiment, the microorganism is Clostridium phytofermentans. In one embodiment, the microorganism is recombinant or a mutant. In one embodiment, the microorganism is genetically modified or mutagenized to enhance production of a fermentation end-product. In one embodiment, the microorganism is genetically modified to express a protein encoded by a heterologous polynucleotide. In one embodiment, the microorganism is genetically modified to enhance expression of an endogenous protein by deregulation of an endogenous promoter or by expressing an additional copy of an endogenous polynucleotide encoding the protein. In one embodiment, the organism comprises one or more heterologous or exogenous polynucleotides that enhance the yield of fermentation end-products. In one embodiment, the first fermentation end-product is aspartic acid, aspartate, glutamic acid, glutamate, malic acid, or malate. In one embodiment, the fermentation end-product is aspartic acid, aspartate, glutamic acid, glutamate, malic acid, or malate. In one embodiment, the first fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene. In one embodiment, the fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene. In one embodiment, the fermentation end-product is not ethanol, propanol, isopropanol, n-butanol, hydrogen, formic acid, lactic acid acetic acid, formate, lactate or acetate. In one embodiment, the second fermentation end-product is not ethanol, propanol, isopropanol, n-butanol, hydrogen, formic acid, lactic acid acetic acid, formate, lactate or acetate.

In one embodiment, the contact is in a large-scale fermentation vessel, wherein the fermentation vessel is adapted to provide suitable conditions for fermentation of one or more carbohydrate into a fermentation end-product.

In one embodiment, the biomass comprises one or more of xylan, cellulose, hemicellulose, fructose, glucose, mannose, rhamnose, or xylose. In one embodiment, the biomass is plant matter. In one embodiment, the biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae. In one embodiment, the biomass comprises a higher concentration of oligomeric carbohydrates relative to monomeric carbohydrates. In one embodiment, the hydrolysis provides results in a greater concentration of cellobiose relative to monomeric carbohydrates. In one embodiment, the monomeric carbohydrates comprise xylose and arabinose. In one embodiment, the biomass is pre-treated with an acid, alkali or heat prior to contact with the microorganism. In one embodiment, the process further comprises a second species of microorganism. In one embodiment, the second species of microorganism is a yeast or a bacterium. In one embodiment, the second species of microorganism is S. cerevisiae or Aspergillus niger. In one embodiment, a the fermentation end-product is produced by the process of the invention. In one embodiment, the fermentation end-product is not ethanol, propanol, isopropanol, n-butanol, hydrogen, formic acid, lactic acid acetic acid, formate, lactate or acetate.

In another aspect of the invention, a process is provided for producing a fermentation end-product comprising contacting a carbonaceous biomass with a Clostridium sp. Q.D, microorganism to produce the fermentation end-product, and allowing sufficient time for the hydrolysis and fermentation to produce the fermentation end-product. In one embodiment, the Clostridium sp. Q.D, microorganism is non-recombinant. In one embodiment, the Clostridium sp. Q.D, microorganism is recombinant. In one embodiment, the Clostridium sp. Q.D, microorganism is genetically modified or mutagenized to enhance production of a fermentation end-product. In one embodiment, the Clostridium sp. Q.D, microorganism comprises one or more heterologous or exogenous polynucleotides that enhance the yield of fermentation end-products. In one embodiment, the fermentation end-product is aspartic acid, aspartate, glutamic acid, glutamate, malic acid, or malate. In one embodiment, the fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene. In one embodiment, the contact is in a large-scale fermentation vessel, wherein the fermentation vessel is adapted to provide suitable conditions for fermentation of one or more carbohydrate into a fermentation end-product.

In one embodiment, the biomass comprises one or more of xylan, cellulose, hemicellulose, fructose, glucose, mannose, rhamnose, or xylose. In one embodiment, the biomass comprises wherein the carbonaceous biomass is plant matter. In one embodiment, the biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae.

In one embodiment, the biomass comprises a higher concentration of oligomeric carbohydrates relative to monomeric carbohydrates. In one embodiment, the hydrolysis provides results in a greater concentration of cellobiose relative to monomeric carbohydrates. In one embodiment, the monomeric carbohydrates comprise xylose and arabinose. In one embodiment, the biomass is pre-treated with an acid, alkali or heat prior to contact with the microorganism. In one embodiment, the process further comprises a second species of microorganism. In one embodiment, the second species of microorganism is a yeast. In one embodiment, the second species of microorganism is S. cerevisiae or Aspergillus niger.

In one embodiment, a fermentation end-product is produced by the process described herein. In one embodiment, a fermentation end-product is produced by the process described herein, wherein the fermentation end-product is not ethanol, propanol, isopropanol, n-butanol, hydrogen, formic acid, lactic acid acetic acid, formate, lactate or acetate.

In one aspect of the invention, a process is provided for producing a fermentation end-product that is aspartic acid, aspartate, glutamic acid, glutamate, malic acid, or malate, comprising contacting a carbonaceous biomass with a strain of Clostridium phytofermentans and allowing sufficient time for hydrolysis and fermentation of the carbonaceous biomass by Clostridium phytofermentans to produce a fermentation end-product that is the aspartic acid, aspartate, glutamic acid, glutamate, malic acid, or malate. In one embodiment, the fermentation end-product is aspartic acid, aspartate, glutamic acid, glutamate, malic acid, or malate.

In another aspect of the invention, a process is provided for producing a fermentation end-product that is aspartic acid, aspartate, glutamic acid, glutamate, malic acid, or malate, comprising contacting a carbonaceous biomass with Clostridium sp. Q.D, and allowing sufficient time for hydrolysis and fermentation of the carbonaceous biomass by Clostridium phytofermentans to produce a fermentation end-product that is the aspartic acid, aspartate, glutamic acid, glutamate, malic acid, or malate. In one embodiment, the fermentation end-product is aspartic acid, aspartate, glutamic acid, glutamate, malic acid, or malate.

In another aspect of the invention, a system is provided for producing a fermentation end-product comprising a fermentation vessel comprising a carbonaceous biomass, and a microorganism that hydrolyses and ferments pentose and hexose saccharides from the biomass, wherein the microorganism produces a first and second fermentation end-products, wherein the first fermentation end-product is not ethanol, propanol, isopropanol, n-butanol, hydrogen, formic acid, lactic acid acetic acid, formate, lactate or acetate, wherein the fermentation vessel is adapted to provide suitable conditions for fermentation of pentose and hexose saccharides into the first and second fermentation end-products.

In another aspect of the invention, a system is provided for producing a fermentation end-product comprising a fermentation vessel comprising a carbonaceous biomass, and a Clostridium sp. Q.D, microorganism, wherein the fermentation vessel is adapted to provide suitable conditions for fermentation of pentose and hexose saccharides into the fermentation end-product.

In another aspect of the invention, a system is provided for producing a fermentation end-product comprising a fermentation vessel comprising a carbonaceous biomass and a microorganism that hydrolyses and ferments pentose and hexose saccharides from the biomass, wherein the microorganism is genetically modified or mutagenized to produce a higher concentration of the fermentation end-product compared to a non genetically modified or mutagenized form of the microorganism, wherein the fermentation vessel is adapted to provide suitable conditions for fermentation of pentose and hexose saccharides into the fermentation end-product.

In another aspect of the invention, a system is provided for producing a fermentation end-product comprising a fermentation vessel comprising a carbonaceous biomass, and a genetically modified or mutagenized microorganism that hydrolyses and ferments pentose and hexose saccharides from the biomass, wherein a non genetically modified or non mutagenized strain of the microorganism is genetically modified or mutagenized to produce the fermentation end-product, wherein the fermentation vessel is adapted to provide suitable conditions for fermentation of pentose and hexose saccharides into the fermentation end-product.

In one embodiment, the microorganism is a bacterium. In one embodiment, the microorganism is a species of Clostridia. In one embodiment, the microorganism is Clostridium phytofermentans. In one embodiment, the microorganism is Clostridium sp. Q.D. In one embodiment, the microorganism is non-recombinant. In one embodiment, the microorganism is recombinant. In one embodiment, the microorganism is genetically modified or mutagenized to enhance production of a fermentation end-product. In one embodiment, the microorganism is genetically modified to express a protein encoded by a heterologous polynucleotide. In one embodiment, the microorganism is genetically modified to enhance expression of an endogenous protein by deregulation of an endogenous promoter or by expressing an additional copy of an endogenous polynucleotide encoding the protein. In one embodiment, the non-genetically modified strain of the microorganism cannot produce the fermentation end-product. In one embodiment, the fermentation end-product is aspartic acid, aspartate, glutamic acid, glutamate, malic acid, or malate. In one embodiment, the first fermentation end-product is aspartic acid, aspartate, glutamic acid, glutamate, malic acid, or malate. In one embodiment, the fermentation end-product is not ethanol, propanol, isopropanol, n-butanol, hydrogen, formic acid, lactic acid acetic acid, formate, lactate or acetate. In one embodiment, the fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene. In one embodiment, the fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene. In one embodiment, the second fermentation end-product is not ethanol, propanol, isopropanol, n-butanol, hydrogen, formic acid, lactic acid acetic acid, formate, lactate or acetate. In one embodiment, the second fermentation end-product is ethanol, propanol, isopropanol, n-butanol, hydrogen, formic acid, lactic acid acetic acid, formate, lactate or acetate and is present at a lower amount than the first fermentation product. In one embodiment, the second fermentation end-product is ethanol, propanol, isopropanol, n-butanol, hydrogen, formic acid, lactic acid acetic acid, formate, lactate or acetate and is present at an amount less than 99% of the amount of the first fermentation product. In one embodiment, the second fermentation end-product is ethanol, propanol, isopropanol, n-butanol, hydrogen, formic acid, lactic acid acetic acid, formate, lactate or acetate and is present at an amount less than 75% of the amount of the first fermentation product. In one embodiment, the second fermentation end-product is ethanol, propanol, isopropanol, n-butanol, hydrogen, formic acid, lactic acid acetic acid, formate, lactate or acetate and is present at an amount less than 50% of the amount of the first fermentation product. In one embodiment, the second fermentation end-product is ethanol, propanol, isopropanol, n-butanol, hydrogen, formic acid, lactic acid acetic acid, formate, lactate or acetate and is present at an amount less than 30% of the amount of the first fermentation product. In one embodiment, the second fermentation end-product is ethanol, propanol, isopropanol, n-butanol, hydrogen, formic acid, lactic acid acetic acid, formate, lactate or acetate and is present at an amount less than 20% of the amount of the first fermentation product. In one embodiment, the second fermentation end-product is ethanol, propanol, isopropanol, n-butanol, hydrogen, formic acid, lactic acid acetic acid, formate, lactate or acetate and is present at an amount less than 10% of the amount of the first fermentation product. In one embodiment, the second fermentation end-product is ethanol, propanol, isopropanol, n-butanol, hydrogen, formic acid, lactic acid acetic acid, formate, lactate or acetate and is present at an amount less than 5% of the amount of the first fermentation product.

In one embodiment, the biomass comprises one or more of xylan, cellulose, hemicellulose, fructose, glucose, mannose, rhamnose, or xylose. In one embodiment, the biomass is plant matter. In one embodiment, the biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae. In one embodiment, the biomass comprises a higher concentration of oligomeric carbohydrates relative to monomeric carbohydrates. In one embodiment, the hydrolysis provides results in a greater concentration of cellobiose relative to monomeric carbohydrates. In one embodiment, the monomeric carbohydrates comprise xylose and arabinose. In one embodiment, the biomass is pre-treated with an acid, alkali or heat prior to contact with the microorganism. In one embodiment, the system further comprises a second species of microorganism. In one embodiment, the second species of microorganism is a yeast. In one embodiment, the second species of microorganism is S. cerevisiae or Aspergillus niger.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the synthesis of glycerol by C. phytofermentans.

FIG. 2 depicts the synthesis of xylitol or arabitol by C. phytofermentans.

FIG. 3 depicts the synthesis of sorbitol by C. phytofermentans.

FIG. 4 depicts the synthesis of butanediol by C. phytofermentans.

FIG. 5 depicts the synthesis of butanol by C. phytofermentans.

FIG. 6 depicts the synthesis of itaconic acid by C. phytofermentans.

FIG. 7 depicts the synthesis of glutamic acid by C. phytofermentans.

FIG. 8 depicts the synthesis of glucaric acid by C. phytofermentans.

FIG. 9 depicts the synthesis of levulinic acid by C. phytofermentans.

FIG. 10 depicts the synthesis of 2,5-Furandicarboxylic acid by C. phytofermentans.

FIG. 11 depicts the synthesis of aspartic acid by C. phytofermentans.

FIG. 12 depicts the synthesis of 1,4 diacid (succinic acid, fumaric acid, and malic acid) by C. phytofermentans.

FIG. 13 depicts the synthesis of 3-hydroxy propionic acid by C. phytofermentans.

FIG. 14 depicts the synthesis of the terpenoid backbone by C. phytofermentans.

FIG. 15 illustrates a method for producing fermentation end products from biomass by first treating biomass with an acid at elevated temperature and pressure in a hydrolysis unit.

FIG. 16 illustrates a method for producing fermentation end products from biomass by using solvent extraction or separation methods.

FIG. 17 illustrates a method for producing fermentation end products from biomass by charging biomass to a fermentation vessel.

FIG. 18 illustrates pretreatments that produce hexose or pentose saccharides or oligomers that are then unprocessed or processed further and either fermented separately or together.

FIG. 19 illustrates a plasmid map for pIMP1.

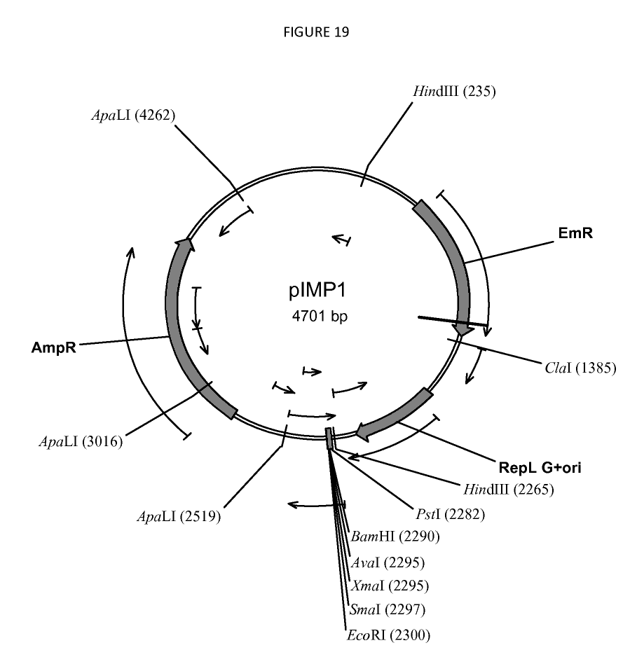

FIG. 20 illustrates a plasmid map for pIMCphy.

FIG. 21 illustrates a plasmid map for pCphyP3510.

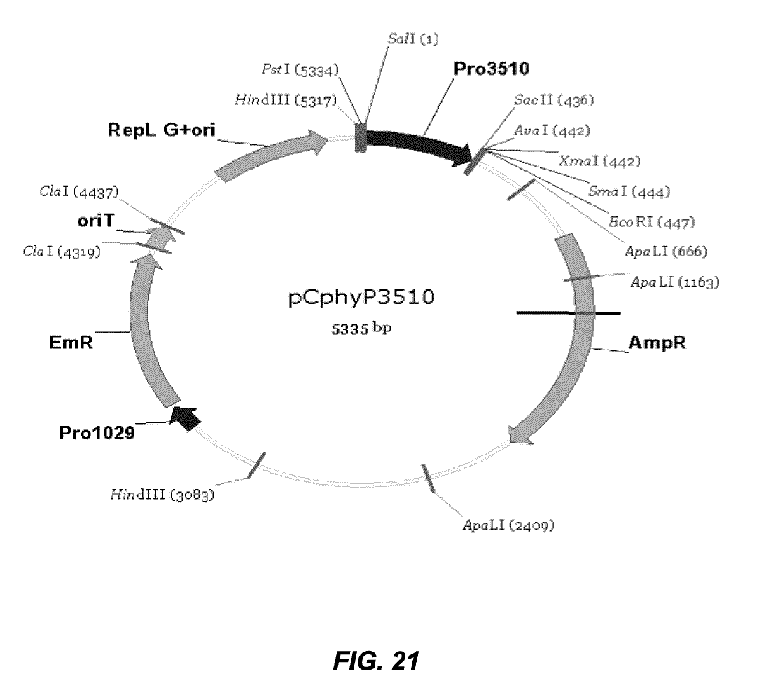

FIG. 22 illustrates a plasmid map for pCphyP3510-1163.

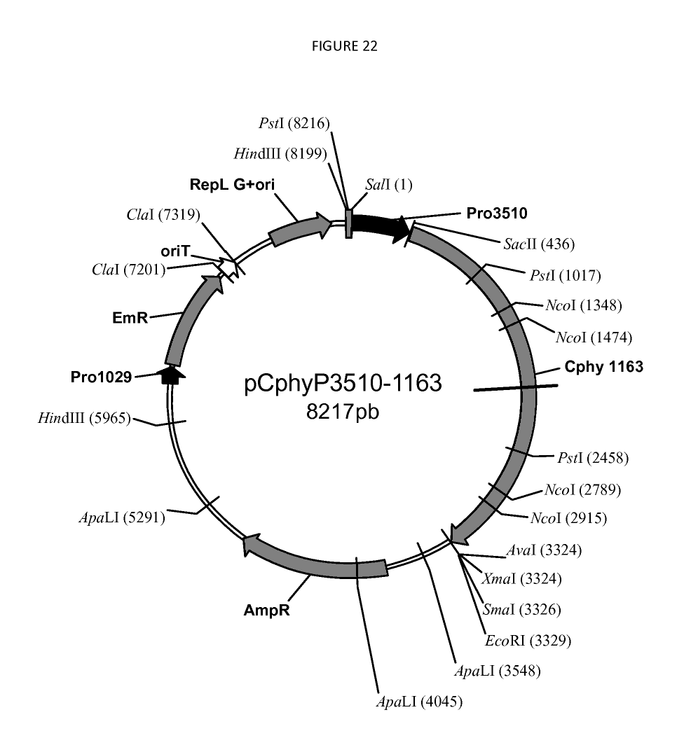

FIG. 23 illustrates mass spectrometry data identifying aspartic acid produced from a C. phy strain Q.8. Samples were extracted from cultures and then analyzed. Standard and sample chormatograms are shown, FIG. 23A and FIG. 23B, respectively.

FIG. 24 illustrates mass spectrometry data identifying malic acid produced from a C. phy strain Q.8. Samples were extracted from cultures and then analyzed. Standard and sample chormatograms are shown, FIG. 24A and FIG. 24B, respectively.

FIG. 25 illustrates mass spectrometry data identifying glutamic acid produced from a C. phy strain Q.8. Samples were extracted from cultures and then analyzed. Standard and sample chormatograms are shown, FIG. 25A and FIG. 25B, respectively.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference.

### DETAILED DESCRIPTION OF THE INVENTION

The following description and examples illustrate embodiments of the present invention in detail. It is to be understood that this invention is not limited to the particular methodology, protocols, cell lines, constructs and reagents described herein and as such can vary. Those of skill in the art will recognize that there are numerous variations and modifications of this invention that are encompassed within its scope.

Generally, the methods and compositions described herein comprise saccharification and fermentation of various biomass substrates to desired fermentation end-products.

In one embodiment, products include modified, mutant and recombinant strains of C. phytofermentans that can be used in production of chemicals from lignocellulosic, cellulosic, hemicellulosic, algal and other plant-based feedstocks or plant polysaccharides. Described herein are also methods of producing compounds, including but not limited to 1,4 diacid (succinic, fumaric and malic), 2,5 furan dicarboxylic acid, 3-hydroxy propionic acid, aspartic acid, glucaric acid, glutamic acid, itaconic acid, levulinic acid, 3-hydroxybutyrolactone, glycerol, sorbitol, xylitol/arabinitol, butanediol, butanol, and terpenoids (such as isopentenyl diphosphate) using recombinant C. phytofermentans bacteria.

In another embodiment, organisms are genetically-modified strains of C. phytofermentans comprising altered expression or structure of a gene or genes relative to the original organisms strain, wherein such genetic modifications result in increased efficiency of chemical production. In some embodiments, the genetic modifications are introduced by genetic recombination. In some embodiments, the genetic modifications are introduced by nucleic acid transformation. In further embodiments, the genetic modifications encompass inactivation of one or more genes of C. phytofermentans. In some strains, genetic modification can comprise inactivation of one or more endogenous nucleic acid sequence(s) and also comprise introduction and activation of heterologous or exogenous nucleic acid sequence(s) and promoters.

In some variations, the recombinant C. phytofermentans organisms described herein comprises a heterologous nucleic acid sequence. In some variations, the recombinant C. phytofermentans comprise one or more introduced heterologous nucleic acid(s). In some embodiments, the heterologous nucleic acid sequence is controlled by an inducible promoter. In some variations, expression of the heterologous nucleic acid sequence is controlled by a constitutive promoter.

The discovery that C. phytofermentans microbes can produce a variety of chemical products is a great advantage over other fermenting organisms. C. phytofermentans is capable of simultaneous hydrolysis and fermentation of a variety of feedstocks comprised of cellulosic, hemicellulosic or lignocellulosic materials, thus eliminating or drastically reducing the need for hydrolysis of polysaccharides prior to fermentation of sugars. In fact, C. phytofermentans preferentially takes up oligomeric polysaccharides, reducing the time and costs of hydrolysis and fermentation processes, making it an ideal organism for commercial chemical production. Further, C. phytofermentans utilizes both hexose and pentose polysaccharides and sugars, producing a highly efficient yield from feedstocks.

### DEFINITIONS

Unless characterized differently, technical and scientific terms used herein have the same meaning as commonly understood by one of ordinary skill in the art to which this invention belongs.

The term “enzyme reactive conditions” as used herein, refers to an environmental condition (i.e., such factors as temperature, pH, lack of inhibiting substances) which will permit the enzyme to function. Enzyme reactive conditions can be either in vitro, such as in a test tube, or in vivo, such as within a cell.

The term “about” as used herein, refers to a range that is 15% plus or minus from a stated numerical value within the context of the particular usage. For example, about 10 would include a range from 8.5 to 11.5.

The terms “function” and “functional” as used herein refer to biological or enzymatic function.

The term “gene” as used herein, refers to a unit of inheritance that occupies a specific locus on a chromosome and consists of transcriptional and/or translational regulatory sequences and/or a coding region and/or non-translated sequences (i.e., introns, 5′ and 3′ untranslated sequences). The term “host cell” includes an individual cell or cell culture which can be or has been a recipient of any recombinant vector(s) or isolated polynucleotide. Host cells include progeny of a single host cell, and the progeny can not necessarily be completely identical (in morphology or in total DNA complement) to the original parent cell due to natural, accidental, or deliberate mutation and/or change. A host cell includes cells transfected, transformed, or infected in vivo or in vitro with a recombinant vector or a polynucleotide. A host cell which comprises a recombinant vector is a recombinant host cell, recombinant cell, or recombinant microorganism.

The term “isolated” as used herein, refers to material that is substantially or essentially free from components that normally accompany it in its native state. For example, an “isolated polynucleotide”, as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell, i.e., it is not associated with in vivo substances.

The term “increased” or “increasing” as used herein, refers to the ability of one or more recombinant microorganisms to produce a greater amount of a given product or molecule (e.g., commodity chemical, biofuel, or intermediate product thereof) as compared to a control microorganism, such as an unmodified microorganism or a differently-modified microorganism. An “increased” amount is typically a “statistically significant” amount, and can include an increase that is 1, 2, 3, 4, 5, 6, 7, 8, 9, 10, 15, 20, 30 or more times (including all integers and decimal points in between, e.g., 1.5, 1.6, 1.7. 1.8, etc.) the amount produced by an unmodified microorganism or a differently modified microorganism.

The term “operably linked” as used herein means placing a gene under the regulatory control of a promoter, which then controls the transcription and optionally the translation of the gene. In one example for the construction of promoter/structural gene combinations, the genetic sequence or promoter is positioned at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, a regulatory sequence element can be positioned with respect to a gene to be placed under its control in the same position as the element is situated in its in its natural setting with respect to the native gene it controls.

The term “Constitutive promoter” refers to a polynucleotide sequence that induces transcription or is typically active, (i.e., promotes transcription), under most conditions, such as those that occur in a host cell. A constitutive promoter is generally active in a host cell through a variety of different environmental conditions.

The term “Inducible promoter” refers to a polynucleotide sequence that induces transcription or is typically active only under certain conditions, such as in the presence of a specific transcription factor or transcription factor complex, a given molecule factor (e.g., IPTG) or a given environmental condition (e.g., CO₂ concentration, nutrient levels, light, heat). In the absence of that condition, inducible promoters typically do not allow significant or measurable levels of transcriptional activity.

The terms “polynucleotide” or “nucleic acid” as used herein designates mRNA, RNA, cRNA, rRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

As will be understood by those skilled in the art, a polynucleotide sequence can include genomic sequences, extra-genomic and plasmid-encoded sequences and smaller engineered gene segments that express, or can be adapted to express, proteins, polypeptides, peptides and the like. Such segments can be naturally isolated, or modified synthetically by the hand of man.

Polynucleotides can be single-stranded (coding or antisense) or double-stranded, and can be DNA (genomic, cDNA or synthetic) or RNA molecules. In one embodiment additional coding or non-coding sequences can, be present within a polynucleotide. In another embodiment a polynucleotide can be linked to other molecules and/or support materials.

Polynucleotides can comprise a native sequence (i.e., an endogenous sequence) or can comprise a variant, or a biological functional equivalent of such a sequence. Polynucleotide variants can contain one or more base substitutions, additions, deletions and/or insertions, as further described below. In one embodiment a polynucleotide variant encodes a polypeptide with the same sequence as the native protein. In another embodiment a polynucleotide variant encodes a polypeptide with substantially similar enzymatic activity as the native protein. In another embodiment a polynucleotide variant encodes a protein with increased enzymatic activity relative to the native polypeptide. The effect on the enzymatic activity of the encoded polypeptide can generally be assessed as described herein.

A polynucleotide encoding a polypeptide can be combined with other DNA sequences, such as promoters, polyadenylation signals, additional restriction enzyme sites, multiple cloning sites, other coding segments, and the like, such that their overall length can vary considerably. In one embodiment the maximum length of a polynucleotide sequence which can be used to transform a microorganism is governed only by the nature of the recombinant protocol employed.

The terms “polynucleotide variant” and “variant” and the like refer to polynucleotides that display substantial sequence identity with any of the reference polynucleotide sequences or genes described herein, and to polynucleotides that hybridize with any polynucleotide reference sequence described herein, or any polynucleotide coding sequence of any gene or protein referred to herein, under low stringency, medium stringency, high stringency, or very high stringency conditions that are defined hereinafter and known in the art. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide, or has increased activity in relation to the reference polynucleotide (i.e., optimized). Polynucleotide variants include, for example, polynucleotides having at least 50% (and at least 51% to at least 99% and all integer percentages in between) sequence identity with a reference polynucleotide described herein.

The terms “polynucleotide variant” and “variant” also include naturally-occurring allelic variants that encode these enzymes. Examples of naturally-occurring variants include allelic variants (same locus), homologs (different locus), and orthologs (different microorganism). Naturally occurring variants such as these can be identified and isolated using well-known molecular biology techniques including, for example, various polymerase chain reaction (PCR) and hybridization-based techniques as known in the art. Naturally-occurring variants can be isolated from any microorganism that encodes one or more genes having a suitable enzymatic activity described herein (e.g., C—C ligase, diol dehyodrogenase, pectate lyase, alginate lyase, diol dehydratase, transporter, etc.).

Non-naturally occurring variants can be made by mutagenesis techniques, including those applied to polynucleotides, cells, or microorganisms. The variants can contain nucleotide substitutions, deletions, inversions and insertions. Variation can occur in either or both the coding and non-coding regions. In certain aspects, non-naturally occurring variants can have been optimized for use in a given microorganism (e.g., E. coli), such as by engineering and screening the enzymes for increased activity, stability, or any other desirable feature. The variations can produce both conservative and non-conservative amino acid substitutions (as compared to the originally encoded product). For polynucleotide sequences, conservative variants include those sequences that, because of the degeneracy of the genetic code, encode the amino acid sequence of a reference polypeptide. Variant polynucleotide sequences also include synthetically derived nucleotide sequences, such as those generated, for example, by using site-directed mutagenesis but which still encode a biologically active polypeptide. Generally, variants of a reference polynucleotide sequence will have at least about 30%, 40% 50%, 55%, 60%, 65%, 70%, generally at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more sequence identity with the reference polynucleotide sequence as determined by sequence alignment programs described elsewhere herein using default parameters. In one embodiment a variant polynucleotide sequence encodes a protein with substantially similar activity compared to a protein encoded by the respective reference polynucleotide sequence. Substantially similar activity means variant protein activity that is within +/−15% of the activity of a protein encoded by the respective reference polynucleotide sequence. In another embodiment a variant polynucleotide sequence encodes a protein with greater activity compared to a protein encoded by the respective reference polynucleotide sequence.

The terms “hybridizes under low stringency, hybridizes medium stringency, hybridizes high stringency, or hybridizes very high stringency conditions” as used herein, refers to conditions for hybridization and washing. Guidance for performing hybridization reactions can be found in Ausubel et al., “Current Protocols in Molecular Biology”, John Wiley & Sons Inc, 1994-1998, Sections 6.3.1-6.3.6. Aqueous and non-aqueous methods are described in that reference and either can be used.

The term “low stringency” as used herein, refers to conditions that include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also can include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6× sodium chloride/sodium citrate (SSC) at about 45° C., followed by two washes in 0.2×SSC, 0.1% SDS at least at 50° C. (the temperature of the washes can be increased to 55° C. for low stringency conditions).

The term “Medium stringency” as used herein, refers to conditions that include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also can include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C.

The term “High stringency” as used herein, refers to conditions that include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also can include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

Due to the degeneracy of the genetic code, amino acids can be substituted for other amino acids in a protein sequence without appreciable loss of the desired activity (see Table 1 below). It is thus contemplated that various changes can be made in the peptide sequences of the disclosed protein sequences, or their corresponding nucleic acid sequences without appreciable loss of the biological activity.

In making such changes, the hydropathic index of amino acids can be considered. The importance of the hydropathic amino acid index in conferring interactive biological function on a protein is generally understood in the art (Kyte and Doolittle, J. Mol. Biol., 157: 105-132, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Amino acids have been assigned a hydropathic index on the basis of their hydrophobicity and charge characteristics. These are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

It is known in the art that certain amino acids can be substituted by other amino acids having a similar hydropathic index or score and result in a protein with similar biological activity, i.e., still obtain a biologically-functional protein. In one embodiment, the substitution of amino acids whose hydropathic indices are within +/−0.2 is preferred, those within +/−0.1 are more preferred, and those within +/−.0.5 are most preferred.

It is also understood in the art that the substitution of like amino acids can be made effectively on the basis of hydrophilicity. U.S. Pat. No. 4,554,101 (Hopp, which is herein incorporated by reference in its entirety) states that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0.+-0.1); serine (+0.3); asparagine/glutamine (+0.2); glycine (0); threonine (−0.4); proline (−0.5.+-0.1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4).

It is understood that an amino acid can be substituted by another amino acid having a similar hydrophilicity score and still result in a protein with similar biological activity, i.e., still obtain a biologically functional protein. In one embodiment the substitution of amino acids whose hydropathic indices are within +/−0.2 is preferred, those within +/−0.1 are more preferred, and those within +/−.0.5 are most preferred.

As outlined above, amino acid substitutions can be based on the relative similarity of the amino acid side-chain substituents, for example, their hydrophobicity, hydrophilicity, charge, size, and the like. Exemplary substitutions which take any of the foregoing characteristics into consideration are well known to those of skill in the art and include: arginine and lysine; glutamate and aspartate; serine and threonine; glutamine and asparagine; and valine, leucine, and isoleucine. Changes which are not expected to be advantageous can also be used if these resulting proteins have the same or improved characteristics, relative to the unmodified polypeptide from which they are engineered.

In one embodiment a polynucleotide comprises codons in its protein coding sequence that are optimized to increase the thermostability of an mRNA transcribed from the polynucleotide. In one embodiment this optimization does not change the amino acid sequence encoded by the polynucleotide. In another embodiment a polynucleotide comprises codons in its protein coding sequence that are optimized to increase translation efficiency of an mRNA from the polynucleotide in a host cell. In one embodiment this optimization does not change the amino acid sequence encoded by the polynucleotide.

The RNA codon table below (Table 1) shows the 64 codons and the amino acid for each. The direction of the mRNA is 5′ to 3′.

TABLE 1

| | | 2nd base | | | |
|---|---|---|---|---|---|
| | | U | C | A | G |
| 1st | U | UUU (Phe/F) | UCU (Ser/S) | UAU (Tyr/Y) | UGU (Cys/C) |
| base | | Phenylalanine | Serine | Tyrosine | Cysteine |
| | | UUC (Phe/F) | UCC (Ser/S) | UAC (Tyr/Y) | UGC (Cys/C) |
| | | Phenylalanine | Serine | Tyrosine | Cysteine |
| | | UUA (Leu/L) | UCA (Ser/S) | UAA Ochre | UGA Opal |
| | | Leucine | Serine | (Stop) | (Stop) |
| | | UUG (Leu/L) | UCG (Ser/S) | UAG Amber | UGG (Trp/W) |
| | | Leucine | Serine | (Stop) | Tryptophan |
| | C | CUU (Leu/L) | CCU (Pro/P) | CAU (His/H) | CGU (Arg/R) |
| | | Leucine | Proline | Histidine | Arginine |
| | | CUC (Leu/L) | CCC (Pro/P) | CAC (His/H) | CGC (Arg/R) |
| | | Leucine | Proline | Histidine | Arginine |
| | | CUA (Leu/L) | CCA (Pro/P) | CAA (Gln/Q) | CGA (Arg/R) |
| | | Leucine | Proline | Glutamine | Arginine |
| | | CUG (Leu/L) | CCG (Pro/P) | CAG (Gln/Q) | CGG (Arg/R) |
| | | Leucine | Proline | Glutamine | Arginine |
| | A | AUU (Ile/I) | ACU (Thr/T) | AAU (Asn/N) | AGU (Ser/S) |
| | | Isoleucine | Threonine | Asparagine | Serine |
| | | AUC (Ile/I) | ACC (Thr/T) | AAC (Asn/N) | AGC (Ser/S) |
| | | Isoleucine | Threonine | Asparagine | Serine |
| | | AUA (Ile/I) | ACA (Thr/T) | AAA (Lys/K) | AGA (Arg/R) |
| | | Isoleucine | Threonine | Lysine | Arginine |
| | | AUG^{[A]} (Met/M) | ACG (Thr/T) | AAG (Lys/K) | AGG (Arg/R) |
| | | Methionine | Threonine | Lysine | Arginine |
| | G | GUU (Val/V) | GCU (Ala/A) | GAU (Asp/D) | GGU (Gly/G) |
| | | Valine | Alanine | Aspartic acid | Glycine |
| | | GUC (Val/V) | GCC (Ala/A) | GAC (Asp/D) | GGC (Gly/G) |
| | | Valine | Alanine | Aspartic acid | Glycine |
| | | GUA (Val/V) | GCA (Ala/A) | GAA (Glu/E) | GGA (Gly/G) |
| | | Valine | Alanine | Glutamic acid | Glycine |
| | | GUG (Val/V) | GCG (Ala/A) | GAG (Glu/E) | GGG (Gly/G) |
| | | Valine | Alanine | Glutamic acid | Glycine |

The codon AUG both codes for methionine and serves as an initiation site: the first AUG in an mRNA's coding region is where translation into protein begins.

In one embodiment a method disclosed which uses variants of full-length polypeptides having any of the enzymatic activities described herein, truncated fragments of these full-length polypeptides, variants of truncated fragments, as well as their related biologically active fragments. Typically, biologically active fragments of a polypeptide can participate in an interaction, for example, an intra-molecular or an inter-molecular interaction. An inter-molecular interaction can be a specific binding interaction or an enzymatic interaction (e.g., the interaction can be transient and a covalent bond is formed or broken). Biologically active fragments of a polypeptide/enzyme an enzymatic activity described herein include peptides comprising amino acid sequences sufficiently similar to, or derived from, the amino acid sequences of a (putative) full-length reference polypeptide sequence. Typically, biologically active fragments comprise a domain or motif with at least one enzymatic activity, and can include one or more (and in some cases all) of the various active domains. A biologically active fragment of a an enzyme can be a polypeptide fragment which is, for example, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 220, 240, 260, 280, 300, 320, 340, 360, 380, 400, 450, 500, 600 or more contiguous amino acids, including all integers in between, of a reference polypeptide sequence. In certain embodiments, a biologically active fragment comprises a conserved enzymatic sequence, domain, or motif, as described elsewhere herein and known in the art. Suitably, the biologically-active fragment has no less than about 1%, 10%, 25%, or 50% of an activity of the wild-type polypeptide from which it is derived.

The term “exogenous” as used herein, refers to a polynucleotide sequence or polypeptide that does not naturally occur in a given wild-type cell or microorganism, but is typically introduced into the cell by a molecular biological technique, i.e., engineering to produce a recombinant microorganism. Examples of “exogenous” polynucleotides include vectors, plasmids, and/or man-made nucleic acid constructs encoding a desired protein or enzyme.

The term “endogenous” as used herein, refers to naturally-occurring polynucleotide sequences or polypeptides that can be found in a given wild-type cell or microorganism. For example, certain naturally-occurring bacterial or yeast species do not typically contain a benzaldehyde lyase gene, and, therefore, do not comprise an “endogenous” polynucleotide sequence that encodes a benzaldehyde lyase. In this regard, it is also noted that even though a microorganism can comprise an endogenous copy of a given polynucleotide sequence or gene, the introduction of a plasmid or vector encoding that sequence, such as to over-express or otherwise regulate the expression of the encoded protein, represents an “exogenous” copy of that gene or polynucleotide sequence. Any of the pathways, genes, or enzymes described herein can utilize or rely on an “endogenous” sequence, or can be provided as one or more “exogenous” polynucleotide sequences, and/or can be used according to the endogenous sequences already contained within a given microorganism.

The term “sequence identity” for example, comprising a “sequence 50% identical to,” as used herein, refers to the extent that sequences are identical on a nucleotide-by-nucleotide basis or an amino acid-by-amino acid basis over a window of comparison. Thus, a “percentage of sequence identity” can be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, H is, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

The terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides can each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window can comprise additions or deletions (i.e., gaps) of about 20% or less as compared to the reference sequence (which does not comprise additions or deletions) for optimal alignment of the two sequences. Optimal alignment of sequences for aligning a comparison window can be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also can be made to the BLAST family of programs as for example disclosed by Altschul et al., 1997, Nucl. Acids Res. 25:3389, which is herein incorporated by reference in its entirety. A detailed discussion of sequence analysis can be found in Unit 19.3 of Ausubel et al., “Current Protocols in Molecular Biology”, John Wiley & Sons Inc, 1994-1998, Chapter 15, which is herein incorporated by reference in its entirety.

The term “transformation” as used herein, refers to the permanent, heritable alteration in a cell resulting from the uptake and incorporation of foreign DNA into the host-cell genome. This includes the transfer of an exogenous gene from one microorganism into the genome of another microorganism as well as the addition of additional copies of an endogenous gene into a microorganism.

The term “vector” as used herein, refers to a polynucleotide molecule, such as a DNA molecule. It can be derived, from a plasmid, bacteriophage, yeast or virus, into which a polynucleotide can be inserted or cloned. A vector can contain one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. Such a vector can comprise specific sequences that allow recombination into a particular, desired site of the host chromosome. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. A vector can be one which is operably functional in a bacterial cell, such as a cyanobacterial cell. The vector can include a reporter gene, such as a green fluorescent protein (GFP), which can be either fused in frame to one or more of the encoded polypeptides, or expressed separately. The vector can also include a selection marker such as an antibiotic resistance gene that can be used for selection of suitable transformants.

The terms “wild-type” and “naturally-occurring” as used herein are used interchangeably to refer to a gene or gene product that has the characteristics of that gene or gene product when isolated from a naturally occurring source. A wild type gene or gene product (e.g., a polypeptide) is that which is most frequently observed in a population and is thus arbitrarily designed the “normal” or “wild-type” form of the gene.

Biomass

“Biomass” can include, but is not limited to, plant matter, such as woody or non-woody plant matter, crop plants, aquatic or marine biomass, fruit-based biomass such as fruit waste, and vegetable-based biomass such as vegetable waste, and animal based biomass among others. Examples of aquatic or marine biomass include, but are not limited to, kelp, other seaweed, algae, and marine microflora, microalgae, sea grass, salt marsh grasses such as Spartina sp. or Phragmites sp. and the like. The term “crop plant” is intended to encompass any plant that is cultivated or harvested for the purpose of producing plant material that is sought after by man for either oral consumption, or for utilization in an industrial, pharmaceutical, or commercial process. The invention may be applied to any of a variety of plants, including, but not limited to maize, wheat, rice, barley, soybean, cotton, sorghum, high biomass sorghum, oats, tobacco, Miscanthus grass, switch grass, trees (softwoods and hardwoods), beans in general, rape/canola, alfalfa, flax, sunflower, safflower, millet, rye, sugarcane, sugar beet, cocoa, tea, Brassica sp., cotton, coffee, sweet potato, flax, peanut, clover; vegetables such as lettuce, tomato, cucurbits, cassava, potato, carrot, radish, pea, lentils, cabbage, cauliflower, broccoli, brussels sprouts, peppers, and pineapple; tree fruits such as citrus, apples, pears, peaches, apricots, walnuts, avocado, banana, and coconut; and flowers such as orchids, carnations and roses, and nonvascular plants such as ferns, and gymnosperms such as palms. Biomass can also include genetically-modified organisms, such as recombinant algae or plants that can produce hydrolytic enzymes (such as cellulases, hemicellulases, or pectinases etc.) at or near the end of their life cycles. Such biomass can encompass mutated species as well as those that initiate the breakdown of cell wall components.

The term “carbonaceous biomass” as used herein has its ordinary meaning as known to those skilled in the art and may include one or more biological material that can be converted into a biofuel, chemical or other product. Carbonaceous biomass can comprise municipal waste, wood, plant material, plant matter, plant extract, distillers' grains, a natural or synthetic polymer, or a combination thereof.

Plant matter can include, but is not limited to, woody plant matter, non-woody plant matter, cellulosic material, lignocellulosic material, hemicellulosic material, carbohydrates, pectin, starch, inulin, fructans, glucans, corn, corn stover, sugar cane, grasses, stillage, leaves, switch grass, bamboo, sorghum, high biomass sorghum, and material derived from these. Plant matter can be derived from a genetically modified plant. Plant matter can be further described by reference to the chemical species present, such as proteins, polysaccharides and oils.

In one embodiment, biomass does not include fossilized sources of carbon, such as hydrocarbons that are typically found within the top layer of the Earth's crust (e.g., natural gas, nonvolatile materials composed of almost pure carbon, like anthracite coal, etc.).

Examples of fruit and/or vegetable biomass include, but are not limited to, any source of pectin such as plant peel and pomace including citrus, orange, grapefruit, potato, tomato, grape, mango, gooseberry, carrot, sugar-beet, and apple, among others. In one embodiment plant matter is characterized by the chemical species present, such as proteins, polysaccharides and oils. In one embodiment plant matter includes agricultural waste byproducts or side streams such as pomace, corn steep liquor, corn steep solids, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, pits, fermentation waste, straw, lumber, sewage, garbage or food leftovers. These materials can come from farms, forestry, industrial sources, households, etc. In another embodiment biomass comprises animal matter, including, for example milk, meat, fat, animal processing waste, and animal waste. The term “feedstock” is frequently used to refer to biomass being used for a process, such as those described herein.

The term “broth” as used herein has its ordinary meaning as known to those skilled in the art and can include the entire contents of the combination of soluble and insoluble matter, suspended matter, cells and medium, such as for example the entire contents of a fermentation reaction can be referred to as a fermentation broth.

The term “productivity” as used herein has its ordinary meaning as known to those skilled in the art and can include the mass of a material of interest produced in a given time in a given volume. Units can be, for example, grams per liter-hour, or some other combination of mass, volume, and time. In fermentation, productivity is frequently used to characterize how fast a product can be made within a given fermentation volume. The volume can be referenced to the total volume of the fermentation vessel, the working volume of the fermentation vessel, or the actual volume of broth being fermented. The context of the phrase will indicate the meaning intended to one of skill in the art. Productivity (e.g. g/L/d) is different from “titer” (e.g. g/L) in that productivity includes a time term, and titer is analogous to concentration.

The term “saccharification” as used herein has its ordinary meaning as known to those skilled in the art and can include conversion of plant polysaccharides to lower molecular weight species that can be used by the microorganism at hand. For some microorganisms, this would include conversion to monosaccharides, disaccharides, trisaccharides, and oligosaccharides of up to about seven monomer units, as well as similar sized chains of sugar derivatives and combinations of sugars and sugar derivatives. For some microorganisms, the allowable chain-length can be longer (e.g. 8, 9, 10, 11, 12, 13, 14, 15, 16, 17, 18, 19, or 20 monomer units or more) and for some microorganisms the allowable chain-length can be shorter (e.g. 1, 2, 3, 4, 5, 6, or 7 monomer units).

The term “external source” as it relates to a quantity of an enzyme or enzymes provided to a product or a process means that the quantity of the enzyme or enzymes is not produced by a microorganism in the product or process. An external source of an enzyme can include, but is not limited to an enzyme provided in purified form, cell extracts, culture medium or an enzyme obtained from a commercially available source.

The term “biocatalyst” as used herein has its ordinary meaning as known to those skilled in the art and can include one or more enzymes and/or microorganisms, including solutions, suspensions, and mixtures of enzymes and microorganisms. In some contexts this word will refer to the possible use of either enzymes or microorganisms to serve a particular function, in other contexts the word will refer to the combined use of the two, and in other contexts the word will refer to only one of the two. The context of the phrase will indicate the meaning intended to one of skill in the art.

The terms “conversion efficiency” or “yield” as used herein have their ordinary meaning as known to those skilled in the art and can include the mass of product made from a mass of substrate. The term can be expressed as a percentage yield of the product from a starting mass of substrate. For the production of ethanol from glucose, the net reaction is generally accepted as:

C₆H₁₂O₆→2C₂H₅OH+2CO₂

and the theoretical maximum conversion efficiency or yield is 51% (wt.). Frequently, the conversion efficiency will be referenced to the theoretical maximum, for example, “80% of the theoretical maximum.” In the case of conversion of glucose to ethanol, this statement would indicate a conversion efficiency of 41% (wt.). The context of the phrase will indicate the substrate and product intended to one of skill in the art. For substrates comprising a mixture of different carbon sources such as found in biomass (xylan, xylose, glucose, cellobiose, arabinose cellulose, hemicellulose etc.), the theoretical maximum conversion efficiency of the biomass to ethanol is an average of the maximum conversion efficiencies of the individual carbon source constituents weighted by the relative concentration of each carbon source. In some cases, the theoretical maximum conversion efficiency is calculated based on an assumed saccharification yield. In one embodiment, given carbon source comprising 10 g of cellulose, the theoretical maximum conversion efficiency can be calculated by assuming saccharification of the cellulose to the assimilable carbon source glucose of about 75% by weight. In this embodiment, 10 g of cellulose can provide 7.5 g of glucose which can provide a maximum theoretical conversion efficiency of about 7.501% or 3.8 g of ethanol. In other cases, the efficiency of the saccharification step can be calculated or determined, i.e., saccharification yield. Saccharification yields can include between about 10-100%, about 20-90%, about 30-80%, about 40-70% or about 50-60%, such as about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% for any carbohydrate carbon sources larger than a single monosaccharide subunit.

The saccharification yield takes into account the amount of ethanol, and acidic products produced plus the amount of residual monomeric sugars detected in the media. The ethanol figures resulting from media components are not adjusted in this experiment. These can account for up to 3 g/l ethanol production or equivalent of up to 6 g/l sugar as much as +/−10%-15% saccharification yield (or saccharification efficiency). For this reason the saccharification yield % can be greater than 100% for some plots. The terms “fed-batch” or “fed-batch fermentation” as used herein has its ordinary meaning as known to those skilled in the art and can include a method of culturing microorganisms where nutrients, other medium components, or biocatalysts (including, for example, enzymes, fresh microorganisms, extracellular broth, etc.) are supplied to the fermentor during cultivation, but culture broth is not harvested from the fermentor until the end of the fermentation, although it can also include “self seeding” or “partial harvest” techniques where a portion of the fermentor volume is harvested and then fresh medium is added to the remaining broth in the fermentor, with at least a portion of the inoculum being the broth that was left in the fermentor. In some embodiments, a fed-batch process might be referred to with a phrase such as, “fed-batch with cell augmentation.” This phrase can include an operation where nutrients and microbial cells are added or one where microbial cells with no substantial amount of nutrients are added. The more general phrase “fed-batch” encompasses these operations as well. The context where any of these phrases is used will indicate to one of skill in the art the techniques being considered.

A term “phytate” as used herein has its ordinary meaning as known to those skilled in the art can be include phytic acid, its salts, and its combined forms as well as combinations of these.

The term “fermentable sugars” as used herein has its ordinary meaning as known to those skilled in the art and may include one or more sugars and/or sugar derivatives that can be utilized as a carbon source by the microorganism, including monomers, dimers, and polymers of these compounds including two or more of these compounds. In some cases, the microorganism may break down these polymers, such as by hydrolysis, prior to incorporating the broken down material. Exemplary fermentable sugars include, but are not limited to glucose, xylose, arabinose, galactose, mannose, rhamnose, cellobiose, lactose, sucrose, maltose, and fructose.

The term “plant polysaccharide” as used herein has its ordinary meaning as known to those skilled in the art and may comprise one or more carbohydrate polymers of sugars and sugar derivatives as well as derivatives of sugar polymers and/or other polymeric materials that occur in plant matter. Exemplary plant polysaccharides include lignin, cellulose, starch, pectin, and hemicellulose. Others are chitin, sulfonated polysaccharides such as alginic acid, agarose, carrageenan, porphyran, furcelleran and funoran. Generally, the polysaccharide can have two or more sugar units or derivatives of sugar units. The sugar units and/or derivatives of sugar units may repeat in a regular pattern, or otherwise. The sugar units can be hexose units or pentose units, or combinations of these. The derivatives of sugar units can be sugar alcohols, sugar acids, amino sugars, etc. The polysaccharides can be linear, branched, cross-linked, or a mixture thereof. One type or class of polysaccharide can be cross-linked to another type or class of polysaccharide. Plant polysaccharide can be derived from genetically modified plants.

Examples of polysaccharides, oligosaccharides, monosaccharides or other sugar components of biomass include, but are not limited to, alginate, agar, carrageenan, fucoidan, pectin, gluronate, mannuronate, mannitol, lyxose, cellulose, hemicellulose, glycerol, xylitol, glucose, mannose, galactose, xylose, xylan, mannan, arabinan, arabinose, glucuronate, galacturonate (including di- and tri-galacturonates), rhamnose, and the like.

Microorganisms

Microorganisms useful in compositions and methods of the invention include, but are not limited to bacteria, or yeast. Examples of bacteria include, but are not limited to, any bacterium found in the genus of Clostridium, such as C. acetobutylicum, C. aerotolerans, C. beijerinckii, C. bifermentans, C. botulinum, C. butyricum, C. cadaveris, C. chauvoei, C. clostridioforme, C. colicanis, C. difficile, C. fallax, C. formicaceticum, C. histolyticum, C. innocuum, C. ljungdahlii, C. laramie, C. lavalense, C. novyi, C. oedematiens, C. paraputrificum, C. perfringens, C. phytofermentans, C. piliforme, C. ramosum, C. scatologenes, C. septicum, C. sordellii, C. sporogenes, C. sp. Q.D, C. tertium, C. tetani, C. tyrobutyricum, and mutagenized variants thereof (e.g. C. phytofermentans Q.12 or C. phytofermentans Q.13).

Examples of yeast that can be utilized in co-culture methods of the invention include but are not limited to, species found in Cryptococcaceae, Sporobolomycetaceae with the genera Cryptococcus, Torulopsis, Pityrosporum, Brettanomyces, Candida, Kloeckera, Trigonopsis, Trichosporon, Rhodotorula and Sporobolomyces and Bullera, the families Endo- and Saccharomycetaceae, with the genera Saccharomyces, Debaromyces, Lipomyces, Hansenula, Endomycopsis, Pichia, Hanseniaspora, Saccharomyces cerevisiae, Pichia pastoris, Hansenula polymorphs, Schizosaccharomyces pombe, Kluyveromyces lactis, Zygosaccharomyces rouxii, Yarrowia lipolitica, Emericella nidulans, Aspergillus nidulans, Deparymyces hansenii and Torulaspora hansenii.

In another embodiment a microorganism can be wild type, or a genetically modified strain. In one embodiment a microorganism can be genetically modified to express one or more polypeptides capable of neutralizing a toxic by-product or inhibitor, which can result in enhanced end-product production in yield and/or rate of production. Examples of modifications include chemical or physical mutagenesis, directed evolution, or genetic alteration to enhance enzyme activity of endogenous proteins, introducing one or more heterogeneous nucleic acid molecules into a host microorganism to express a polypeptide not otherwise expressed in the host, modifying physical and chemical conditions to enhance enzyme function (e.g., modifying and/or maintaining a certain temperature, pH, nutrient concentration, or biomass concentration), or a combination of one or more such modifications.

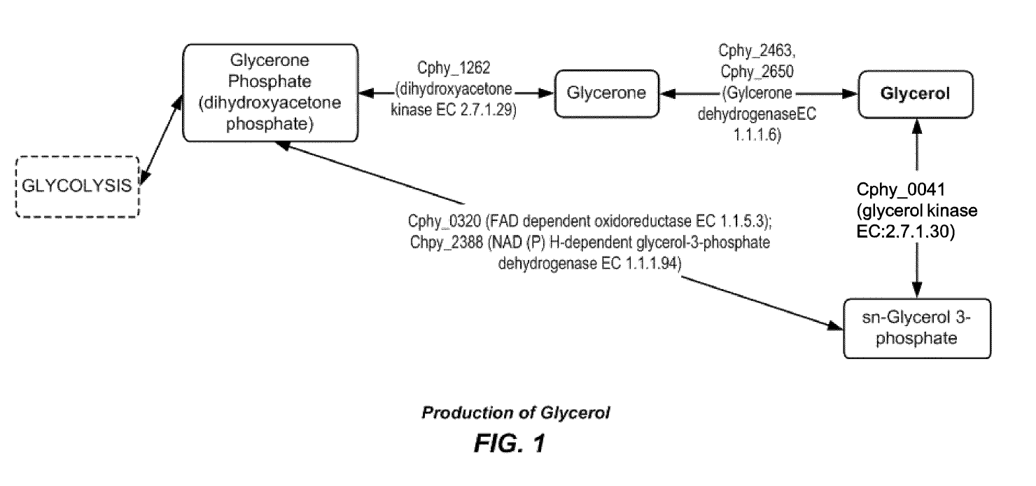

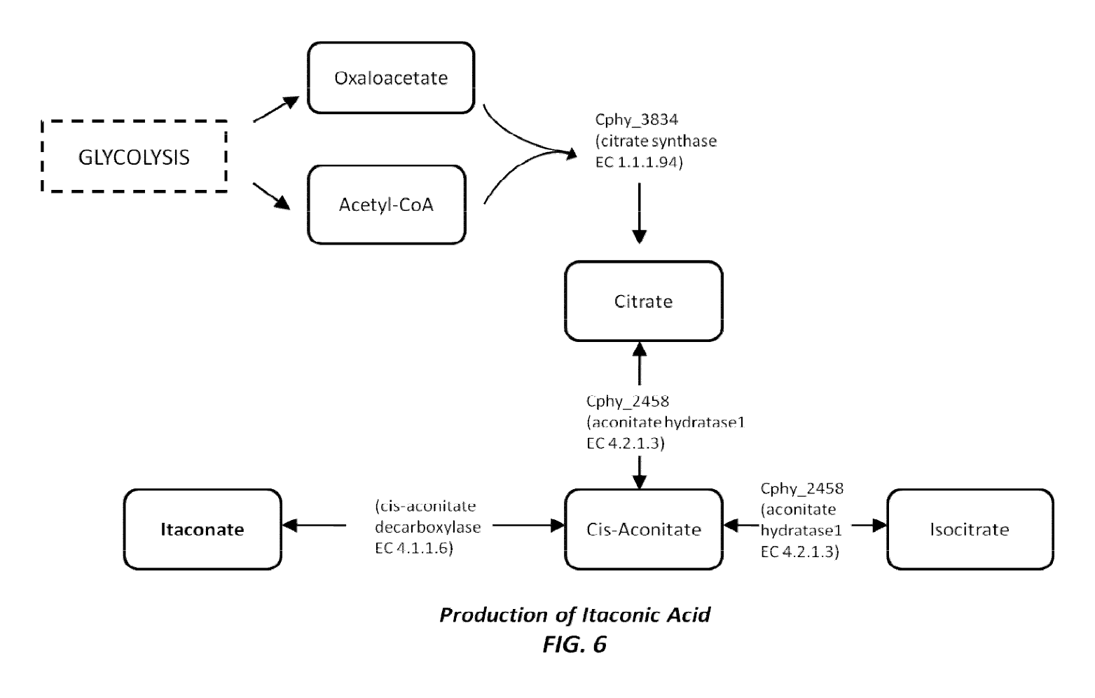

In one embodiment, a microorganism can be utilized during saccharification and/or fermentation processes to produce an end-product. In one embodiment a bacterium (e.g. Clostridia) and one or more enzymes can be cultured with a carbonaceous biomass to produce a fermentation end product which is a chemical. Examples of chemical end products, include but are not limited to 1,4 diacid (succinic, fumaric and malic), 2,5 furan dicarboxylic acid, 3-hydroxy propionic acid, aspartic acid, glucaric acid, glutamic acid, itaconic acid, levulinic acid, 3-hydroxybutyrolactone, glycerol, sorbitol, xylitol/arabinitol, butanediol, butanol, and terpenoids (such as isopentenyl diphosphate). Enzymes may be hydrolytic enzymes (e.g. cellulases, hemicellulases, or pectinases etc.) or enzymes used to process biomass to produce chemical products. Examples of such enzymes, include but are not limited to L-butanediol dehydrogenase, acetoin reductase, 3-hydroxyacyl-CoA dehydrogenase, cis-aconitate decarboxylase, dihydrorxyacetone kinase, GldA (glycerol dehydrogenase), iron-containing alcohol dehydrogenase, NAD(P)H-dependent glycerol-3-phosphate dehydrogenase, glycerol kinase, dihydroxyacetone phoshphate (DHAP), NAD⁺-dependent glycerol 3-phosphate dehydrogenase (Gpd p), glycerol 3-phosphatase (Gpp p), D-xylulose reductase, D-arabitol 4-dehydrogenase, NAD-dependent xylitol dehydrogenase, D-sorbitol dehydrogenases (GutB), D-sorbitol-6-phosphate dehydrogenase-encoding gene (gutF), L-lactate dehydrogenase, acetoin reductase/2,3-butanediol dehydrogenases (AR/BDH), D-glucitol dehydrogenase, scetolactate decarboxylase, 2,3 butanediol (2,3-BDL) acetyl-CoA acetyl transferase, 3-hydroxybutyryl-CoA dehydrogenase, crotonase, butyryl-CoA dehydrogenase, butyraldehyde dehydrogenase, butanol dehydrogenase, 3-oxoacyl-(acyl-carrier-protein) synthase 2, iron-containing alcohol dehydrogenase, cis-aconitate decarboxylase, citrate synthase, aconitase, cis-aconitic acid decarboxylase (itaconate-forming), cis-aconitic acid decarboxylase (citraconate-forming), citraconate isomerase, mitochondrial dicarboxylate-tricarboxylate antiporter, mitochondrial tricarboxylate transporter; dicarboxylate transporter; or 2-methylcitrate dehydratase, aspartase (L-aspartate ammonialyase), pyruvate carboxylase, 3-hydroxyisobutyrate dehydrogenase or the like. In one embodiment, a microorganisms may be utilized during saccharification and/or fermentation processes to produce any one of the end-products described in FIGS. 1 through 14.

In some embodiments, two or more different microorganisms can be utilized during saccharification and/or fermentation processes to produce an end-product. In one embodiment a bacterium (e.g. Clostridia) and a yeast and one or more enzymes can be cultured with a carbonaceous biomass to produce a fermentation end product which is a chemical. Examples of chemical end products, include but are not limited to 1,4 diacid (succinic, fumaric and malic), 2,5 furan dicarboxylic acid, 3-hydroxy propionic acid, aspartic acid, glucaric acid, glutamic acid, itaconic acid, levulinic acid, 3-hydroxybutyrolactone, glycerol, sorbitol, xylitol/arabinitol, butanediol, butanol, and terpenoids (such as isopentenyl diphosphate). Enzymes may be hydrolytic enzymes (e.g. cellulases, hemicellulases, or pectinases etc.) or enzymes used to process biomass to produce chemical products. Examples of such enzymes, include but are not limited to L-butanediol dehydrogenase, acetoin reductase, 3-hydroxyacyl-CoA dehydrogenase, cis-aconitate decarboxylase, dihydrorxyacetone kinase, GldA (glycerol dehydrogenase), iron-containing alcohol dehydrogenase, NAD(P)H-dependent glycerol-3-phosphate dehydrogenase, glycerol kinase, dihydroxyacetone phoshphate (DHAP), NAD⁺-dependent glycerol 3-phosphate dehydrogenase (Gpd p), glycerol 3-phosphatase (Gpp p), D-xylulose reductase, D-arabitol 4-dehydrogenase, NAD-dependent xylitol dehydrogenase, D-sorbitol dehydrogenases (GutB), D-sorbitol-6-phosphate dehydrogenase-encoding gene (gutF), L-lactate dehydrogenase, acetoin reductase/2,3-butanediol dehydrogenases (AR/BDH), D-glucitol dehydrogenase), scetolactate decarboxylase, 2,3 butanediol (2,3-BDL) acetyl-CoA acetyl transferase, 3-hydroxybutyryl-CoA dehydrogenase, crotonase, butyryl-CoA dehydrogenase, butyraldehyde dehydrogenase, butanol dehydrogenase, 3-oxoacyl-(acyl-carrier-protein) synthase 2, iron-containing alcohol dehydrogenase, cis-aconitate decarboxylase, citrate synthase, aconitase, cis-aconitic acid decarboxylase (itaconate-forming), cis-aconitic acid decarboxylase (citraconate-forming), citraconate isomerase, mitochondrial dicarboxylate-tricarboxylate antiporter, mitochondrial tricarboxylate transporter; dicarboxylate transporter; or 2-methylcitrate dehydratase, aspartase (L-aspartate ammonialyase), pyruvate carboxylase, 3-hydroxyisobutyrate dehydrogenase or the like. In one embodiment, two or more different microorganisms may be utilized during saccharification and/or fermentation processes to produce any one of the end-products described in FIGS. 1 through 14.

In one embodiment, microorganisms utilized in compositions or methods of the invention include Clostridia. One such Clostridia is C. phytofermentans, C. sp. Q.D or mutants thereof, such as C. phytofermentans Q.8, C. phytofermentans Q.12, or C. phytofermentans Q.13.

In one embodiment, microorganisms of the invention can be modified to comprise one or more heterologous polynucleotides that encode an expansin or swollenin, catalase, cellulase, hemicellulase, xylanse, or catalase enzymes. Microorganisms of the invention can be modified to comprise one or more enzymes useful for the production of chemical products, such enzymes include but are not limited to L-butanediol dehydrogenase, acetoin reductase, 3-hydroxyacyl-CoA dehydrogenase, cis-aconitate decarboxylase, dihydrorxyacetone kinase, GldA (glycerol dehydrogenase), iron-containing alcohol dehydrogenase, NAD(P)H-dependent glycerol-3-phosphate dehydrogenase, glycerol kinase, dihydroxyacetone phoshphate (DHAP), NAD⁺-dependent glycerol 3-phosphate dehydrogenase (Gpd p), glycerol 3-phosphatase (Gpp p), D-xylulose reductase, D-arabitol 4-dehydrogenase, NAD-dependent xylitol dehydrogenase, D-sorbitol dehydrogenases (GutB), D-sorbitol-6-phosphate dehydrogenase-encoding gene (gutF), L-lactate dehydrogenase, acetoin reductase/2,3-butanediol dehydrogenases (AR/BDH), D-glucitol dehydrogenase), scetolactate decarboxylase, 2,3 butanediol (2,3-BDL) acetyl-CoA acetyl transferase, 3-hydroxybutyryl-CoA dehydrogenase, crotonase, butyryl-CoA dehydrogenase, butyraldehyde dehydrogenase, butanol dehydrogenase, 3-oxoacyl-(acyl-carrier-protein) synthase 2, iron-containing alcohol dehydrogenase, cis-aconitate decarboxylase, citrate synthase, aconitase, cis-aconitic acid decarboxylase (itaconate-forming), cis-aconitic acid decarboxylase (citraconate-forming), citraconate isomerase, mitochondrial dicarboxylate-tricarboxylate antiporter, mitochondrial tricarboxylate transporter; dicarboxylate transporter; or 2-methylcitrate dehydratase, aspartase (L-aspartate ammonialyase), pyruvate carboxylase, 3-hydroxyisobutyrate dehydrogenase or the like.

A microorganism that utilized in products and processes of the invention can be capable of uptake of one or more complex carbohydrates from biomass (e.g., biomass comprises a higher concentration of oligomeric carbohydrates relative to monomeric carbohydrates).

The isolated strains disclosed herein have been deposited in the Agricultural Research Service culture Collection (NRRL), an International Depositary Authority, National Center for Agricultural Utilization Research, Agricultural Research Service, U.S. Department of Agriculture, 1815 North University Street, Peoria, Ill. 61604 U.S.A. in accordance with and under the provisions of the Budapest Treaty for the Deposit of Microorganisms, i.e., they will be stored with all the care necessary to keep them viable and uncontaminated for a period of at least five years after the most recent request for the furnishing of a sample of the deposits, and in any case, for a period of at least 30 (thirty) years after the date of deposit or for the enforceable life of any patent which may issue disclosing the cultures plus five years after the last request for a sample from the deposit. The strains were tested by the NRRL and determined to be viable. The NRRL has assigned the following NRRL deposit accession numbers to strains: Clostridium sp. Q.D (NRRL B-50361), Clostridium sp. Q.D-5 (NRRL B-50362), Clostridium sp. Q.D-7 (NRRL B-50363), Clostridium phytofermentans Q.7D (NRRL B-50364), all of which were deposited on Apr. 9, 2010. The NRRL has assigned the following NRRL deposit accession numbers to strains: Clostridium phytofermentans Q.8 (NRRL B-50351), deposited on Mar. 9, 2010; Clostridium phytofermentans Q.12 (NRRL B-50436), and Clostridium phytofermentans Q.13 (NRRL B-50437), deposited on Nov. 3, 2010. The depositor acknowledges the duty to replace the deposits should the depository be unable to furnish a sample when requested, due to the condition of the deposits. All restrictions on the availability to the public of the subject culture deposits will be irrevocably removed upon the granting of a patent disclosing them. The deposits are available as required by foreign patent laws in countries wherein counterparts of the subject application, or its progeny, are filed. However, it should be understood that the availability of a deposit does not constitute a license to practice the subject matter disclosed herein in derogation of patent rights granted by governmental action.

Pretreatment of Biomass

Described herein are also methods and compositions for pre-treating biomass prior to extraction of industrially useful end-products.

In some embodiments aerobic/anaerobic cycling is employed for the bioconversion of cellulosic/lignocellulosic material to fuels and chemicals. In some embodiments, the anaerobic microorganism can ferment biomass directly without the need of a pretreatment. In certain embodiments, feedstocks are contacted with biocatalysts capable of breaking down plant-derived polymeric material into lower molecular weight products that can subsequently be transformed by biocatalysts to fuels and/or other desirable chemicals. In some embodiments pretreatment methods can include treatment under conditions of high or low pH. High or low pH treatment includes, but is not limited to, treatment using concentrated acids or concentrated alkali, or treatment using dilute acids or dilute alkali. Alkaline compositions useful for treatment of biomass in the methods of the present invention include, but are not limited to, caustic, such as caustic lime, caustic soda, caustic potash, sodium, potassium, or calcium hydroxide, or calcium oxide. In some embodiments suitable amounts of alkaline useful for the treatment of biomass ranges from 0.01 g to 3 g of alkaline (e.g. caustic) for every gram of biomass to be treated. In some embodiments suitable amounts of alkaline useful for the treatment of biomass include, but are not limited to, about 0.01 g of alkaline (e.g. caustic), 0.02 g, 0.03 g, 0.04 g, 0.05 g, 0.075 g, 0.1 g, 0.2 g, 0.3 g, 0.4 g, 0.5 g, 0.75 g, 1 g, 2 g, or about 3 g of alkaline (e.g. caustic) for every gram of biomass to be treated.

In another embodiment, pretreatment of biomass comprises dilute acid hydrolysis. Examples of dilute acid hydrolysis treatment are disclosed in T. A. Lloyd and C. E Wyman, Bioresource Technology, (2005) 96, 1967), incorporated by reference herein in its entirety. In other embodiments, pretreatment of biomass comprises pH controlled liquid hot water treatment. Examples of pH controlled liquid hot water treatments are disclosed in N. Mosier et al., Bioresource Technology, (2005) 96, 1986, incorporated by reference herein in its entirety. In other embodiments, pretreatment of biomass comprises aqueous ammonia recycle process (ARP). Examples of aqueous ammonia recycle process are described in T. H. Kim and Y. Y. Lee, Bioresource Technology, (2005) 96, 2007, incorporated by reference herein in its entirety.

In another embodiment, the above-mentioned methods have two steps: a pretreatment step that leads to a wash stream, and an enzymatic hydrolysis step of pretreated-biomass that produces a hydrolyzate stream. In the above methods, the pH at which the pretreatment step is carried out increases progressively from dilute acid hydrolysis to hot water pretreatment to alkaline reagent based methods (AFEX, ARP, and lime pretreatments). Dilute acid and hot water treatment methods solubilize mostly hemicellulose, whereas methods employing alkaline reagents remove most lignin during the pretreatment step. As a result, the wash stream from the pretreatment step in the former methods contains mostly hemicellulose-based sugars, whereas this stream has mostly lignin for the high-pH methods. The subsequent enzymatic hydrolysis of the residual feedstock leads to mixed sugars (C5 and C6) in the alkali-based pretreatment methods, while glucose is the major product in the hydrolysate from the low and neutral pH methods. The enzymatic digestibility of the residual biomass is somewhat better for the high-pH methods due to the removal of lignin that can interfere with the accessibility of cellulase enzyme to cellulose. In some embodiments, pretreatment results in removal of about 20%, 30%, 40%, 50%, 60%, 70% or more of the lignin component of the feedstock. In other embodiments, more than 40%, 50%, 60%, 70%, 80% or more of the hemicellulose component of the feedstock remains after pretreatment. In some embodiments, the microorganism (e.g., C. phytofermentans) is capable of fermenting both five-carbon and six-carbon sugars, which can be present in the feedstock, or can result from the enzymatic degradation of components of the feedstock.

In another embodiment, a two-step pretreatment is used to remove C5 polysaccharides and other components. After washing, the second step consists of an alkali treatment to remove lignin components. The pretreated biomass is then washed prior to saccharification and fermentation. One such pretreatment consists of a dilute acid treatment at room temperature or an elevated temperature, followed by a washing or neutralization step, and then an alkaline contact to remove lignin. For example, one such pretreatment can consist of a mild acid treatment with an acid that is organic (such as acetic acid, citric acid, or oxalic acid) or inorganic (such as nitric, hydrochloric, or sulfuric acid), followed by washing and an alkaline treatment in 0.5 to 2.0% NaOH. This type of pretreatment results in a higher percentage of oligomeric to monomeric saccharides, is preferentially fermented by an organism such as C. phytofermentans or C. sp. Q.D.

In another embodiment, pretreatment of biomass comprises ionic liquid pretreatment. Biomass can be pretreated by incubation with an ionic liquid, followed by extraction with a wash solvent such as alcohol or water. The treated biomass can then be separated from the ionic liquid/wash-solvent solution by centrifugation or filtration, and sent to the saccharification reactor or vessel. Examples of ionic liquid pretreatment are disclosed in US publication No. 2008/0227162, incorporated herein by reference in its entirety.

Examples of pretreatment methods are disclosed in U.S. Pat. No. 4,600,590 to Dale, U.S. Pat. No. 4,644,060 to Chou, U.S. Pat. No. 5,037,663 to Dale. U.S. Pat. No. 5,171,592 to Holtzapple, et al., et al., U.S. Pat. No. 5,939,544 to Karstens, et al., U.S. Pat. No. 5,473,061 to Bredereck, et al., U.S. Pat. No. 6,416,621 to Karstens, U.S. Pat. No. 6,106,888 to Dale, et al., U.S. Pat. No. 6,176,176 to Dale, et al., PCT publication WO2008/020901 to Dale, et al., Felix, A., et al., Anim. Prod. 51, 47-61 (1990), Wais, A. C., Jr., et al., Journal of Animal Science, 35, No. 1, 109-112 (1972), which are incorporated herein by reference in their entireties.

In some embodiments, after pretreatment by any of the above methods, the feedstock contains cellulose, hemicellulose, soluble oligomers, simple sugars, lignins, volatiles and/or ash. The parameters of the pretreatment can be changed to vary the concentration of the components of the pretreated feedstock. For example, in some embodiments a pretreatment is chosen so that the concentration of hemicellulose and/or soluble oligomers is high and the concentration of lignins is low after pretreatment. Examples of parameters of the pretreatment include temperature, pressure, time, and pH.

In some embodiments, the parameters of the pretreatment are changed to vary the concentration of the components of the pretreated feedstock such that concentration of the components in the pretreated stock is optimal for fermentation with a microbe such as C. phytofermentans, Clostridium sp. Q.D, Clostridium phytofermentans Q.12, Clostridium phytofermentans Q.13, or genetically modified mutants thereof.

In some embodiments, the parameters of the pretreatment are changed such that concentration of accessible cellulose in the pretreated feedstock is 1%, 5%, 10%, 12%, 13%, 14%, 15%, 16%, 17%, 19%, 20%, 30%, 40% or 50%. In some embodiments, the parameters of the pretreatment are changed such that concentration of accessible cellulose in the pretreated feedstock is 5% to 30%. In some embodiments, the parameters of the pretreatment are changed such that concentration of accessible cellulose in the pretreated feedstock is 10% to 20%.

In some embodiments, the parameters of the pretreatment are changed such that concentration of hemicellulose in the pretreated feedstock is 1%, 5%, 10%, 12%, 13%, 14%, 15%, 16%, 17%, 19%, 20%, 21%, 22%, 23%, 24%, 25%, 26%, 27%, 28%, 29%, 30%, 40% or 50%. In some embodiments, the parameters of the pretreatment are changed such that concentration of hemicellulose in the pretreated feedstock is 5% to 40%. In some embodiments, the parameters of the pretreatment are changed such that concentration of hemicellulose in the pretreated feedstock is 10% to 30%.

In some embodiments, the parameters of the pretreatment are changed such that concentration of soluble oligomers in the pretreated feedstock is 1%, 10%, 15%, 20%, 25%, 30%, 35%, 40%, 45%, 50%, 55%, 60%, 65%, 70%, 75%, 80%, 85%, 90%, 95%, or 99%. Examples of soluble oligomers include, but are not limited to, cellobiose and xylobiose. In some embodiments, the parameters of the pretreatment are changed such that concentration of soluble oligomers in the pretreated feedstock is 30% to 90%. In some embodiments, the parameters of the pretreatment are changed such that concentration of soluble oligomers in the pretreated feedstock is 45% to 80%. In some embodiments, the parameters of the pretreatment are changed such that concentration of soluble oligomers in the pretreated feedstock is 45% to 80% and the soluble oligomers are primarily cellobiose and xylobiose.

In some embodiments, the parameters of the pretreatment are changed such that concentration of simple sugars in the pretreated feedstock is 1%, 5%, 10%, 12%, 13%, 14%, 15%, 16%, 17%, 19%, 20%, 30%, 40% or 50%. In some embodiments, the parameters of the pretreatment are changed such that concentration of simple sugars in the pretreated feedstock is 0% to 20%. In some embodiments, the parameters of the pretreatment are changed such that concentration of simple sugars in the pretreated feedstock is 0% to 5%. Examples of simple sugars include, but are not limited to, C5 and C6 monomers and dimers.

In some embodiments, the parameters of the pretreatment are changed such that concentration of lignins in the pretreated feedstock is 1%, 5%, 10%, 12%, 13%, 14%, 15%, 16%, 17%, 19%, 20%, 30%, 40% or 50%. In some embodiments, the parameters of the pretreatment are changed such that concentration of lignins in the pretreated feedstock is 0% to 20%. In some embodiments, the parameters of the pretreatment are changed such that concentration of lignins in the pretreated feedstock is 0% to 5%. In some embodiments, the parameters of the pretreatment are changed such that concentration of lignins in the pretreated feedstock is less than 1% to 2%. In some embodiments, the parameters of the pretreatment are changed such that the concentration of phenolics is minimized.

In some embodiments, the parameters of the pretreatment are changed such that concentration of furfural and low molecular weight lignins in the pretreated feedstock is less than 10%, 9%, 8%, 7%, 6%, 5%, 4%, 3%, 2%, or 1%. In some embodiments, the parameters of the pretreatment are changed such that concentration of furfural and low molecular weight lignins in the pretreated feedstock is less than 1% to 2%.

In some embodiments, the parameters of the pretreatment are changed such that concentration of accessible cellulose is 10% to 20%, the concentration of hemicellulose is 10% to 30%, the concentration of soluble oligomers is 45% to 80%, the concentration of simple sugars is 0% to 5%, and the concentration of lignins is 0% to 5% and the concentration of furfural and low molecular weight lignins in the pretreated feedstock is less than 1% to 2%.

In some embodiments, the parameters of the pretreatment are changed to obtain a high concentration of hemicellulose (e.g., 30%, 35%, 40%, 45%, 50%, 55%, 60%, 65%, 70% or higher) and a low concentration of lignins (e.g., 1%, 2%, 3%, 4%, 5%, 10%, 15%, 20%, 25%, or 30%). In some embodiments, the parameters of the pretreatment are changed to obtain a high concentration of hemicellulose and a low concentration of lignins such that concentration of the components in the pretreated stock is optimal for fermentation with a microbe such as C. phytofermentans, Clostridium sp. Q.D, Clostridium phytofermentans Q.12, Clostridium phytofermentans Q.13, or other mutagenized species of Clostridium.

Certain conditions of pretreatment can be modified prior to, or concurrently with, introduction of a fermentative microorganism into the feedstock. For example, pretreated feedstock can be cooled to a temperature which allows for growth of the microorganism(s). As another example, pH can be altered prior to, or concurrently with, addition of one or more microorganisms.

Alteration of the pH of a pretreated feedstock can be accomplished by washing the feedstock (e.g., with water) one or more times to remove an alkaline or acidic substance, or other substance used or produced during pretreatment. Washing can comprise exposing the pretreated feedstock to an equal volume of water 2, 3, 4, 5, 6, 7, 8, 9, 10, 11, 12, 13, 14, 15, 16, 17, 18, 19, 20, 21, 22, 23, 24, 25 or more times. In another embodiment, a pH modifier can be added. For example, an acid, a buffer, or a material that reacts with other materials present can be added to modulate the pH of the feedstock. In some embodiments, more than one pH modifier can be used, such as one or more bases, one or more bases with one or more buffers, one or more acids, one or more acids with one or more buffers, or one or more buffers. When more than one pH modifiers are utilized, they can be added at the same time or at different times. Other non-limiting exemplary methods for neutralizing feedstocks treated with alkaline substances have been described, for example in U.S. Pat. Nos. 4,048,341; 4,182,780; and 5,693,296.

In some embodiments, one or more acids can be combined, resulting in a buffer. Suitable acids and buffers that can be used as pH modifiers include any liquid or gaseous acid that is compatible with the microorganism. Non-limiting examples include peroxyacetic acid, sulfuric acid, lactic acid, citric acid, phosphoric acid, and hydrochloric acid. In some instances, the pH can be lowered to neutral pH or acidic pH, for example a pH of 7.0, 6.5, 6.0, 5.5, 5.0, 4.5, 4.0, or lower. In some embodiments, the pH is lowered and/or maintained within a range of about pH 4.5 to about 7.1, or about 4.5 to about 6.9, or about pH 5.0 to about 6.3, or about pH 5.5 to about 6.3, or about pH 6.0 to about 6.5, or about pH 5.5 to about 6.9 or about pH 6.2 to about 6.7.

In another embodiment, biomass can be pre-treated at an elevated temperature and/or pressure. In one embodiment biomass is pre treated at a temperature range of 20° C. to 400° C. In another embodiment biomass is pretreated at a temperature of about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C., 350° C., 400° C. or higher. In another embodiment, elevated temperatures are provided by the use of steam, hot water, or hot gases. In one embodiment steam can be injected into a biomass containing vessel. In another embodiment the steam, hot water, or hot gas can be injected into a vessel jacket such that it heats, but does not directly contact the biomass.

In another embodiment, a biomass can be treated at an elevated pressure. In one embodiment biomass is pre treated at a pressure range of about 1 psi to about 30 psi. In another embodiment biomass is pre treated at a pressure or about 1 psi, 2 psi, 3 psi, 4 psi, 5 psi, 6 psi, 7 psi, 8 psi, 9 psi, 10 psi, 12 psi, 15 psi, 18 psi, 20 psi, 22 psi, 24 psi, 26 psi, 28 psi, 30 psi or more. In some embodiments, biomass can be treated with elevated pressures by the injection of steam into a biomass containing vessel. In other embodiments, the biomass can be treated to vacuum conditions prior or subsequent to alkaline or acid treatment or any other treatment methods provided herein.

In one embodiment alkaline or acid pretreated biomass is washed (e.g. with water (hot or cold) or other solvent such as alcohol (e.g. ethanol)), pH neutralized with an acid, base, or buffering agent (e.g. phosphate, citrate, borate, or carbonate salt) or dried prior to fermentation. In one embodiment, the drying step can be performed under vacuum to increase the rate of evaporation of water or other solvents. Alternatively, or additionally, the drying step can be performed at elevated temperatures such as about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C. or more.

In some embodiments of the present invention, the pretreatment step includes a step of solids recovery. The solids recovery step can be during or after pretreatment (e.g., acid or alkali pretreatment), or before the drying step. In some embodiments, the solids recovery step provided by the methods of the present invention includes the use of a sieve, filter, screen, or a membrane for separating the liquid and solids fractions. In one embodiment a suitable sieve pore diameter size ranges from about 0.001 microns to 8 mm, such as about 0.005 microns to 3 mm or about 0.01 microns to 1 mm. In one embodiment a sieve pore size has a pore diameter of about 0.01 microns, 0.02 microns, 0.05 microns, 0.1 microns, 0.5 microns, 1 micron, 2 microns, 4 microns, 5 microns, 10 microns, 20 microns, 25 microns, 50 microns, 75 microns, 100 microns, 125 microns, 150 microns, 200 microns, 250 microns, 300 microns, 400 microns, 500 microns, 750 microns, 1 mm or more.

In some embodiments, biomass (e.g. corn stover) is processed or pretreated prior to fermentation. In one embodiment a method of pre-treatment includes but is not limited to, biomass particle size reduction, such as for example shredding, milling, chipping, crushing, grinding, or pulverizing. In some embodiments, biomass particle size reduction can include size separation methods such as sieving, or other suitable methods known in the art to separate materials based on size. In one embodiment size separation can provide for enhanced yields. In some embodiments, separation of finely shredded biomass (e.g. particles smaller than about 8 mm in diameter, such as, 8, 7.9, 7.7, 7.5, 7.3, 7, 6.9, 6.7, 6.5, 6.3, 6, 5.9, 5.7, 5.5, 5.3, 5, 4.9, 4.7, 4.5, 4.3, 4, 3.9, 3.7, 3.5, 3.3, 3, 2.9, 2.7, 2.5, 2.3, 2, 1.9, 1.7, 1.5, 1.3, 1, 0.9, 0.8, 0.7, 0.6, 0.5, 0.4, 0.3, 0.2, or 0.1 mm) from larger particles allows the recycling of the larger particles back into the size reduction process, thereby increasing the final yield of processed biomass. In one embodiment, a fermentative mixture is provided which comprises a pretreated lignocellulosic feedstock comprising less than about 50% of a lignin component present in the feedstock prior to pretreatment and comprising more than about 60% of a hemicellulose component present in the feedstock prior to pretreatment; and a microorganism capable of fermenting a five-carbon sugar, such as xylose, arabinose or a combination thereof, and a six-carbon sugar, such as glucose, galactose, mannose or a combination thereof. In some instances, pretreatment of the lignocellulosic feedstock comprises adding an alkaline substance which raises the pH to an alkaline level, for example NaOH. In some embodiments, NaOH is added at a concentration of about 0.5% to about 2% by weight of the feedstock. In other embodiments, pretreatment also comprises addition of a chelating agent. In some embodiments, the microorganism is a bacterium, such as a member of the genus Clostridium, for example Clostridium phytofermentans, Clostridium sp. Q.D, Clostridium phytofermentans Q.12 or Clostridium phytofermentans Q.13.

The present disclosure also provides a fermentative mixture comprising: a cellulosic feedstock pre-treated with an alkaline substance which maintains an alkaline pH, and at a temperature of from about 80° C. to about 120° C.; and a microorganism capable of fermenting a five-carbon sugar and a six-carbon sugar. In some instances, the five-carbon sugar is xylose, arabinose, or a combination thereof. In other instances, the six-carbon sugar is glucose, galactose, mannose, or a combination thereof. In some embodiments, the alkaline substance is NaOH. In some embodiments, NaOH is added at a concentration of about 0.5% to about 2% by weight of the feedstock. In some embodiments, the microorganism is a bacterium, such as a member of the genus Clostridium, for example Clostridium phytofermentans, Clostridium sp. Q.D, Clostridium phytofermentans Q.12 or Clostridium phytofermentans Q.13. In still other embodiments, the microorganism is genetically modified to enhance activity of one or more hydrolytic enzymes.

Further provided herein is a fermentative mixture comprising a cellulosic feedstock pre-treated with an alkaline substance which increases the pH to an alkaline level, at a temperature of from about 80° C. to about 120° C.; and a microorganism capable of uptake and fermentation of an oligosaccharide. In some embodiments the alkaline substance is NaOH. In some embodiments, NaOH is added at a concentration of about 0.5% to about 2% by weight of the feedstock. In some embodiments, the microorganism is a bacterium, such as a member of the genus Clostridium, for example Clostridium phytofermentans, Clostridium sp. Q.D, Clostridium phytofermentans Q.12 or Clostridium phytofermentans Q.13. In other embodiments, the microorganism is genetically modified to express or increase expression of an enzyme capable of hydrolyzing the oligosaccharide, a transporter capable of transporting the oligosaccharide, or a combination thereof.

Another aspect of the present disclosure provides a fermentative mixture comprising a cellulosic feedstock comprising cellulosic material from one or more sources, wherein said feedstock is pre-treated with a substance which increases the pH to an alkaline level, at a temperature of from about 80° C. to about 120° C.; and a microorganism capable of fermenting said cellulosic material from at least two different sources to produce a fermentation end-product at substantially a same yield coefficient. In some instances, the sources of cellulosic material are corn stover, bagasse, switchgrass or poplar. In some embodiments the alkaline substance is NaOH. In some embodiments, NaOH is added at a concentration of about 0.5% to about 2% by weight of the feedstock. In some embodiments, the microorganism is a bacterium, such as a member of the genus Clostridium, for example Clostridium phytofermentans, Clostridium sp. Q.D, Clostridium phytofermentans Q.12 or Clostridium phytofermentans Q.13.

In some embodiments, a process for simultaneous saccharification and fermentation of cellulosic solids from biomass into biofuel or another end-product is provided. In one embodiment the process comprises treating the biomass in a closed container with a microorganism under conditions where the microorganism produces saccharolytic enzymes sufficient to substantially convert the biomass into oligomers, monosaccharides and disaccharides. In one embodiment the organism subsequently converts the oligomers, monosaccharides and disaccharides into ethanol and/or another biofuel or product.

In an another embodiment, a process for saccharification and fermentation comprises treating the biomass in a container with the microorganism, and adding one or more enzymes before, concurrent or after contacting the biomass with the microorganism, wherein the enzymes added aid in the breakdown or detoxification of carbohydrates or lignocellulosic material.

In one embodiment, the bioconversion process comprises a separate hydrolysis and fermentation (SHF) process. In an SHF embodiment, the enzymes can be used under their optimal conditions regardless of the fermentation conditions and the organism is only required to ferment released sugars. In this embodiment, hydrolysis enzymes are externally added.

In another embodiment, the bioconversion process comprises a saccharification and fermentation (SSF) process. In an SSF embodiment, hydrolysis and fermentation take place in the same reactor under the same conditions.

In another embodiment, the bioconversion process comprises a consolidated bioprocess (CBP). In essence, CBP is a variation of SSF in which the enzymes are produced by the organism that carries out the fermentation. In this embodiment, enzymes can be both externally added enzymes and enzymes produced by the fermentative microbe. In this embodiment, biomass is partially hydrolyzed with externally added enzymes at their optimal condition, the slurry is then transferred to a separate tank in which the fermentative microbe (e.g. Clostridium phytofermentans, Clostridium sp. Q.D, Clostridium phytofermentans Q.12 or Clostridium phytofermentans Q.13) converts the hydrolyzed sugar into the desired product (e.g. fuel or chemical) and completes the hydrolysis of the residual cellulose and hemicellulose.

In one embodiment, pretreated biomass is partially hydrolyzed by externally added enzymes to reduce the viscosity. Hydrolysis occurs at the optimal pH and temperature conditions (e.g. pH 5.5, 50° C. for fungal cellulases). Hydrolysis time and enzyme loading can be adjusted such that conversion is limited to cellodextrins (soluble and insoluble) and hemicellulose oligomers. At the conclusion of the hydrolysis time, the resultant mixture can be subjected to fermentation conditions. For example, the resultant mixture can be pumped over time (fed batch) into a reactor containing a microorganism (e.g. Clostridium phytofermentans, Clostridium sp. Q.D, Clostridium phytofermentans Q.12 or Clostridium phytofermentans Q.13) and media. The microorganism can then produce endogenous enzymes to complete the hydrolysis into fermentable sugars (soluble oligomers) and convert those sugars into ethanol and/or other products in a production tank. The production tank can then be operated under fermentation optimal conditions (e.g. pH 6.5, 35° C.). In this way externally added enzyme is minimized due to operation under the enzyme's optimal conditions and due to a portion of the enzyme coming from C. phytofermentans.

In some embodiments, exogenous enzymes added include a xylanase, a hemicellulase, a glucanase or a glucosidase. In some embodiments, exogenous enzymes added do not include a xylanase, a hemicellulase, a glucanase or a glucosidase. In other embodiments, the amount of exogenous cellulase is greatly reduced, one-quarter or less of the amount normally added to a fermentation by a microorganism that cannot saccharify the biomass.

In one embodiment a second microorganism can be used to convert residual carbohydrates into a fermentation end-product. In one embodiment the Examples the second microorganism is a yeast such as Saccharomyces cerevisiae; a Clostridia species such as C. thermocellum, C. acetobutylicum, and C. cellovorans; or Zymomonas mobilis.

In one embodiment, a process of producing a biofuel or chemical product from a lignin-containing biomass is provided. In one embodiment the process comprises: 1) contacting the lignin-containing biomass with an aqueous alkaline solution at a concentration sufficient to hydrolyze at least a portion of the lignin-containing biomass; 2) neutralizing the treated biomass to a pH between 5 to 9 (e.g. 5.5, 6, 6.5, 7, 7.5, 8, 8.5, or 9); 3) treating the biomass in a closed container with a Clostridium microorganism, (such as Clostridium phytofermentans, a Clostidiom sp. Q.D, a Clostridium phytofermentans Q.13 or a Clostridium phytofermentans Q.12) under conditions wherein the Clostridium microorganism, optionally with the addition of one or more hydrolytic enzymes to the container, substantially converts the treated biomass into oligomers, monosaccharides and disaccharides, and/or biofuel or other fermentation end-product; and 4) optionally, introducing a culture of a second microorganism wherein the second microorganism is capable of substantially converting the oligomers, monosaccharides and disaccharides into biofuel.

Of various molecules typically found in biomass, cellulose is useful as a starting material for the production of fermentation end-products in methods and compositions described herein. Cellulose is one of the major components in plant cell wall. Cellulose is a linear condensation polymer consisting of D-anhydro glucopyranose joined together by β-1,4-linkage. The degree of polymerization ranges from 100 to 20,000. Adjacent cellulose molecules are coupled by extensive hydrogen bonds and van der Waals forces, resulting in a parallel alignment. The parallel sheet-like structure renders cellulose very stable.

Pretreatment can also include utilization of one or more strong cellulose swelling agents that facilitate disruption of the fiber structure and thus rendering the cellulosic material more amendable to saccharification and fermentation. Some considerations have been given in selecting an efficient method of swelling for various cellulosic material: 1) the hydrogen bonding fraction; 2) solvent molar volume; 3) the cellulose structure. The width and distribution of voids (between the chains of linear cellulosic polymer) are important as well. It is known that the swelling is more pronounced in the presence of electrostatic repulsion, provided by alkali solution or ionic surfactants. Of course, with respect to utilization of any of the methods disclosed herein, conditioning of a biomass can be concurrent to contact with an organism that is capable of saccharification and fermentation. In addition, other examples describing the pretreatment of lignocellulosic biomass have been published as U.S. Pat. Nos. 4,304,649, 5,366,558, 5,411,603, and 5,705,369.

Biomass Processing

Described herein are compositions and methods allowing saccharification and fermentation to one or more industrially useful fermentation end-products. Saccharification includes conversion of long-chain sugar polymers, such as cellulose, to monosaccharides, disaccharides, trisaccharides, and oligosaccharides of up to about seven monomer units, as well as similar sized chains of sugar derivatives and combinations of sugars and sugar derivatives. The chain-length for saccharides may be longer (e.g. 8, 9, 10, 11, 12, 13, 14, 15, 16, 17, 18, 19, or 20 monomer units or more) and or shorter (e.g. 1, 2, 3, 4, 5, 6 monomer units). As used herein, “directly processing” means that an organism is capable of both hydrolyzing biomass and fermenting without the need for conditioning the biomass, such as subjecting the biomass to chemical, heat, enzymatic treatment or combinations thereof.

Methods and compositions described herein contemplate utilizing fermentation process for extracting industrially useful fermentation end-products from biomass. The term “fermentation” as used herein has its ordinary meaning as known to those skilled in the art and may include culturing of a microorganism or group of microorganisms in or on a suitable medium for the microorganisms. The microorganisms can be aerobes, anaerobes, facultative anaerobes, heterotrophs, autotrophs, photoautotrophs, photoheterotrophs, chemoautotrophs, and/or chemoheterotrophs. The cellular activity, including cell growth can be growing aerobic, microaerophilic, or anaerobic. The cells can be in any phase of growth, including lag (or conduction), exponential, transition, stationary, death, dormant, vegetative, sporulating, etc.

Organisms disclosed herein can be incorporated into methods and compositions of the inventon so as to enhance fermentation end-product yield and/or rate of production. One example of such an organism is Clostridium phytofermentans (“C. phytofermentans”), which can simultaneously hydrolyze and ferment lignocellulose biomass. Furthermore, C. phytofermentans is capable of fermenting hexose (C6) and pentose (C5) polysaccharides. In addition, C. phytofermentans is capable of acting directly on lignocellulosic biomass without any pretreatment. Other examples include Clostridium sp. Q.D, or mutagenized species of Clostridium phytofermentans, such as Clostridium Q.12, Clostridium phytofermentans Q.13, or a genetically-modified species of C. phytofermentans.

A genetically modified organism of the invention can be further modified to heterologously express one or more cellulases, or further modified to enhance expression of one or more endogenous cellulases, thereby further enhancing the hydrolysis of biomass. Products of the invention include a product for production of a biofuel comprising: a carbonaceous biomass, a microorganism that is capable of direct hydrolysis and fermentation of said biomass, wherein said microorganism is modified to provide enhanced activity of one or more cellulases.

A genetically modified organism of the invention can be further modified to heterologously express one or more antioxidants (e.g. catalase, superoxide dismutase or glutathione peroxidase), thereby further enhancing the hydrolysis of biomass. Such microorganisms are further described in International Application Serial Number PCT/US2010/059962, disclosing expression of antioxidants in microorganisms to enhance production of fermentation end-products, and which is herein incorporated by reference in its entirety. Products of the invention include a product for production of a biofuel or chemical product comprising: a carbonaceous biomass, a microorganism that is capable of direct hydrolysis and fermentation of said biomass, wherein said microorganism is modified to provide enhanced activity of one or more antioxidants.

In one embodiment, a genetically modified microorganism of the invention can be modified to heterologously express one or more enzymes to enchance the production of 1,4 diacid (succinic, fumaric and malic). Example enzymes include, but are not limited to, proteins encoded by the genes Cphy_{—}0409, Cphy_{—}0007, Cphy_{—}0008, Cphy_{—}3299 and Cphy_{—}3885.

In one embodiment, a genetically modified microorganism of the invention can be modified to heterologously express one or more enzymes to enchance the production 2,5 furan dicarboxylic acid.

In one embodiment, a genetically modified microorganism of the invention can be modified to heterologously express one or more enzymes to enchance the production of aspartic acid. Example enzymes include, but are not limited to, aspartate aminotransferase, aspartate dehydrogenase, and aspartase (L-aspartate ammonialyase).

In one embodiment, a genetically modified microorganism of the invention can be modified to heterologously express one or more enzymes to enchance the production of glucaric acid.

In one embodiment, a genetically modified microorganism of the invention can be modified to heterologously express one or more enzymes to enchance the production of glutamic acid. Examples of enzymes include, but are not limited to, D-glutamate ligase and glutamine synthase.

In one embodiment, a genetically modified microorganism of the invention can be modified to heterologously express one or more enzymes to enchance the production of itaconic acid. Examples of enzymes include, but are not limited to, cis-aconitate decarboxylase, cis-aconitate decarboxylase, citrate synthase, aconitase, cis-aconitic acid decarboxylase (itaconate-forming), cis-aconitic acid decarboxylase (citraconate-forming), citraconate isomerase, mitochondrial dicarboxylate-tricarboxylate antiporter, mitochondrial tricarboxylate transporter, dicarboxylate transporter, an 2-methylcitrate dehydratase.

In one embodiment, a genetically modified microorganism of the invention can be modified to heterologously express one or more enzymes to enchance the production of 3-hydroxybutyrolactone. Examples include, but are not limited to,

malonate-semialdehyde dehydrogenase, aldehyde dehydrogenase, 3-hydroxyisobutyrate dehydrogenase, beta-alanine/pyruvate aminotransferase, and alanine 2,3-aminomutase activity.

In one embodiment, a genetically modified microorganism of the invention can be modified to heterologously express one or more enzymes to enchance the production of glycerol. Example enzymes include, but are not limited to, glycerol-3-phosphate, dihydroxyacetone kinase EC2.7.1.29, alcohol dehydrogenase, GldA (glycerol dehydrogenase and related enzymes), NAD (P)H-dependent glycerol-3-phosphate dehydrogenase, FAD-dependent oxidoreductase, and glycerol 3-phosphatase (Gpp p).

In one embodiment, a genetically modified microorganism of the invention can be modified to heterologously express one or more enzymes to enchance the production sorbitol. Example enzymes include, but are not limited to, D-sorbitol dehydrogenases (GutB) and D-sorbitol-6-phosphate dehydrogenase-encoding gene (gutF).

In one embodiment, a genetically modified microorganism of the invention can be modified to heterologously express one or more enzymes to enchance the production of xylitol/arabinitol. Example enzymes include, but are not limited to, D-xylulose reductase, D-arabitol 4-dehydrogenase, NAD-dependent xylitol dehydrogenase, and xylitol-phosphate dehydrogenase, D-arabitol-phosphate dehydrogenase.

In one embodiment, a genetically modified microorganism of the invention can be modified to heterologously express one or more enzymes to enchance the production of butanediol. Example enzymes include, but are not limited to, acetoin reductase/2,3-butanediol dehydrogenases (AR/BDH), acetolactate synthase, acetolactate, L-butanediol dehydrogenase, acetoin racemase, and acetoin reductase.

In one embodiment, a genetically modified microorganism of the invention can be modified to heterologously express one or more enzymes to enchance the production of butanol. Example enzymes include, but are not limited to, acetyl-CoA acetyl transferase, 3-hydroxybutyryl-CoA dehydrogenase, crotonase, butyryl-CoA dehydrogenase, butyraldehyde dehydrogenase, butanol dehydrogenase, 3-hydroxybutyryl-CoA dehydrogenase, crotonase, butyraldehyde dehydrogenase, 3-oxoacyl-(acyl-carrier-protein) synthase 2, and iron-containing alcohol dehydrogenase.

In one embodiment, a genetically modified microorganism of the invention can be modified to heterologously express one or more enzymes to enchance the production of terpenes and terpenoids (or isoprenoids). Example enzymes include, but are not limited to, DOXP synthase (Dxs), DOXP reductase (Dxr, IspC), 4-diphosphocytidyl-2-C-methyl-D-erythritol synthase (YgbP, IspD), 4-diphosphocytidyl-2-C-methyl-D-erythritol kinase (YchB, IspE), 2-C-methyl-D-erythritol 2,4-cyclodiphosphate synthase (YgbB, IspF), HMB-PP synthase (GcpE, IspG), and HMB-PP reductase (LytB, IspH).

Any of the microorganisms described herein can be used for fermentation. For example, a strain for fermentation is Clostridium phytofermentans. The biomass includes, but is not limited to, plant material, carbonaceous biomass or any material containing cellulosic, hemicellulosic, and/or lignocellulosic material. Examples of such biomass can be pectin, starch, inulin, fructans, glucans, corn, corn stover, sugar cane, grasses, switch grass, bamboo and algae.

Co-Culture Methods and Compositions

Methods of the invention can also included co-culture with an organism that naturally produces or is genetically modified to produce one or more enzymes, such as hydrolytic enzymes (such as cellulase(s), hemicellulase(s), or pectinases etc.), antioxidants (such as. catalase, superoxide dismutase or glutathione peroxidase), or enzymes listed in FIGS. 1 through 14. A culture medium containing such an organism can be contacted with biomass (e.g., in a bioreactor) prior to, concurrent with, or subsequent to contact with a second organism. In one embodiment a first organism produces an enzyme while a second organism saccharifies and ferments C5 and C6 sugars. Mixtures of microorganisms can be provided as solid mixtures (e.g., freeze-dried mixtures), or as liquid dispersions of the microorganisms, and grown in co-culture with a second microorganism. Co-culture methods capable of use with the present invention are known, such as those disclosed in U.S. Pat. Application No. 20070178569.

Fermentation End-Product

The term “fuel” or “biofuel” as used herein has its ordinary meaning as known to those skilled in the art and can include one or more compounds suitable as liquid fuels, gaseous fuels, biodiesel fuels (long-chain alkyl(methyl, propyl or ethyl) esters), heating oils (hydrocarbons in the 14-20 carbon range), reagents, chemical feedstocks and includes, but is not limited to, hydrocarbons (both light and heavy), hydrogen, methane, hydroxy compounds such as alcohols (e.g. ethanol, butanol, propanol, methanol, etc.), and carbonyl compounds such as aldehydes and ketones (e.g. acetone, formaldehyde, 1-propanal, etc.).

The terms “fermentation end-product” or “end-product” as used herein has its ordinary meaning as known to those skilled in the art and can include one or more biofuels, chemical additives, processing aids, food additives, organic acids (e.g. acetic, lactic, formic, citric acid etc.), derivatives of organic acids such as esters (e.g. wax esters, glycerides, etc.) or other functional compounds. These end-products include, but are not limited to, an alcohol, ethanol, butanol, methanol, 1,2-propanediol, 1,3-propanediol, lactic acid, formic acid, acetic acid, succinic acid, pyruvic acid, enzymes such as cellulases, polysaccharases, lipases, proteases, ligninases, and hemicellulases and can be present as a pure compound, a mixture, or an impure or diluted form.

Production of various fermentation end-products can be made or enhanced through saccharification and fermentation using enzyme-enhancing products and processes. Examples of end-products include but are not limited to 1,4 diacid (succinic, fumaric and malic), 2,5 furan dicarboxylic acid, 3-hydroxy propionic acid, aspartic acid, glucaric acid, glutamic acid, itaconic acid, levulinic acid, 3-hydroxybutyrolactone, glycerol, sorbitol, xylitol/arabitol, butanediol, butanol, isopentenyl diphosphate, methane, methanol, ethane, ethene, ethanol, n-propane, 1-propene, 1-propanol, propanal, acetone, propionate, n-butane, 1-butene, 1-butanol, butanal, butanoate, isobutanal, isobutanol, 2-methylbutanal, 2-methylbutanol, 3-methylbutanal, 3-methylbutanol, 2-butene, 2-butanol, 2-butanone, 2,3-butanediol, 3-hydroxy-2-butanone, 2,3-butanedione, ethylbenzene, ethenylbenzene, 2-phenylethanol, phenylacetaldehyde, 1-phenylbutane, 4-phenyl-1-butene, 4-phenyl-2-butene, 1-phenyl-2-butene, 1-phenyl-2-butanol, 4-phenyl-2-butanol, 1-phenyl-2-butanone, 4-phenyl-2-butanone, 1-phenyl-2,3-butandiol, 1-phenyl-3-hydroxy-2-butanone, 4-phenyl-3-hydroxy-2-butanone, 1-phenyl-2,3-butanedione, n-pentane, ethylphenol, ethenylphenol, 2-(4-hydroxyphenyl)ethanol, 4-hydroxyphenylacetaldehyde, 1-(4-hydroxyphenyl)butane, 4-(4-hydroxyphenyl)-1-butene, 4-(4-hydroxyphenyl)-2-butene, 1-(4-hydroxyphenyl)-1-butene, 1-(4-hydroxyphenyl)-2-butanol, 4-(4-hydroxyphenyl)-2-butanol, 1-(4-hydroxyphenyl)-2-butanone, 4-(4-hydroxyphenyl)-2-butanone, 1-(4-hydroxyphenyl)-2,3-butandiol, 1-(4-hydroxyphenyl)-3-hydroxy-2-butanone, 4-(4-hydroxyphenyl)-3-hydroxy-2-butanone, 1-(4-hydroxyphenyl)-2,3-butanonedione, indolylethane, indolylethene, 2-(indole-3-)ethanol, n-pentane, 1-pentene, 1-pentanol, pentanal, pentanoate, 2-pentene, 2-pentanol, 3-pentanol, 2-pentanone, 3-pentanone, 4-methylpentanal, 4-methylpentanol, 2,3-pentanediol, 2-hydroxy-3-pentanone, 3-hydroxy-2-pentanone, 2,3-pentanedione, 2-methylpentane, 4-methyl-1-pentene, 4-methyl-2-pentene, 4-methyl-3-pentene, 4-methyl-2-pentanol, 2-methyl-3-pentanol, 4-methyl-2-pentanone, 2-methyl-3-pentanone, 4-methyl-2,3-pentanediol, 4-methyl-2-hydroxy-3-pentanone, 4-methyl-3-hydroxy-2-pentanone, 4-methyl-2,3-pentanedione, 1-phenylpentane, 1-phenyl-1-pentene, 1-phenyl-2-pentene, 1-phenyl-3-pentene, 1-phenyl-2-pentanol, 1-phenyl-3-pentanol, 1-phenyl-2-pentanone, 1-phenyl-3-pentanone, 1-phenyl-2,3-pentanediol, 1-phenyl-2-hydroxy-3-pentanone, 1-phenyl-3-hydroxy-2-pentanone, 1-phenyl-2,3-pentanedione, 4-methyl-1-phenylpentane, 4-methyl-1-phenyl-1-pentene, 4-methyl-1-phenyl-2-pentene, 4-methyl-1-phenyl-3-pentene, 4-methyl-1-phenyl-3-pentanol, 4-methyl-1-phenyl-2-pentanol, 4-methyl-1-phenyl-3-pentanone, 4-methyl-1-phenyl-2-pentanone, 4-methyl-1-phenyl-2,3-pentanediol, 4-methyl-1-phenyl-2,3-pentanedione, 4-methyl-1-phenyl-3-hydroxy-2-pentanone, 4-methyl-1-phenyl-2-hydroxy-3-pentanone, 1-(4-hydroxyphenyl)pentane, 1-(4-hydroxyphenyl)-1-pentene, 1-(4-hydroxyphenyl)-2-pentene, 1-(4-hydroxyphenyl)-3-pentene, 1-(4-hydroxyphenyl)-2-pentanol, 1-(4-hydroxyphenyl)-3-pentanol, 1-(4-hydroxyphenyl)-2-pentanone, 1-(4-hydroxyphenyl)-3-pentanone, 1-(4-hydroxyphenyl)-2,3-pentanediol, 1-(4-hydroxyphenyl)-2-hydroxy-3-pentanone, 1-(4-hydroxyphenyl)-3-hydroxy-2-pentanone, 1-(4-hydroxyphenyl)-2,3-pentanedione, 4-methyl-1-(4-hydroxyphenyl)pentane, 4-methyl-1-(4-hydroxyphenyl)-2-pentene, 4-methyl-1-(4-hydroxyphenyl)-3-pentene, 4-methyl-1-(4-hydroxyphenyl)-1-pentene, 4-methyl-1-(4-hydroxyphenyl)-3-pentanol, 4-methyl-1-(4-hydroxyphenyl)-2-pentanol, 4-methyl-1-(4-hydroxyphenyl)-3-pentanone, 4-methyl-1-(4-hydroxyphenyl)-2-pentanone, 4-methyl-1-(4-hydroxyphenyl)-2,3-pentanediol, 4-methyl-1-(4-hydroxyphenyl)-2,3-pentanedione, 4-methyl-1-(4-hydroxyphenyl)-3-hydroxy-2-pentanone, 4-methyl-1-(4-hydroxyphenyl)-2-hydroxy-3-pentanone, 1-indole-3-pentane, 1-(indole-3)-1-pentene, 1-(indole-3)-2-pentene, 1-(indole-3)-3-pentene, 1-(indole-3)-2-pentanol, 1-(indole-3)-3-pentanol, 1-(indole-3)-2-pentanone, 1-(indole-3)-3-pentanone, 1-(indole-3)-2,3-pentanediol, 1-(indole-3)-2-hydroxy-3-pentanone, 1-(indole-3)-3-hydroxy-2-pentanone, 1-(indole-3)-2,3-pentanedione, 4-methyl-1-(indole-3-)pentane, 4-methyl-1-(indole-3)-2-pentene, 4-methyl-1-(indole-3)-3-pentene, 4-methyl-1-(indole-3)-1-pentene, 4-methyl-2-(indole-3)-3-pentanol, 4-methyl-1-(indole-3)-2-pentanol, 4-methyl-1-(indole-3)-3-pentanone, 4-methyl-1-(indole-3)-2-pentanone, 4-methyl-1-(indole-3)-2,3-pentanediol, 4-methyl-1-(indole-3)-2,3-pentanedione, 4-methyl-1-(indole-3)-3-hydroxy-2-pentanone, 4-methyl-1-(indole-3)-2-hydroxy-3-pentanone, n-hexane, 1-hexene, 1-hexanol, hexanal, hexanoate, 2-hexene, 3-hexene, 2-hexanol, 3-hexanol, 2-hexanone, 3-hexanone, 2,3-hexanediol, 2,3-hexanedione, 3,4-hexanediol, 3,4-hexanedione, 2-hydroxy-3-hexanone, 3-hydroxy-2-hexanone, 3-hydroxy-4-hexanone, 4-hydroxy-3-hexanone, 2-methylhexane, 3-methylhexane, 2-methyl-2-hexene, 2-methyl-3-hexene, 5-methyl-1-hexene, 5-methyl-2-hexene, 4-methyl-1-hexene, 4-methyl-2-hexene, 3-methyl-3-hexene, 3-methyl-2-hexene, 3-methyl-1-hexene, 2-methyl-3-hexanol, 5-methyl-2-hexanol, 5-methyl-3-hexanol, 2-methyl-3-hexanone, 5-methyl-2-hexanone, 5-methyl-3-hexanone, 2-methyl-3,4-hexanediol, 2-methyl-3,4-hexanedione, 5-methyl-2,3-hexanediol, 5-methyl-2,3-hexanedione, 4-methyl-2,3-hexanediol, 4-methyl-2,3-hexanedione, 2-methyl-3-hydroxy-4-hexanone, 2-methyl-4-hydroxy-3-hexanone, 5-methyl-2-hydroxy-3-hexanone, 5-methyl-3-hydroxy-2-hexanone, 4-methyl-2-hydroxy-3-hexanone, 4-methyl-3-hydroxy-2-hexanone, 2,5-dimethylhexane, 2,5-dimethyl-2-hexene, 2,5-dimethyl-3-hexene, 2,5-dimethyl-3-hexanol, 2,5-dimethyl-3-hexanone, 2,5-dimethyl-3,4-hexanediol, 2,5-dimethyl-3,4-hexanedione, 2,5-dimethyl-3-hydroxy-4-hexanone, 5-methyl-1-phenylhexane, 4-methyl-1-phenylhexane, 5-methyl-1-phenyl-1-hexene, 5-methyl-1-phenyl-2-hexene, 5-methyl-1-phenyl-3-hexene, 4-methyl-1-phenyl-1-hexene, 4-methyl-1-phenyl-2-hexene, 4-methyl-1-phenyl-3-hexene, 5-methyl-1-phenyl-2-hexanol, 5-methyl-1-phenyl-3-hexanol, 4-methyl-1-phenyl-2-hexanol, 4-methyl-1-phenyl-3-hexanol, 5-methyl-1-phenyl-2-hexanone, 5-methyl-1-phenyl-3-hexanone, 4-methyl-1-phenyl-2-hexanone, 4-methyl-1-phenyl-3-hexanone, 5-methyl-1-phenyl-2,3-hexanediol, 4-methyl-1-phenyl-2,3-hexanediol, 5-methyl-1-phenyl-3-hydroxy-2-hexanone, 5-methyl-1-phenyl-2-hydroxy-3-hexanone, 4-methyl-1-phenyl-3-hydroxy-2-hexanone, 4-methyl-1-phenyl-2-hydroxy-3-hexanone, 5-methyl-1-phenyl-2,3-hexanedione, 4-methyl-1-phenyl-2,3-hexanedione, 4-methyl-1-(4-hydroxyphenyl)hexane, 5-methyl-1-(4-hydroxyphenyl)-1-hexene, 5-methyl-1-(4-hydroxyphenyl)-2-hexene, 5-methyl-1-(4-hydroxyphenyl)-3-hexene, 4-methyl-1-(4-hydroxyphenyl)-1-hexene, 4-methyl-1-(4-hydroxyphenyl)-2-hexene, 4-methyl-1-(4-hydroxyphenyl)-3-hexene, 5-methyl-1-(4-hydroxyphenyl)-2-hexanol, 5-methyl-1-(4-hydroxyphenyl)-3-hexanol, 4-methyl-1-(4-hydroxyphenyl)-2-hexanol, 4-methyl-1-(4-hydroxyphenyl)-3-hexanol, 5-methyl-1-(4-hydroxyphenyl)-2-hexanone, 5-methyl-1-(4-hydroxyphenyl)-3-hexanone, 4-methyl-1-(4-hydroxyphenyl)-2-hexanone, 4-methyl-1-(4-hydroxyphenyl)-3-hexanone, 5-methyl-1-(4-hydroxyphenyl)-2,3-hexanediol, 4-methyl-1-(4-hydroxyphenyl)-2,3-hexanediol, 5-methyl-1-(4-hydroxyphenyl)-3-hydroxy-2-hexanone, 5-methyl-1-(4-hydroxyphenyl)-2-hydroxy-3-hexanone, 4-methyl-1-(4-hydroxyphenyl)-3-hydroxy-2-hexanone, 4-methyl-1-(4-hydroxyphenyl)-2-hydroxy-3-hexanone, 5-methyl-1-(4-hydroxyphenyl)-2,3-hexanedione, 4-methyl-1-(4-hydroxyphenyl)-2,3-hexanedione, 4-methyl-1-(indole-3-)hexane, 5-methyl-1-(indole-3)-1-hexene, 5-methyl-1-(indole-3)-2-hexene, 5-methyl-1-(indole-3)-3-hexene, 4-methyl-1-(indole-3)-1-hexene, 4-methyl-1-(indole-3)-2-hexene, 4-methyl-1-(indole-3)-3-hexene, 5-methyl-1-(indole-3)-2-hexanol, 5-methyl-1-(indole-3)-3-hexanol, 4-methyl-1-(indole-3)-2-hexanol, 4-methyl-1-(indole-3)-3-hexanol, 5-methyl-1-(indole-3)-2-hexanone, 5-methyl-1-(indole-3)-3-hexanone, 4-methyl-1-(indole-3)-2-hexanone, 4-methyl-1-(indole-3)-3-hexanone, 5-methyl-1-(indole-3)-2,3-hexanediol, 4-methyl-1-(indole-3)-2,3-hexanediol, 5-methyl-1-(indole-3)-3-hydroxy-2-hexanone, 5-methyl-1-(indole-3)-2-hydroxy-3-hexanone, 4-methyl-1-(indole-3)-3-hydroxy-2-hexanone, 4-methyl-1-(indole-3)-2-hydroxy-3-hexanone, 5-methyl-1-(indole-3)-2,3-hexanedione, 4-methyl-1-(indole-3)-2,3-hexanedione, n-heptane, 1-heptene, 1-heptanol, heptanal, heptanoate, 2-heptene, 3-heptene, 2-heptanol, 3-heptanol, 4-heptanol, 2-heptanone, 3-heptanone, 4-heptanone, 2,3-heptanediol, 2,3-heptanedione, 3,4-heptanediol, 3,4-heptanedione, 2-hydroxy-3-heptanone, 3-hydroxy-2-heptanone, 3-hydroxy-4-heptanone, 4-hydroxy-3-heptanone, 2-methylheptane, 3-methylheptane, 6-methyl-2-heptene, 6-methyl-3-heptene, 2-methyl-3-heptene, 2-methyl-2-heptene, -methyl-2-heptene, -methyl-3-heptene, 3-methyl-3-heptene, 2-methyl-3-heptanol, 2-methyl-4-heptanol, 6-methyl-3-heptanol, 5-methyl-3-heptanol, 3-methyl-4-heptanol, 2-methyl-3-heptanone, 2-methyl-4-heptanone, 6-methyl-3-heptanone, 5-methyl-3-heptanone, 3-methyl-4-heptanone, 2-methyl-3,4-heptanediol, 2-methyl-3,4-heptanedione, 6-methyl-3,4-heptanediol, 6-methyl-3,4-heptanedione, 5-methyl-3,4-heptanediol, 5-methyl-3,4-heptanedione, 2-methyl-3-hydroxy-4-heptanone, 2-methyl-4-hydroxy-3-heptanone, 6-methyl-3-hydroxy-4-heptanone, 6-methyl-4-hydroxy-3-heptanone, 5-methyl-3-hydroxy-4-heptanone, 5-methyl-4-hydroxy-3-heptanone, 2,6-dimethylheptane, 2,5-dimethylheptane, 2,6-dimethyl-2-heptene, 2,6-dimethyl-3-heptene, 2,5-dimethyl-2-heptene, 2,5-dimethyl-3-heptene, 3,6-dimethyl-3-heptene, 2,6-dimethyl-3-heptanol, 2,6-dimethyl-4-heptanol, 2,5-dimethyl-3-heptanol, 2,5-dimethyl-4-heptanol, 2,6-dimethyl-3,4-heptanediol, 2,6-dimethyl-3,4-heptanedione, 2,5-dimethyl-3,4-heptanediol, 2,5-dimethyl-3,4-heptanedione, 2,6-dimethyl-3-hydroxy-4-heptanone, 2,6-dimethyl-4-hydroxy-3-heptanone, 2,5-dimethyl-3-hydroxy-4-heptanone, 2,5-dimethyl-4-hydroxy-3-heptanone, n-octane, 1-octene, 2-octene, 1-octanol, octanal, octanoate, 3-octene, 4-octene, 4-octanol, 4-octanone, 4,5-octanediol, 4,5-octanedione, 4-hydroxy-5-octanone, 2-methyloctane, 2-methyl-3-octene, 2-methyl-4-octene, 7-methyl-3-octene, 3-methyl-3-octene, 3-methyl-4-octene, 6-methyl-3-octene, 2-methyl-4-octanol, 7-methyl-4-octanol, 3-methyl-4-octanol, 6-methyl-4-octanol, 2-methyl-4-octanone, 7-methyl-4-octanone, 3-methyl-4-octanone, 6-methyl-4-octanone, 2-methyl-4,5-octanediol, 2-methyl-4,5-octanedione, 3-methyl-4,5-octanediol, 3-methyl-4,5-octanedione, 2-methyl-4-hydroxy-5-octanone, 2-methyl-5-hydroxy-4-octanone, 3-methyl-4-hydroxy-5-octanone, 3-methyl-5-hydroxy-4-octanone, 2,7-dimethyloctane, 2,7-dimethyl-3-octene, 2,7-dimethyl-4-octene, 2,7-dimethyl-4-octanol, 2,7-dimethyl-4-octanone, 2,7-dimethyl-4,5-octanediol, 2,7-dimethyl-4,5-octanedione, 2,7-dimethyl-4-hydroxy-5-octanone, 2,6-dimethyloctane, 2,6-dimethyl-3-octene, 2,6-dimethyl-4-octene, 3,7-dimethyl-3-octene, 2,6-dimethyl-4-octanol, 3,7-dimethyl-4-octanol, 2,6-dimethyl-4-octanone, 3,7-dimethyl-4-octanone, 2,6-dimethyl-4,5-octanediol, 2,6-dimethyl-4,5-octanedione, 2,6-dimethyl-4-hydroxy-5-octanone, 2,6-dimethyl-5-hydroxy-4-octanone, 3,6-dimethyloctane, 3,6-dimethyl-3-octene, 3,6-dimethyl-4-octene, 3,6-dimethyl-4-octanol, 3,6-dimethyl-4-octanone, 3,6-dimethyl-4,5-octanediol, 3,6-dimethyl-4,5-octanedione, 3,6-dimethyl-4-hydroxy-5-octanone, n-nonane, 1-nonene, 1-nonanol, nonanal, nonanoate, 2-methylnonane, 2-methyl-4-nonene, 2-methyl-5-nonene, 8-methyl-4-nonene, 2-methyl-5-nonanol, 8-methyl-4-nonanol, 2-methyl-5-nonanone, 8-methyl-4-nonanone, 8-methyl-4,5-nonanediol, 8-methyl-4,5-nonanedione, 8-methyl-4-hydroxy-5-nonanone, 8-methyl-5-hydroxy-4-nonanone, 2,8-dimethylnonane, 2,8-dimethyl-3-nonene, 2,8-dimethyl-4-nonene, 2,8-dimethyl-5-nonene, 2,8-dimethyl-4-nonanol, 2,8-dimethyl-5-nonanol, 2,8-dimethyl-4-nonanone, 2,8-dimethyl-5-nonanone, 2,8-dimethyl-4,5-nonanediol, 2,8-dimethyl-4,5-nonanedione, 2,8-dimethyl-4-hydroxy-5-nonanone, 2,8-dimethyl-5-hydroxy-4-nonanone, 2,7-dimethylnonane, 3,8-dimethyl-3-nonene, 3,8-dimethyl-4-nonene, 3,8-dimethyl-5-nonene, 3,8-dimethyl-4-nonanol, 3,8-dimethyl-5-nonanol, 3,8-dimethyl-4-nonanone, 3,8-dimethyl-5-nonanone, 3,8-dimethyl-4,5-nonanediol, 3,8-dimethyl-4,5-nonanedione, 3,8-dimethyl-4-hydroxy-5-nonanone, 3,8-dimethyl-5-hydroxy-4-nonanone, n-decane, 1-decene, 1-decanol, decanoate, 2,9-dimethyldecane, 2,9-dimethyl-3-decene, 2,9-dimethyl-4-decene, 2,9-dimethyl-5-decanol, 2,9-dimethyl-5-decanone, 2,9-dimethyl-5,6-decanediol, 2,9-dimethyl-6-hydroxy-5-decanone, 2,9-dimethyl-5,6-decanedionen-undecane, 1-undecene, 1-undecanol, undecanal. undecanoate, n-dodecane, 1-dodecene, 1-dodecanol, dodecanal, dodecanoate, n-dodecane, 1-decadecene, n-tridecane, 1-tridecene, 1-tridecanol, tridecanal, tridecanoate, n-tetradecane, 1-tetradecene, 1-tetradecanol, tetradecanal, tetradecanoate, n-pentadecane, 1-pentadecene, 1-pentadecanol, pentadecanal, pentadecanoate, n-hexadecane, 1-hexadecene, 1-hexadecanol, hexadecanal, hexadecanoate, n-heptadecane, 1-heptadecene, 1-heptadecanol, heptadecanal, heptadecanoate, n-octadecane, 1-octadecene, 1-octadecanol, octadecanal, octadecanoate, n-nonadecane, 1-nonadecene, 1-nonadecanol, nonadecanal, nonadecanoate, eicosane, 1-eicosene, 1-eicosanol, eicosanal, eicosanoate, 3-hydroxy propanal, 1,3-propanediol, 4-hydroxybutanal, 1,4-butanediol, 3-hydroxy-2-butanone, 2,3-butandiol, 1,5-pentane diol, homocitrate, homoisocitorate, b-hydroxy adipate, glutarate, glutarsemialdehyde, glutaraldehyde, 2-hydroxy-1-cyclopentanone, 1,2-cyclopentanediol, cyclopentanone, cyclopentanol, (S)-2-acetolactate, (R)-2,3-Dihydroxy-isovalerate, 2-oxoisovalerate, isobutyryl-CoA, isobutyrate, isobutyraldehyde, 5-amino pentaldehyde, 1,10-diaminodecane, 1,10-diamino-5-decene, 1,10-diamino-5-hydroxydecane, 1,10-diamino-5-decanone, 1,10-diamino-5,6-decanediol, 1,10-diamino-6-hydroxy-5-decanone, phenylacetoaldehyde, 1,4-diphenylbutane, 1,4-diphenyl-1-butene, 1,4-diphenyl-2-butene, 1,4-diphenyl-2-butanol, 1,4-diphenyl-2-butanone, 1,4-diphenyl-2,3-butanediol, 1,4-diphenyl-3-hydroxy-2-butanone, 1-(4-hydeoxyphenyl)-4-phenylbutane, 1-(4-hydeoxyphenyl)-4-phenyl-1-butene, 1-(4-hydeoxyphenyl)-4-phenyl-2-butene, 1-(4-hydeoxyphenyl)-4-phenyl-2-butanol, 1-(4-hydeoxyphenyl)-4-phenyl-2-butanone, 1-(4-hydeoxyphenyl)-4-phenyl-2,3-butanediol, 1-(4-hydeoxyphenyl)-4-phenyl-3-hydroxy-2-butanone, 1-(indole-3)-4-phenylbutane, 1-(indole-3)-4-phenyl-1-butene, 1-(indole-3)-4-phenyl-2-butene, 1-(indole-3)-4-phenyl-2-butanol, 1-(indole-3)-4-phenyl-2-butanone, 1-(indole-3)-4-phenyl-2,3-butanediol, 1-(indole-3)-4-phenyl-3-hydroxy-2-butanone, 4-hydroxyphenylacetoaldehyde, 1,4-di(4-hydroxyphenyl)butane, 1,4-di(4-hydroxyphenyl)-1-butene, 1,4-di(4-hydroxyphenyl)-2-butene, 1,4-di(4-hydroxyphenyl)-2-butanol, 1,4-di(4-hydroxyphenyl)-2-butanone, 1,4-di(4-hydroxyphenyl)-2,3-butanediol, 1,4-di(4-hydroxyphenyl)-3-hydroxy-2-butanone, 1-(4-hydroxyphenyl)-4-(indole-3-)butane, 1-(4-hydroxyphenyl)-4-(indole-3)-1-butene, 1-di(4-hydroxyphenyl)-4-(indole-3)-2-butene, 1-(4-hydroxyphenyl)-4-(indole-3)-2-butanol, 1-(4-hydroxyphenyl)-4-(indole-3)-2-butanone, 1-(4-hydroxyphenyl)-4-(indole-3)-2,3-butanediol, 1-(4-hydroxyphenyl-4-(indole-3)-3-hydroxy-2-butanone, indole-3-acetoaldehyde, 1,4-di(indole-3-)butane, 1,4-di(indole-3)-1-butene, 1,4-di(indole-3)-2-butene, 1,4-di(indole-3)-2-butanol, 1,4-di(indole-3)-2-butanone, 1,4-di(indole-3)-2,3-butanediol, 1,4-di(indole-3)-3-hydroxy-2-butanone, succinate semialdehyde, hexane-1,8-dicarboxylic acid, 3-hexene-1,8-dicarboxylic acid, 3-hydroxy-hexane-1,8-dicarboxylic acid, 3-hexanone-1,8-dicarboxylic acid, 3,4-hexanediol-1,8-dicarboxylic acid, 4-hydroxy-3-hexanone-1,8-dicarboxylic acid, fucoidan, iodine, chlorophyll, carotenoid, calcium, magnesium, iron, sodium, potassium, phosphate, lactic acid, acetic acid, formic acid, or isoprenoids and terpenes.

Biofuel Plant and Process of Producing Biofuel

In one aspect, provided herein is a fuel plant that includes a hydrolysis unit configured to hydrolyze a biomass material comprising a high molecular weight carbohydrate, and a fermentor configured to house a medium and one or more species of microorganisms. In one embodiment the microorganism is Clostridium phytofermentans. In another embodiment, the microorganism is Clostridium sp. Q.D. In another embodiment, the microorganism is Clostridium phytofermentans Q.12 In another embodiment, the microorganism is Clostridium phytofermentans Q.13.

In another aspect, provided herein are methods of making a fuel or chemical end-product that includes combining a microorganism (such as Clostridium phytofermentans, Clostridium sp. Q.D, Clostridium phytofermentans Q.12, Clostridium phytofermentans Q.13 or a similar C5/C6 Clostridium species) and a lignocellulosic material (and/or other biomass material) in a medium, and fermenting the lignocellulosic material under conditions and for a time sufficient to produce a fermentation end-product, (e.g., ethanol, propanol, methane or hydrogen). In one embodiment the fermentation end product is a chemical such as 1,4 diacid (succinic, fumaric and malic), 2,5 furan dicarboxylic acid, 3 hydroxy propionic acid, aspartic acid, glucaric acid, glutamic acid, itaconic acid, levulinic acid, 3-hydroxybutyrolactone, glycerol, sorbitol, xylitol/arabinitol, butanediol, butanol and isopentenyl diphosphate, and isoprenoids or terpenes.

In some embodiments, a process is provided for producing a fermentation end-product from biomass using acid hydrolysis pretreatment. In some embodiments, a process is provided for producing a fermentation end-product from biomass using enzymatic hydrolysis pretreatment. In another embodiment a process is provided for producing a fermentation end-product from biomass using biomass that has not been enzymatically pretreated. In another embodiment a process is provided for producing a fermentation end-product from biomass using biomass that has not been chemically or enzymatically pretreated, but is optionally steam treated.

In another aspect, provided herein are end-products made by any of the processes described herein. Those skilled in the art will appreciate that a number of genetic modifications can be made to the methods exemplified herein. For example, a variety of promoters can be utilized to drive expression of the heterologous genes in a recombinant microorganism (such as Clostridium phytofermentans, Clostridium sp. Q.D, Clostridium phytofermentans Q.12 or Clostridium phytofermentans Q.13). The skilled artisan, having the benefit of the instant disclosure, will be able to readily choose and utilize any one of the various promoters available for this purpose. Similarly, skilled artisans, as a matter of routine preference, can utilize a higher copy number plasmid. In another embodiment, constructs can be prepared for chromosomal integration of the desired genes. Chromosomal integration of foreign genes can offer several advantages over plasmid-based constructions, the latter having certain limitations for commercial processes. Ethanologenic genes have been integrated chromosomally in E. coli B; see Ohta et al. (1991) Appl. Environ. Microbiol. 57:893-900. In general, this is accomplished by purification of a DNA fragment containing (1) the desired genes upstream from an antibiotic resistance gene and (2) a fragment of homologous DNA from the target organism. This DNA can be ligated to form circles without replicons and used for transformation. Thus, the gene of interest can be introduced in a heterologous host such as E. coli, and short, random fragments can be isolated and ligated in Clostridium phytofermentans, Clostridium sp. Q.D, Clostridium phytofermentans Q.12, Clostridium phytofermentans Q.13, or genetically-modified mutants thereof, to promote homologous recombination.

Large Scale Fermentation End-Product Production from Biomass

In one aspect a fermentation end-product (e.g., ethanol) from biomass is produced on a large scale utilizing a microorganism, such as C. phytofermentans, Clostridium sp. Q.D, Clostridium phytofermentans Q.12 or Clostridium phytofermentans Q.13. In one embodiment, one first hydrolyzes a biomass material that includes high molecular weight carbohydrates to lower molecular weight carbohydrates, and then ferments the lower molecular weight carbohydrates utilizing microorganism to produce ethanol. In another embodiment, one ferments the biomass material itself without chemical and/or enzymatic pretreatment. In the first method, hydrolysis can be accomplished using acids, e.g., Bronsted acids (e.g., sulfuric or hydrochloric acid), bases, e.g., sodium hydroxide, hydrothermal processes, steam explosion, ammonia fiber explosion processes (“AFEX”), lime processes, enzymes, or combination of these. Hydrogen, and other products of the fermentation can be captured and purified if desired, or disposed of, e.g., by burning. For example, the hydrogen gas can be flared, or used as an energy source in the process, e.g., to drive a steam boiler, e.g., by burning. Hydrolysis and/or steam treatment of the biomass can, e.g., increase porosity and/or surface area of the biomass, often leaving the cellulosic materials more exposed to the microbial cells, which can increase fermentation rate and yield. Removal of lignin can, e.g., provide a combustible fuel for driving a boiler, and can also, e.g., increase porosity and/or surface area of the biomass, often increasing fermentation rate and yield. In some embodiments, the initial concentration of the carbohydrates in the medium is greater than 20 mM, e.g., greater than 30 mM, 50 mM, 75 mM, 100 mM, 150 mM, 200 mM, or even greater than 500 mM.

In one aspect, the invention features a fuel plant that includes a hydrolysis unit configured to hydrolyze a biomass material that includes a high molecular weight carbohydrate; a fermentor configured to house a medium with a C5/C6 hydrolyzing microorganism (e.g., Clostridium phytofermentans, Clostridium sp. Q.D, Clostridium phytofermentans Q.12 or Clostridium phytofermentans Q.13); and one or more product recovery system(s) to isolate a fermentation end-product or end-products and associated by-products and co-products.

In another aspect, the invention features methods of making a fermentation end-product or end-products that include combining a C5/C6 hydrolyzing microorganism (e.g., Clostridium phytofermentans, Clostridium sp. Q.D, Clostridium phytofermentans Q.12 or Clostridium phytofermentans Q.13) and a carbonaceous biomass in a medium, and fermenting the biomass material under conditions and for a time sufficient to produce a fermentation end-products (e.g. ethanol, propanol, hydrogen, lignin, terpenoids, and the like). In one embodiment the fermentation end-product is a biofuel or chemical product.

In another aspect, the invention features one or more end-products made by any of the processes described herein. In one embodiment one or more fermentation end-products can be produced from biomass on a large scale utilizing a C5/C6 hydrolyzing microorganism (e.g., Clostridium phytofermentans, Clostridium sp. Q.D, Clostridium phytofermentans Q.12 or Clostridium phytofermentans Q.13). In one embodiment depending on the type of biomass and its physical manifestation, the process can comprise a milling of the carbonaceous material, via wet or dry milling, to reduce the material in size and increase the surface to volume ratio (physical modification).

In some embodiments, the treatment includes treatment of a biomass with acid. In some embodiments, the acid is dilute. In some embodiments, the acid treatment is carried out at elevated temperatures of between about 85 and 140° C. In some embodiments, the method further comprises the recovery of the acid treated biomass solids, for example by use of a sieve. In some embodiments, the sieve comprises openings of approximately 150-250 microns in diameter. In some embodiments, the method further comprises washing the acid treated biomass with water or other solvents. In some embodiments, the method further comprises neutralizing the acid with alkali. In some embodiments, the method further comprises drying the acid treated biomass. In some embodiments, the drying step is carried out at elevated temperatures between about 15-45° C. In some embodiments, the liquid portion of the separated material is further treated to remove toxic materials. In some embodiments, the liquid portion is separated from the solid and then fermented separately. In some embodiments, a slurry of solids and liquids are formed from acid treatment and then fermented together.

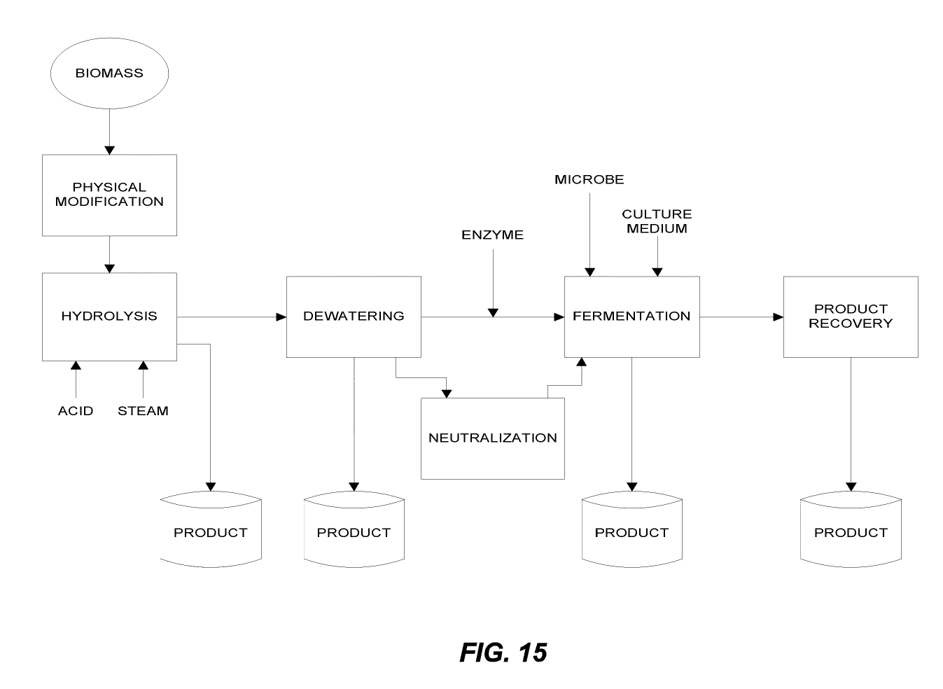

FIG. 15 illustrates an example of a method for producing chemical products from biomass by first treating biomass with an acid at elevated temperature and pressure in a hydrolysis unit. The biomass can first be heated by addition of hot water or steam. The biomass can be acidified by bubbling gaseous sulfur dioxide through the biomass that is suspended in water, or by adding a strong acid, e.g., sulfuric, hydrochloric, or nitric acid with or without preheating/presteaming/water addition. During the acidification, the pH is maintained at a low level, e.g., below about 5. The temperature and pressure can be elevated after acid addition. In addition to the acid already in the acidification unit, optionally, a metal salt such as ferrous sulfate, ferric sulfate, ferric chloride, aluminum sulfate, aluminum chloride, magnesium sulfate, or mixtures of these can be added to aid in the hydrolysis of the biomass. The acid-impregnated biomass is fed into the hydrolysis section of the pretreatment unit. Steam is injected into the hydrolysis portion of the pretreatment unit to directly contact and heat the biomass to the desired temperature. The temperature of the biomass after steam addition is, e.g., between about 130° C. and 220° C. The hydrolysate is then discharged into the flash tank portion of the pretreatment unit, and is held in the tank for a period of time to further hydrolyze the biomass, e.g., into oligosaccharides and monomeric sugars. Steam explosion can also be used to further break down biomass. Alternatively, the biomass can be subject to discharge through a pressure lock for any high-pressure pretreatment process. Hydrolysate is then discharged from the pretreatment reactor, with or without the addition of water, e.g., at solids concentrations between about 15% and 60%.

After pretreatment, the biomass can be dewatered and/or washed with a quantity of water, e.g. by squeezing or by centrifugation, or by filtration using, e.g. a countercurrent extractor, wash press, filter press, pressure filter, a screw conveyor extractor, or a vacuum belt extractor to remove acidified fluid. The acidified fluid, with or without further treatment, e.g. addition of alkali (e.g. lime) and or ammonia (e.g. ammonium phosphate), can be re-used, e.g., in the acidification portion of the pretreatment unit, or added to the fermentation, or collected for other use/treatment. Products can be derived from treatment of the acidified fluid, e.g., gypsum or ammonium phosphate. Enzymes or a mixture of enzymes can be added during pretreatment to assist, e.g. endoglucanases, exoglucanases, cellobiohydrolases (CBH), beta-glucosidases, glycoside hydrolases, glycosyltransferases, lyases, and esterases active against components of cellulose, hemicelluloses, pectin, and starch, in the hydrolysis of high molecular weight components.

In one embodiment the fermentor is fed with hydrolyzed biomass; any liquid fraction from biomass pretreatment; an active seed culture of Clostridium phytofermentans, Clostridium sp. Q.D, Clostridium phytofermentans Q.12, Clostridium phytofermentans Q.13, or mutagenized or genetically-modified cells thereof, if desired a co-fermenting microbe, e.g., yeast or E. coli; and, as needed, nutrients to promote growth of Clostridium phytofermentans or other microbes. In another embodiment the pretreated biomass or liquid fraction can be split into multiple fermentors, each containing a different strain of Clostridium phytofermentans, Clostridium sp. Q.D, Clostridium phytofermentans Q.12, Clostridium phytofermentans Q.13 or the like and/or other microbes; with each fermentor operating under specific physical conditions. Fermentation is allowed to proceed for a period of time, e.g., between about 15 and 150 hours, while maintaining a temperature of, e.g., between about 25° C. and 50° C. Gas produced during the fermentation is swept from fermentor and is discharged, collected, or flared with or without additional processing, e.g. hydrogen gas can be collected and used as a power source or purified as a co-product.

After fermentation, the contents of the fermentor are transferred to product recovery. Products are extracted, e.g., ethanol is recovered through distilled and rectification. Methods and compositions described herein can include extracting or separating fermentation end-products, such as ethanol, from biomass. Depending on the product formed, different methods and processes of recovery can be provided.

In one embodiment, a method for extraction of lactic acid from a fermentation broth uses freezing and thawing of the broth followed by centrifugation, filtration, and evaporation. (Omar, et al. 2009 African J. Biotech. 8:5807-5813) Other methods that can be utilized are membrane filtration, resin adsorption, and crystallization. (See, e.g., Huh, et al. 2006 Process Biochemistry).

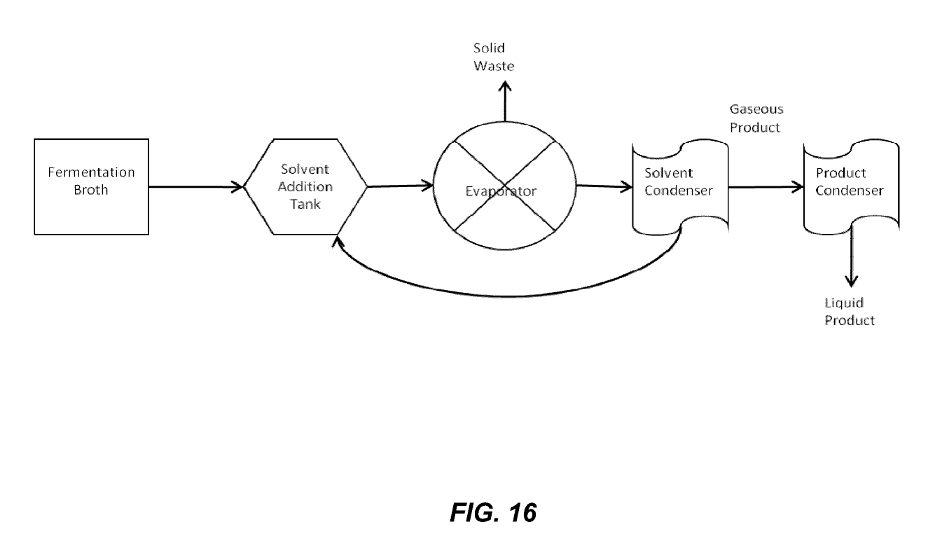

In another embodiment for solvent extraction of a variety of organic acids (such as ethyl lactate, ethyl acetate, formic, butyric, lactic, acetic, succinic), the process can take advantage of preferential partitioning of the product into one phase or the other. In some cases the product might be carried in the aqueous phase rather than the solvent phase. In other embodiments, the pH is manipulated to produce more or less acid from the salt synthesized from the organism. The acid phase is then extracted by vaporization, distillation, or other methods. See FIG. 16.

In one embodiment, a product for production of a chemical product comprises: a carbonaceous biomass, an organism that is capable of direct hydrolysis and fermentation of the biomass to the product, wherein the product is, for example 1, 4 diacid (succinic, fumaric and malic), 2,5 furan dicarboxylic acid, 3-hydroxy propionic acid, aspartic acid, glucaric acid, glutamic acid, itaconic acid, levulinic acid, 3-hydroxybutyrolactone, glycerol, sorbitol, xylitol/arabinitol, butanediol, butanol, isoprenoids, terpenes, or the like.

In another embodiment, a product for production of a chemical product comprises: a carbonaceous biomass, an organism that is capable of direct hydrolysis and fermentation of the biomass, wherein said organism is modified to provide enhanced production of a chemical product such as, but not limited to 1,4 diacid (succinic, fumaric and malic), 2,5 furan dicarboxylic acid, 3-hydroxy propionic acid, aspartic acid, glucaric acid, glutamic acid, itaconic acid, levulinic acid, 3-hydroxybutyrolactone, glycerol, sorbitol, xylitol/arabinitol, butanediol, isopentenyl diphosphate, butanol, and isoprenoids or terpenes.

In yet a further embodiment, a product for production of fermentive end-products comprises: (a) a fermentation vessel comprising a carbonaceous biomass; (b) and a modified organism that is capable of direct hydrolysis and fermentation of the biomass; wherein the fermentation vessel is adapted to provide suitable conditions for fermentation of one or more carbohydrates into fermentive end-products.

In another embodiment, a product for production of a chemical product comprises: a carbonaceous biomass, an organism that is capable of direct hydrolysis and fermentation of the biomass, wherein said organism is modified to provide enhanced production of a chemical product including, but not limited to 1,4 diacid (succinic, fumaric and malic), 2,5 furan dicarboxylic acid, 3-hydroxy propionic acid, aspartic acid, aspartate glucaric acid, glutamic acid, malic acid, malate, glutamate, itaconic acid, levulinic acid, 3-hydroxybutyrolactone, glycerol, sorbitol, xylitol/arabinitol, butanediol, butanol, isoprenoids, terpenes, and isoprenoids.

Chemical Production from Biomass without Pretreatment

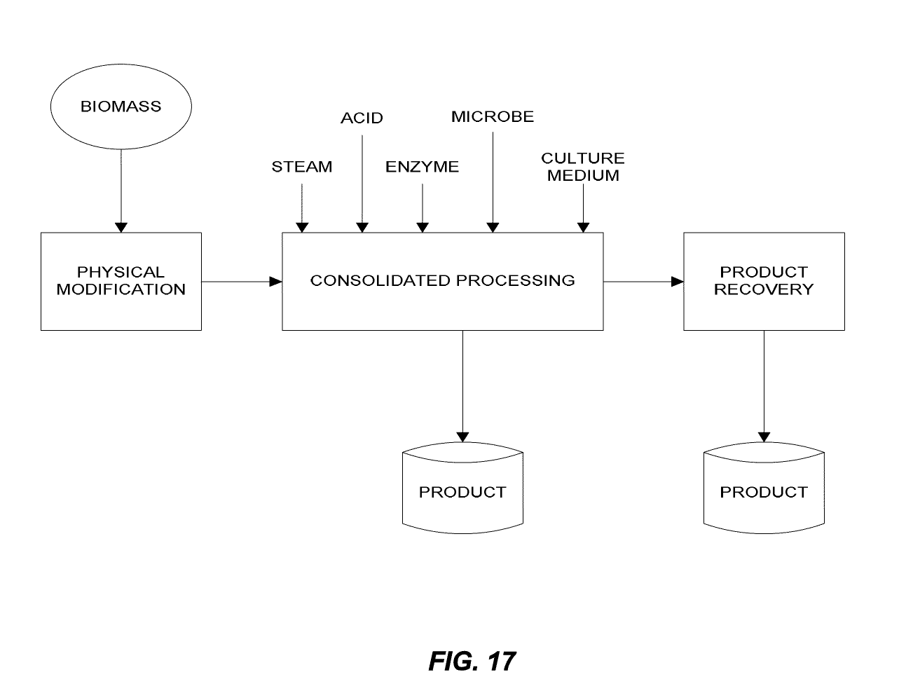

FIG. 17 depicts a method for producing chemicals from biomass by charging biomass to a fermentation vessel. The biomass can be allowed to soak for a period of time, with or without addition of heat, water, enzymes, or acid/alkali. The pressure in the processing vessel can be maintained at or above atmospheric pressure. Acid or alkali can be added at the end of the pretreatment period for neutralization. At the end of the pretreatment period, or at the same time as pretreatment begins, an active seed culture of a C5/C6 hydrolyzing microorganism (e.g., Clostridium phytofermentans, Clostridium sp. Q.D, Clostridium phytofermentans Q.12, or Clostridium phytofermentans Q.13) and, if desired, a co-fermenting microbe, e.g., yeast or E. coli, and, if required, nutrients to promote growth of a C5/C6 hydrolyzing microorganism (e.g., Clostridium phytofermentans, Clostridium sp. Q.D, Clostridium phytofermentans Q.12, Clostridium phytofermentans Q.13, or mutagenize or genetically-modified cells thereof are added. Fermentation is allowed to proceed as described above. After fermentation, the contents of the fermentor are transferred to product recovery as described above. Any combination of the chemical production methods and/or features can be utilized to make a hybrid production method. In any of the methods described herein, products can be removed, added, or combined at any step. A C5/C6 hydrolyzing microorganism (e.g., Clostridium phytofermentans, Clostridium sp. Q.D, Clostridium phytofermentans Q.12, or Clostridium phytofermentans Q.13) can be used alone or synergistically in combination with one or more other microbes (e.g. yeasts, fungi, or other bacteria). In some embodiments different methods can be used within a single plant to produce different end-products.

In another aspect, the invention features a fuel plant that includes a hydrolysis unit configured to hydrolyze a biomass material that includes a high molecular weight carbohydrate, a fermentor configured to house a medium and contains a C5/C6 hydrolyzing microorganism (e.g., Clostridium phytofermentans, Clostridium sp. Q.D, Clostridium phytofermentans Q.12, Clostridium phytofermentans Q.13, or mutagenized or genetically-modified cells thereof).

In another aspect, the invention features a chemical production plant that includes a hydrolysis unit configured to hydrolyze a biomass material that includes a high molecular weight carbohydrate, a fermentor configured to house a medium and contains a C5/C6 hydrolyzing microorganism (e.g., Clostridium phytofermentans, Clostridium sp. Q.D, Clostridium phytofermentans Q.12, Clostridium phytofermentans Q.13, or mutagenized or genetically-modified cells thereof).

In another aspect, the invention features methods of making a chemical(s) or fuel(s) that include combining a C5/C6 hydrolyzing microorganism (e.g., Clostridium phytofermentans, Clostridium sp. Q.D, Clostridium phytofermentans Q.12, or Clostridium phytofermentans Q.13), and a lignocellulosic material (and/or other biomass material) in a medium, and fermenting the lignocellulosic material under conditions and for a time sufficient to produce a a chemical(s) or fuel(s), e.g., ethanol, propanol and/or hydrogen or another chemical compound.

In some embodiments, the present invention provides a process for producing ethanol and hydrogen from biomass using acid hydrolysis pretreatment. In some embodiments, the present invention provides a process for producing ethanol and hydrogen from biomass using enzymatic hydrolysis pretreatment. Other embodiments provide a process for producing ethanol and hydrogen from biomass using biomass that has not been enzymatically pretreated. Still other embodiments disclose a process for producing ethanol and hydrogen from biomass using biomass that has not been chemically or enzymatically pretreated, but is optionally steam treated.

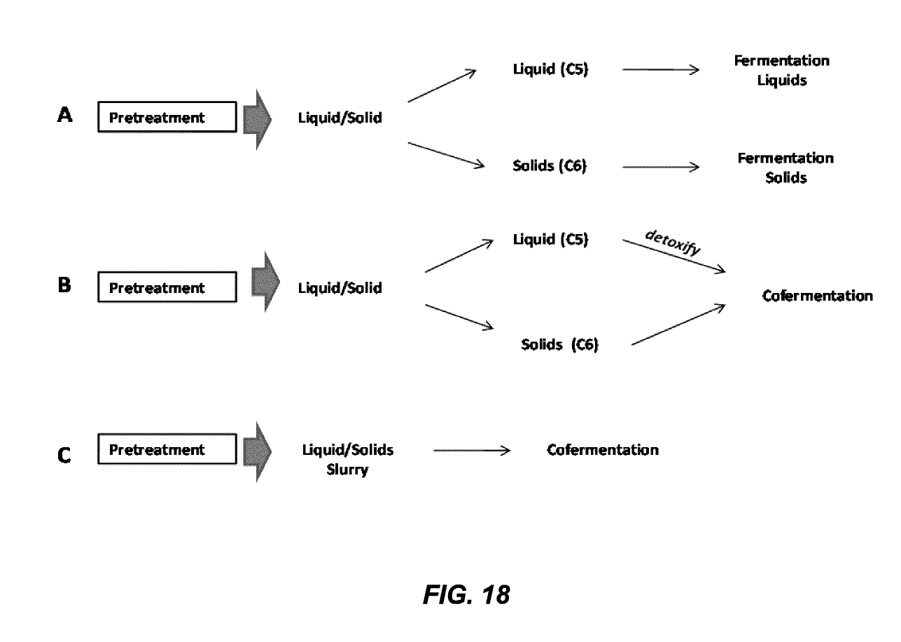

FIG. 18 discloses pretreatments that produce hexose or pentose saccharides or oligomers that are then unprocessed or processed further and either, fermented separately or together. FIG. 18A depicts a process (e.g., acid pretreatment) that produces a solids phase and a liquid phase which are then fermented separately. FIG. 18B depicts a similar pretreatment that produces a solids phase and liquids phase. The liquids phase is separated from the solids and elements that are toxic to the fermenting microorganism are removed prior to fermentation. At initiation of fermentation, the two phases are recombined and cofermented together. This is a more cost-effective process than fermenting the phases separately. The third process (FIG. 18C) is the least costly. The pretreatment results in a slurry of liquids or solids that are then cofermented. There is little loss of saccharides component and minimal equipment required.

Modification to Enhance Enzyme Activity

In one embodiment one or more modifications hydrolysis and/or fermentation conditions can be implemented to enhance end-product production. Examples of such modifications include genetic modification to enhance enzyme activity in a microorganism that already comprises genes for encoding one or more target enzymes, introducing one or more heterogeneous nucleic acid molecules into a host microorganism to express and enhance activity of an enzyme not otherwise expressed in the host, modifying physical and chemical conditions to enhance enzyme function (e.g., modifying and/or maintaining a certain temperature, pH, nutrient concentration, temporal), or a combination of one or more such modifications.

Genetic Modification

In one embodiment, a microorganism can be genetically modified to enhance enzyme activity of one or more enzymes, including but not limited to hydrolytic enzymes (such as cellulase(s), hemicellulase(s), or pectinases etc.). In another embodiment, an enzyme can be selected from the annotated genome of C. phytofermentans, another bacterial species, such as B. subtilis, E. coli, various Clostridium species, or yeasts such as S. cerevisiae for utilization in products and processes described herein. Examples include enzymes such as L-butanediol dehydrogenase, acetoin reductase, 3-hydroxyacyl-CoA dehydrogenase, cis-aconitate decarboxylase or the like, to create pathways for new products from biomass.

Examples of such modifications include modifying endogenous nucleic acid regulatory elements to increase expression of one or more enzymes (e.g., operably linking a gene encoding a target enzyme to a strong promoter), introducing into a microorganism additional copies of endogenous nucleic acid molecules to provide enhanced activity of an enzyme by increasing its production, and operably linking genes encoding one or more enzymes to an inducible promoter or a combination thereof.

In another embodiment a microorganism can be modified to enhance an activity of one or more cellulases, or enzymes associated with cellulose processing. The classification of cellulases is usually based on grouping enzymes together that forms a family with similar or identical activity, but not necessary the same substrate specificity. One of these classifications is the CAZy system (CAZy stands for Carbohydrate-Active enzymes), for example, where there are 115 different Glycoside Hydrolases (GH) listed, named GH1 to GH155. Each of the different protein families usually has a corresponding enzyme activity. This database includes both cellulose and hemicellulase active enzymes. Furthermore, the entire annotated genome of C. phytofermentans is available on the worldwideweb at www.ncbi.nlm.nih.gov/sites/entrez.

Several examples of cellulase enzymes whose function can be enhanced for expression endogenously or for expression heterologously in a microorganism include one or more of the genes disclosed in Table 2.

TABLE 2

| Cellulase Protein ID | Description (on www.ncbi.nlm.nih.gov/sites/entrez) |
|---|---|
| ABX43556 | Cellulase [Clostridium phytofermentans ISDg] |
| | gi\|160429993\|gb\|ABX43556.1\|[160429993] |
| | Cphy_3302 |
| ABX42426 | Cellulase [Clostridium phytofermentans ISDg] |
| | gi\|160428863\|gb\|ABX42426.1\|[160428863] |
| | Cphy_2058 |
| ABX41541 | Cellulase [Clostridium phytofermentans ISDg] |
| | gi\|160427978\|gb\|ABX41541.1\|[160427978] |
| | Cphy_1163 |
| ABX43720 | Cellulose 1,4-beta-cellobiosidase [Clostridium |
| | phytofermentans ISDg] |
| | gi\|160430157\|gb\|ABX43720.1\|[160430157] |
| | Cphy_3367 |
| ABX41478 | Cellulase M Cphy_1100 |
| ABX41884 | Endo-1,4-beta-xylanase Cphy_1510 |
| ABX43721 | Cellulase 1,4-beta-cellobiosidase Cphy_3368 |
| ABX42494 | Mannan endo-1,4-beta-mannosidase, Cellulase |
| | 1,4-beta-cellobiosidase Cphy_2128 |

Several examples of cThe Glycosyl hydrolase family 9 (GH9): O-Glycosyl hydrolases are a widespread group of enzymes that hydrolyse the glycosidic bond between two or more carbohydrates, or between a carbohydrate and a non-carbohydrate moiety. A classification system for glycosyl hydrolases, based on sequence similarity, has led to the definition of 85 different families PUBMED:7624375, PUBMED:8535779, PUBMED:. This classification is available on the CAZy (CArbohydrate-Active EnZymes) web site PUBMED. Because the fold of proteins is better conserved than their sequences, some of the families can be grouped in ‘clans’. The Glycoside hydrolase family 9 comprises enzymes with several known activities, such as endoglucanase and cellobiohydrolase. In C. phytofermentans, a GH9 cellulase is ABX43720 (Table 2).

Cellulase enzyme activity can be enhanced in a microorganism. In one embodiment a cellulase disclosed in Table 2 is enhanced in a microorganism.

In one embodiment a hydrolytic enzyme is selected from the annotated genome of C. phytofermentans for utilization in a product or process disclosed herein. In one embodiment the hydrolytic enzyme is an endoglucanase, chitinase, cellobiohydrolase or endo-processive cellulases (either on reducing or non-reducing end).

In one embodiment a microorganism, such as C. phytofermentans, can be modified to enhance production of one or more hydrolases. In another embodiment one or more enzymes can be heterologous expressed in a host (e.g., a bacteria or yeast). For heterologous expression bacteria or yeast can be modified through recombinant technology. (e.g., Brat et al. Appl. Env. Microbio. 2009; 75(8):2304-2311, disclosing expression of xylose isomerase in S. cerevisiae and which is herein incorporated by reference in its entirety).

In another embodiment other modifications can be made to enhance end-product (e.g., ethanol) production in a recombinant microorganism. For example, the host microorganism can further comprise an additional heterologous DNA segment, the expression product of which is a protein involved in the transport of mono- and/or oligosaccharides into the recombinant host. Likewise, additional genes from the glycolytic pathway can be incorporated into the host. In such ways, an enhanced rate of ethanol production can be achieved.

A variety of promoters (e.g., constitutive promoters, inducible promoters) can be used to drive expression of the heterologous genes in a recombinant host microorganism.

Promoter elements can be selected and mobilized in a vector (e.g., pIMPCphy). For example, a transcription regulatory sequence is operably linked to gene(s) of interest (e.g., in a expression construct). The promoter can be any array of DNA sequences that interact specifically with cellular transcription factors to regulate transcription of the downstream gene. The selection of a particular promoter depends on what cell type is to be used to express the protein of interest. In one embodiment a transcription regulatory sequences can be derived from the host microorganism. In various embodiments, constitutive or inducible promoters are selected for use in a host cell. Depending on the host cell, there are potentially hundreds of constitutive and inducible promoters which are known and that can be engineered to function in the host cell.

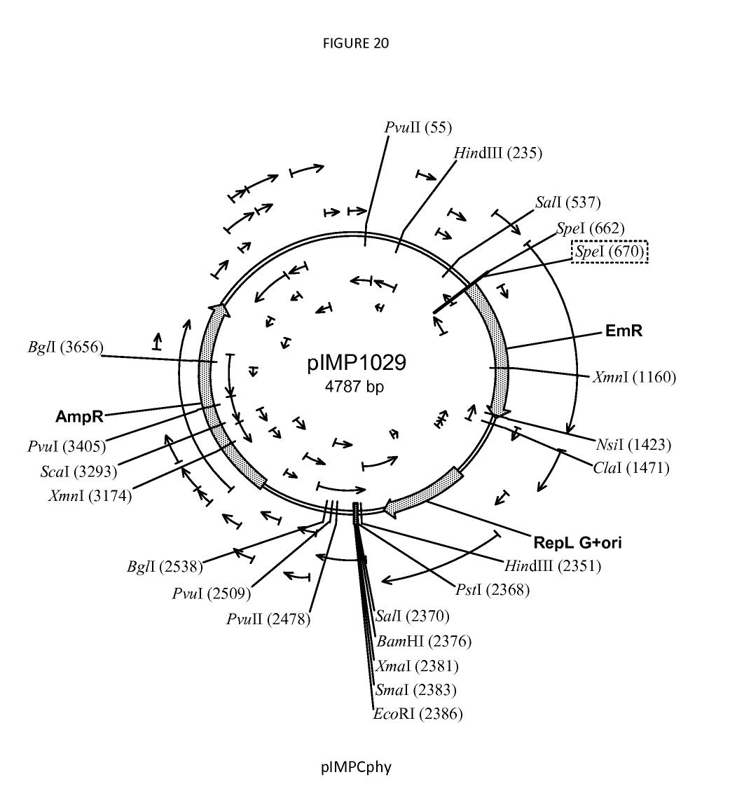

A map of the plasmid pIMPCphy is shown in FIG. 20, and the DNA sequence of this plasmid is provided as SEQ ID NO:1.

SEQ ID NO: 1:
gcgcccaatacgcaaaccgcctctccccgcgcgttggccgattcattaa
tgcagctggcacgacaggtttcccgactggaaagcgggcagtgagcgca
acgcaattaatgtgagttagctcactcattaggcaccccaggctttaca
ctttatgcttccggctcgtatgttgtgtggaattgtgagcggataacaa
tttcacacaggaaacagctatgaccatgattacgccaaagctttggcta
acacacacgccattccaaccaatagttttctcggcataaagccatgctc
tgacgcttaaatgcactaatgccttaaaaaaacattaaagtctaacaca
ctagacttatttacttcgtaattaagtcgttaaaccgtgtgctacgacc
aaaagtataaaacctttaagaactttatttttcttgtaaaaaaagaaac
tagataaatctctcatatcttttattcaataatcgcatcagattgcagt
ataaatttaacgatcactcatcatgttcatatttatcagagctccttat
attttatttcgatttatttgttatttatttaacatttttctattgacct
catcttttctatgtgttattcttttgttaattgtttacaaataatctac
gatacatagaaggaggaaaaactagtatactagtatgaacgagaaaaat
ataaaacacagtcaaaactttattacttcaaaacataatatagataaaa
taatgacaaatataagattaaatgaacatgataatatctttgaaatcgg
ctcaggaaaagggcattttacccttgaattagtacagaggtgtaatttc
gtaactgccattgaaatagaccataaattatgcaaaactacagaaaata
aacttgttgatcacgataatttccaagttttaaacaaggatatattgca
gtttaaatttcctaaaaaccaatcctataaaatatttggtaatatacct
tataacataagtacggatataatacgcaaaattgtttttgatagtatag
ctgatgagatttatttaatcgtggaatacgggtttgctaaaagattatt
aaatacaaaacgctcattggcattatttttaatggcagaagttgatatt
tctatattaagtatggttccaagagaatattttcatcctaaacctaaag
tgaatagctcacttatcagattaaatagaaaaaaatcaagaatatcaca
caaagataaacagaagtataattatttcgttatgaaatgggttaacaaa
gaatacaagaaaatatttacaaaaaatcaatttaacaattccttaaaac
atgcaggaattgacgatttaaacaatattagcttgaacaattcttatct
cttttcaatagctataaattatttaataagtaagttaagggatgcataa
actgcatcccttaacttgtttttcgtgtacctattttttgtgaatcgat
ccggccagcctcgcagagcaggattcccgttgagcaccgccaggtgcga
ataagggacagtgaagaaggaacacccgctcgcgggtgggcctacttca
cctatcctgcccggatcgattatgtcttttgcgcattcacttcttttct
atataaatatgagcgaagcgaataagcgtcggaaaagcagcaaaaagtt
tcctttttgctgttggagcatgggggttcagggggtgcagtatctgacg
tcaatgccgagcgaaagcgagccgaagggtagcatttacgttagataac
cccctgatatgctccgacgctttatatagaaaagaagattcaactaggt
aaaatcttaatataggttgagatgataaggtttataaggaatttgtttg
ttctaatttttcactcattttgttctaatttcttttaacaaatgttctt
ttttttttagaacagttatgatatagttagaatagtttaaaataaggag
tgagaaaaagatgaaagaaagatatggaacagtctataaaggctctcag
aggctcatagacgaagaaagtggagaagtcatagaggtagacaagttat
accgtaaacaaacgtctggtaacttcgtaaaggcatatatagtgcaatt
aataagtatgttagatatgattggcggaaaaaaacttaaaatcgttaac
tatatcctagataatgtccacttaagtaacaatacaatgatagctacaa
caagagaaatagcaaaagctacaggaacaagtctacaaacagtaataac
aacacttaaaatcttagaagaaggaaatattataaaaagaaaaactgga
gtattaatgttaaaccctgaactactaatgagaggcgacgaccaaaaac
aaaaatacctcttactcgaatttgggaactttgagcaagaggcaaatga
aatagattgacctcccaataacaccacgtagttattgggaggtcaatct
atgaaatgcgattaagcttagcttggctgcaggtcgacggatccccggg
aattcactggccgtcgttttacaacgtcgtgactgggaaaaccctggcg
ttacccaacttaatcgccttgcagcacatccccctttcgccagctggcg
taatagcgaagaggcccgcaccgatcgcccttcccaacagttgcgcagc
ctgaatggcgaatggcgcctgatgcggtattttctccttacgcatctgt
gcggtatttcacaccgcatatggtgcactctcagtacaatctgctctga
tgccgcatagttaagccagccccgacacccgccaacacccgctgacgcg
ccctgacgggcttgtagctcccggcatccgcttacagacaagctgtgac
cgtctccgggagctgcatgtgtcagaggttttcaccgtcatcaccgaaa
cgcgcgagacgaaagggcctcgtgatacgcctatttttataggttaatg
tcatgataataatggtttcttagacgtcaggtggcacttttcggggaaa
tgtgcgcggaacccctatttgtttatttttctaaatacattcaaatatg
tatccgctcatgagacaataaccctgataaatgcttcaataatattgaa
aaaggaagagtatgagtattcaacatttccgtgtcgcccttattccctt
ttttgcggcattttgccttcctgtttttgctcacccagaaacgctggtg
aaagtaaaagatgctgaagatcagttgggtgcacgagtgggttacatcg
aactggatctcaacagcggtaagatccttgagagttttcgccccgaaga
acgttttccaatgatgagcacttttaaagttctgctatgtggcgcggta
ttatcccgtattgacgccgggcaagagcaactcggtcgccgcatacact
attctcagaatgacttggttgagtactcaccagtcacagaaaagcatct
tacggatggcatgacagtaagagaattatgcagtgctgccataaccatg
agtgataacactgcggccaacttacttctgacaacgatcggaggaccga
aggagctaaccgcttttttgcacaacatgggggatcatgtaactcgcct
tgatcgttgggaaccggagctgaatgaagccataccaaacgacgagcgt
gacaccacgatgcctgtagcaatggcaacaacgttgcgcaaactattaa
ctggcgaactacttactctagcttcccggcaacaattaatagactggat
ggaggcggataaagttgcaggaccacttctgcgctcggcccttccggct
ggctggtttattgctgataaatctggagccggtgagcgtgggtctcgcg
gtatcattgcagcactggggccagatggtaagccctcccgtatcgtagt
tatctacacgacggggagtcaggcaactatggatgaacgaaatagacag
atcgctgagataggtgcctcactgattaagcattggtaactgtcagacc
aagtttactcatatatactttagattgatttaaaacttcatttttaatt
taaaaggatctaggtgaagatcctttttgataatctcatgaccaaaatc
ccttaacgtgagttttcgttccactgagcgtcagaccccgtagaaaaga
tcaaaggatcttcttgagatcctttttttctgcgcgtaatctgctgctt
gcaaacaaaaaaaccaccgctaccagcggtggtttgtttgccggatcaa
gagctaccaactctttttccgaaggtaactggcttcagcagagcgcaga
taccaaatactgtccttctagtgtagccgtagttaggccaccacttcaa
gaactctgtagcaccgcctacatacctcgctctgctaatcctgttacca
gtggctgctgccagtggcgataagtcgtgtcttaccgggttggactcaa
gacgatagttaccggataaggcgcagcggtcgggctgaacggggggttc
gtgcacacagcccagcttggagcgaacgacctacaccgaactgagatac
ctacagcgtgagctatgagaaagcgccacgcttcccgaagggagaaagg
cggacaggtatccggtaagcggcagggtcggaacaggagagcgcacgag
ggagcttccagggggaaacgcctggtatctttatagtcctgtcgggttt
cgccacctctgacttgagcgtcgatttttgtgatgctcgtcaggggggc
ggagcctatggaaaaacgccagcaacgcggcctttttacggttcctggc
cttttgctggccttttgctcacatgttctttcctgcgttatcccctgat
tctgtggataaccgtattaccgcctttgagtgagctgataccgctcgcc
gcagccgaacgccgagcgcagcgagtcagtgagcgaggaagcggaaga

The vector pIMPCphy was constructed as a shuttle vector for C. phytofermentans and is further described in U.S. Patent Application Publication US20100086981, which is herein incorporated by reference in its entirety. It has an Ampicillin-resistance cassette and an Origin of Replication (ori) for selection and replication in E. coli. It contains a Gram-positive origin of replication that allows the replication of the plasmid in C. phytofermentans. In order to select for the presence of the plasmid, the pIMPCphy carries an erythromycin resistance gene under the control of the C. phytofermentans promoter of the gene Cphy1029. This plasmid can be transferred to C. phytofermentans by electroporation or by transconjugation with an E. coli strain that has a mobilizing plasmid, for example pRK2030. A plasmid map of pIMPCphy is depicted in FIG. 20. pIMPCphy is an effective replicative vector system for all microbes, including all gram⁺ and gram⁻ bacteria, and fungi (including yeasts).

Promoters typically used in recombinant technology, such as E. coli lac and trp operons, the tac promoter, the bacteriophage pL promoter, bacteriophage T7 and SP6 promoters, beta-actin promoter, insulin promoter, baculoviral polyhedrin and p10 promoter, can be used to initiate transcription.

In one embodiment a constitutive promoter can be used including, but not limited to the int promoter of bacteriophage lamda, the bla promoter of the beta-lactamase gene sequence of pBR322, hydA or thlA in Clostridium, S. coelicolor hrdB, or whiE, the CAT promoter of the chloramphenicol acetyl transferase gene sequence of pPR325, Staphylococcal constitutive promoter blaZ and the like.

In another embodiment an inducible promoter can be used that regulates the expression of downstream gene in a controlled manner, such as under a specific condition of a cell culture. Examples of inducible prokaryotic promoters include, but are not limited to, the major right and left promoters of bacteriophage, the trp, reca, lacZ, AraC and gal promoters of E. coli, the alpha-amylase (Ulmanen Ett at., J. Bacteriol. 162:176-182, 1985, which is herein incorporated by reference in its entirety) and the sigma-28-specific promoters of B. subtilis (Gilman et al., Gene sequence 32:11-20 (1984), which is herein incorporated by reference in its entirety), the promoters of the bacteriophages of Bacillus (Gryczan, In: The Molecular Biology of the Bacilli, Academic Press, Inc., NY (1982), which is herein incorporated by reference in its entirety), Streptomyces promoters (Ward et al., Mol. Gen. Genet. 203:468-478, 1986, which is herein incorporated by reference in its entirety), and the like. Exemplary prokaryotic promoters are reviewed by Glick (J. Ind. Microtiot. 1:277-282, 1987, which is herein incorporated by reference in its entirety); Cenatiempo (Biochimie 68:505-516, 1986, which is herein incorporated by reference in its entirety); and Gottesman (Ann. Rev. Genet. 18:415-442, 1984, which is herein incorporated by reference in its entirety).

A promoter that is constitutively active under certain culture conditions, can be inactive in other conditions. For example, the promoter of the hydA gene from Clostridium acetobutylicum, wherein expression is known to be regulated by the environmental pH. Furthermore, temperature-regulated promoters are also known and can be used. In some embodiments, depending on the desired host cell, a pH-regulated or temperature-regulated promoter can be used with an expression constructs to initiate transcrription. Other pH-regulatable promoters are known, such as P170 functioning in lactic acid bacteria, as disclosed in US Patent Application No. 20020137140, which is herein incorporated by reference in its entirety.

In general, to express the desired gene/nucleotide sequence efficiently, various promoters can be used; e.g., the original promoter of the gene, promoters of antibiotic resistance genes such as for instance kanamycin resistant gene of Tn5, ampicillin resistant gene of pBR322, and promoters of lambda phage and any promoters which can be functional in the host cell. For expression, other regulatory elements, such as for instance a Shine-Dalgarno (SD) sequence (e.g., AGGAGG and so on including natural and synthetic sequences operable in a host cell) and a transcriptional terminator (inverted repeat structure including any natural and synthetic sequence) which are operable in a host cell (into which a coding sequence is introduced to provide a recombinant cell) can be used with the above described promoters.

Examples of promoters that can be used with a product or process disclosed herein include those disclosed in the following patent documents: US20040171824, U.S. Pat. No. 6,410,317, WO 2005/024019, which are herein incorporated by reference in their entirety. Several promoter-operator systems, such as lac, (D. V. Goeddel et al., “Expression in Escherichia coli of Chemically Synthesized Genes for Human Insulin”, Proc. Nat. Acad. Sci. U.S.A., 76:106-110 (1979), which is herein incorporated by reference in its entirety); trp (J. D. Windass et al. “The Construction of a Synthetic Escherichia coli Trp Promoter and Its Use in the Expression of a Synthetic Interferon Gene”, Nucl. Acids. Res., 10:6639-57 (1982), which is herein incorporated by reference in its entirety) and λPL operons (R. Crowl et al., “Versatile Expression Vectors for High-Level Synthesis of Cloned Gene Products in Escherichia coli”, Gene, 38:31-38 (1985), which is herein incorporated by reference in its entirety) in E. coli and have been used for the regulation of gene expression in recombinant cells. The corresponding repressors are the lac repressor, trpR and cI, respectively.

Repressors are protein molecules that bind specifically to particular operators. For example, the lac repressor molecule binds to the operator of the lac promoter-operator system, while the cro repressor binds to the operator of the lambda pR promoter. Other combinations of repressor and operator are known in the art. See, e.g., J. D. Watson et al., Molecular Biology Of The Gene, p. 373 (4th ed. 1987), which is herein incorporated by reference in its entirety. The structure formed by the repressor and operator blocks the productive interaction of the associated promoter with RNA polymerase, thereby preventing transcription. Other molecules, termed inducers, bind to repressors, thereby preventing the repressor from binding to its operator. Thus, the suppression of protein expression by repressor molecules can be reversed by reducing the concentration of repressor (depression) or by neutralizing the repressor with an inducer.

Analogous promoter-operator systems and inducers are known in other microorganisms. In yeast, the GAL10 and GAL1 promoters are repressed by extracellular glucose, and activated by addition of galactose, an inducer. Protein GAL80 is a repressor for the system, and GAL4 is a transcriptional activator. Binding of GAL80 to galactose prevents GAL80 from binding GAL4. Then, GAL4 can bind to an upstream activation sequence (UAS) activating transcription. See Y. Oshima, “Regulatory Circuits For Gene Expression: The Metabolisms Of Galactose And Phosphate” in The Molecular Biology Of The Yeast Sacharomyces, Metabolism And Gene Expression, J. N. Strathern et al. eds. (1982), which are herein incorporated by reference in their entirety.

Transcription under the control of the PHOS promoter is repressed by extracellular inorganic phosphate, and induced to a high level when phosphate is depleted. R. A. Kramer and N. Andersen, “Isolation of Yeast Genes With mRNA Levels Controlled By Phosphate Concentration”, Proc. Nat. Acad. Sci. U.S.A., 77:6451-6545 (1980), which is herein incorporated by reference in its entirety. A number of regulatory genes for PHOS expression have been identified, including some involved in phosphate regulation.

Matα2 is a temperature-regulated promoter system in yeast. A repressor protein, operator and promoter sites have been identified in this system. A. Z. Sledziewski et al., “Construction Of Temperature-Regulated Yeast Promoters Using The Matα2 Repression System”, Bio/Technology, 6:411-16 (1988), which is herein incorporated by reference in its entirety.

Another example of a repressor system in yeast is the CUP1 promoter, which can be induced by Cu⁺2 ions. The CUP1 promoter is regulated by a metallothionine protein. J. A. Gorman et al., “Regulation Of The Yeast Metallothionine Gene”, Gene, 48:13-22 (1986), which is herein incorporated by reference in its entirety.

Similarly, to obtain a desired expression level of one or more cellulase, a higher copy number plasmid can be used. Constructs can be prepared for chromosomal integration of the desired genes. Chromosomal integration of foreign genes can offer several advantages over plasmid-based constructions. Ethanologenic genes have been integrated chromosomally in E. coli B; see Ohta et al. (1991) Appl. Environ. Microbiol. 57:893-900, which is herein incorporated by reference in its entirety. In general, this is accomplished by purification of a DNA fragment containing (1) the desired genes upstream from an antibiotic resistance gene and (2) a fragment of homologous DNA from the target microorganism. This DNA can be ligated to form circles without replicons and used for transformation. Thus, the gene of interest can be introduced in a heterologous host such as E. coli, and short, random fragments can be isolated and operably linked to target genes (e.g., genes encoding cellulase enzymes) to promote homologous recombination.

In another embodiment, organisms are genetically-modified strains of bacteria, including Clostridium sp., including C. phytofermentans. Bacteria comprising altered expression or structure of a gene or genes relative to the original organisms strain, wherein such genetic modifications result in increased efficiency of chemical production. In some embodiments, the genetic modifications are introduced by genetic recombination. In some embodiments, the genetic modifications are introduced by nucleic acid transformation. In further embodiments, the genetic modifications encompass inactivation of one or more genes of Clostridium sp., including C. phytofermentans through any number of genetic methods, including but not limited to single-crossover or double-crossover gene replacement, transposable element insertion, integrational plasmid technology (e.g., using non-replicative or replicative integrative plasmids), targeted gene inactivation using group II intron-based Targetron technology (Chen Y. et al. (2005) Appl Environ Microbial 71:7542-7547), or targeted gene inactivation using ClosTron Group II intron directed mutagenesis (Heap J T et al. (2010) J. Microbiol. Methods 80:49-55. The restriction and modification system of a Clostridium sp. can be modified to increase the efficiency of transformation with unmethylated DNA (Dong H. et al. (2010) PLOS One 5(2): e9038). Interspecific conjugation (for example, with E. coli), can be used to transfer nucleic acid into a Clostridium sp. (Tolonen A C et al. (2009) Molecular Microbiology, 74: 1300-1313). In some strains, genetic modification can comprise inactivation of one or more endogenous nucleic acid sequence(s) and also comprise introduction and activation of heterologous or exogenous nucleic acid sequence(s) and promoters.

In some embodiments, a microorganism can be obtained without the use of recombinant DNA techniques that exhibit desirable properties such as increased productivity, increased yield, or increased titer. For example, mutagenesis, or random mutagenesis can be performed by chemical means or by irradiation of the microorganism. The population of mutagenized microorganisms can then be screened for beneficial mutations that exhibit one or more desirable properties. Screening can be performed by growing the mutagenized microorganisms on substrates that comprise carbon sources that will be used during the generation of end-products by fermentation. Screening can also include measuring the production of end-products during growth of the microorganism, or measuring the digestion or assimilation of the carbon source(s). The isolates so obtained can further be transformed with recombinant polynucleotides or used in combination with any of the methods and compositions provided herein to further enhance biofuel production.

In some embodiments host cells (e.g., microorganisms) can be transformed with one or more polynucleotide encoding one or more enzymes. For example, a single transformed cell can contain exogenous nucleic acids encoding an entire biodegradation pathway. One example of a pathway can include a polynucleotide encoding an exo-β-glucanase, and endo-β-glucanase, and an endoxylanase. Such cells transformed with entire pathways and/or enzymes extracted from them, can saccharify certain components of biomass more rapidly than the naturally-occurring organism. Constructs can contain multiple copies of the same polynucleotide, and/or multiple polynucleotides encoding the same enzyme from different organisms, and/or multiple polynucleotides with mutations in one or more parts of the coding sequences. In some embodiments, the polynucleotides can be similar or identical to the endogenous gene. There can be a percent similarity of 70% (e.g. 70, 75, 80, 85, 90, or 95%) or more in comparing the base pairs of the sequences.

In another embodiment, more effective biomass degradation pathways can be created by transforming host cells with multiple copies of polynucleotides encoding enzymes of the pathway and then combining the cells producing the individual enzymes. This approach allows for the combination of enzymes to more particularly match the biomass of interest by altering the relative ratios of the multiple-transformed strains. In one embodiment two times as many cells expressing the first enzyme of a pathway can be added to a mix where the first step of the reaction pathway is a limiting step of the overall reaction pathway.

In one embodiment biomass-degrading enzymes are made by transforming host cells (e.g., microbial cells such as bacteria, especially Clostridial cells, algae, and fungi) and/or organisms comprising host cells with polynucleotides encoding one or more different biomass degrading enzymes (e.g., cellulolytic enzymes, hemicellulolytic enzymes, xylanases, lignases and cellulases). In some embodiments, a single enzyme can be produced. For example, a cellulase which breaks down pretreated cellulose fragments into cellodextrins or double glucose molecules (cellobiose) or a cellulase which splits cellobiose into glucose, can be produced. In other embodiments, multiple copies of an enzyme can be transformed into an organism to overcome a rate-limiting step of a reaction pathway.

Directed Evolution

Various methods can be used to produce and select mutants that differ from wild-type cells. In some instances, bacterial populations are treated with a mutagenic agent, for example, nitrosoguanidine (N-methyl-N′-nitro-N-nitrosoguanidine) or the like, to increase the mutation frequency above that of spontaneous mutagenesis. This is induced mutagenesis. Techniques for inducing mutagenesis include, but are not limited to, exposure of the bacteria to a mutagenic agent, such as x-rays or chemical mutagenic agents. More sophisticated procedures involve isolating the gene of interest and making a change in the desired location, then reinserting the gene into bacterial cells. This is site-directed mutagenesis.

Directed evolution is usually performed as three steps which can be repeated more than once. First, the gene encoding a protein of interest is mutated and/or recombined at random to create a large library of gene variants. The library is then screened or selected for the presence of mutants or variants that show the desired property. Screens enable the identification and isolation of high-performing mutants by hand; selections automatically eliminate all non functional mutants. Then the variants identified in the selection or screen are replicated, enabling DNA sequencing to determine what mutations occurred. Directed evolution can be carried out in vivo or in vitro. See, e.g., Otten, L. G.; Quax, W. J. (2005). Biomolecular Engineering 22 (1-3): 1-9; Yuan, L., et al. (2005) Microbiol. Mol. Biol. Rev. 69 (3): 373-392.

In other embodiments, a microorganism can be obtained without the use of recombinant DNA techniques that exhibit desirable properties such as increased productivity, increased yield, or increased titer. For example, mutagenesis, or random mutagenesis can be performed by chemical means or by irradiation of the microorganism. The population of mutagenised microorganisms can then be screened for beneficial mutations that exhibit one or more desirable properties. Screening can be performed by growing the mutagenised microorganisms on substrates that comprise carbon sources that will be utilized during the generation of end-products by fermentation. Screening can also include measuring the production of end-products during growth of the microorganism, or measuring the digestion or assimilation of the carbon source(s). The isolates so obtained can further be transformed with recombinant polynucleotides or used in combination with any of the methods and compositions provided herein to further enhance biofuel production.

### EXAMPLES

### Example 1

### Glycerol Production

There are two possible pathways by which C. phytofermentans can produce glycerol as an end product. The synthesis of glycerol begins with glycerol-3-phosphate (FIG. 1.).

C. phytofermentans encodes dihydroxyacetone kinase EC2.7.1.29 (Cphy_{—}1262) and therefore can convert a product of glycolysis, Glycerone phosphate (dihydroxyacetone phosphate) to glycerone. In the alternative, Cphy_{—}1263 can also convert Glycerone phosphate to glycerone. C. phytofermentans does not encode a glycerone dehydrogenase, an enzyme that converts glycerone to glycerol. However, copies of this gene are present in C. perfringens, C. novyi, and C. beijerinckii. A blast search, using the sequence from these species, was performed in the C. phytofermentans genome. The closest hits were Cphy_{—}2463 and Cphy_{—}2650 (E-value smaller than 2e-4). Cphy_{—}2463 and Cphy_{—}2650 are annotated as iron-containing alcohol dehydrogenase, but from the conserved domain analysis, the protein sequences are shown to have the domain of GldA (glycerol dehydrogenase and related enzymes; E-value smaller than 1e-13).

Glycerol can also be produced by the reduction from glycerone phosphate via NAD (P) H-dependent glycerol-3-phosphate dehydrogenase EC 1.1.1.94 (Cphy_{—}2388) or Cphy_{—}3205.

Glycerol can also be formed by the reduction of dihydroxyacetone phosphate (DHAP) to glycerol 3-phosphate by FAD-dependent oxidoreductase using Cphy_{—}0320 [EC 1.1.5.3] or Cphy_{—}3394. Dihydroxyacetone phosphate (DHAP) can be obtained from fructose 1,6-bisphosphate. Glycerol 3-phosphate is then dephosphorylated to glycerol by glycerol 3-phosphatase (Gpp p).

### Example 2

### Xylitol and Arabitol Production

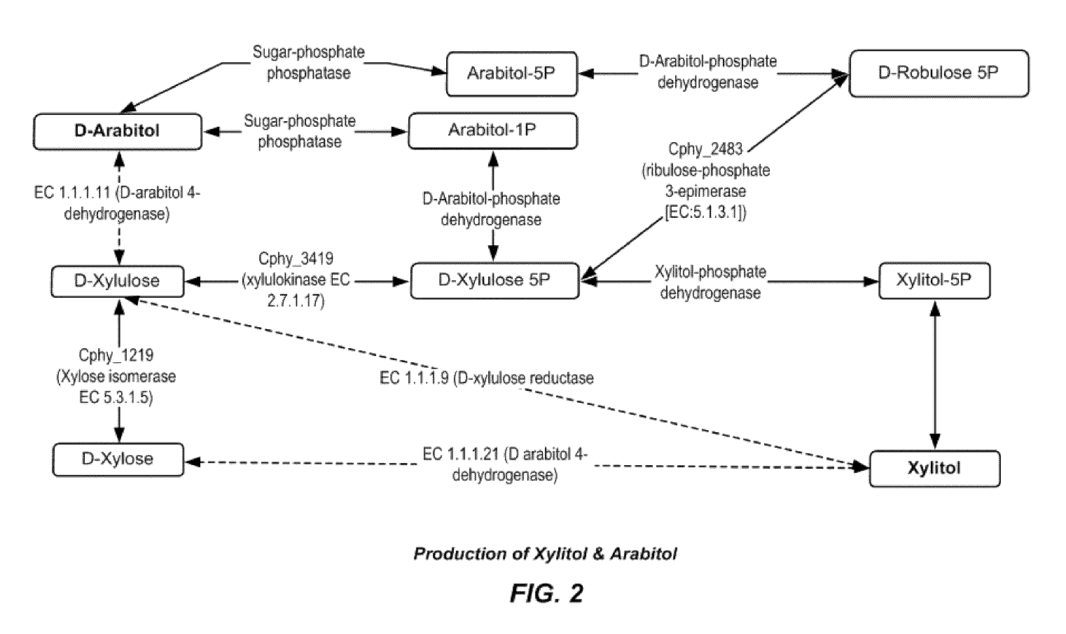

In the conventional pentose and glucuronate interconversions pathway, xylitol and arabitol can be made from D-xylulose by D-xylulose reductase (EC 1.1.1.9) and D-arabitol 4-dehydrogenases (EC1.1.1.11) respectively. This conversion consists of two successive reactions, conversion of D-arabitol to D-xylulose by a membrane-bound D-arabitol dehydrogenase, and conversion of D-xylulose to xylitol by a soluble NAD-dependent xylitol dehydrogenase (FIG. 2).

Polyelainen and Miasnikov (Povelainen, M. and A. N. Miasnikov, 2006 Biotechnol. J. 1:214-219; Povelainen, M. and A. N. Miasnikov, 2007 J. Biotech. 128:24-31) isolated the xylitol-phosphate dehydrogenases and D-arabitol-phosphate dehydrogenases from several gram-positive bacteria and expressed them in Bacillus subtilis. The metabolically engineered strains of B. subtilis were able to convert D-glucose to xylitol with 23% yield, and to D-arabitol with 38% yield. In addition, xylitol can be produced from glucose by means of three sequential steps (glucose->D-arabitol-+D-xylulose->xylitol). The xylitol formation can be carried out without isolation and purification of the intermediates with a yield of approximately 11% xylitol from glucose.

### Example 3

### Sorbitol (D-Glucitol) Production

Gay (Chalumeau, H., A. Delobbe, and P. Gay. 1978 J. Bacteriol. 134(3):920-928) characterized D-sorbitol dehydrogenases (GutB) in Bacillus subtilis, which converts D-fructose to sorbitol. This gene is also found in Clostridium difficile.

Taking the sequences of GutB from B. subtilis and C. difficile to perform a blast search in C. phytofermentans, the closest hit is Cphy_{—}1179 (E-value smaller than 2E-22), which is annotated as alcohol dehydrogenase zinc binding domain.

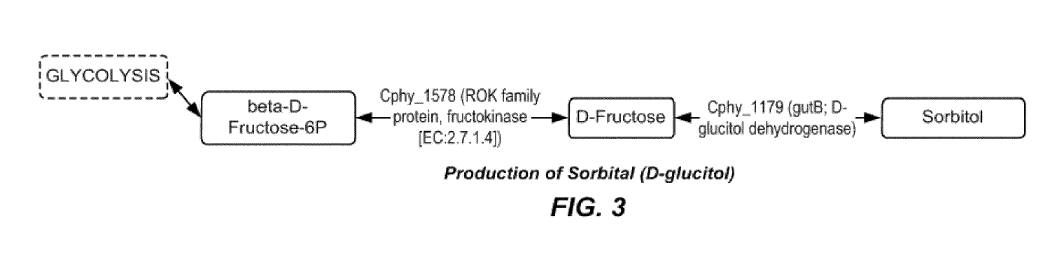

Nissen L. discloses that a recombinant strain of L. casei can be constructed by the integration of a D-sorbitol-6-phosphate dehydrogenase-encoding gene (gutF) in the chromosomal lactose operon (strain BL232). Expression of gutF in this strain generally followed the same regulation as that of the lac genes, that is, it was repressed by glucose and induced by lactose. ¹³C-nuclear magnetic resonance analysis of supernatants of BL232 resting cells demonstrated that, when pre-grown on lactose, cells were able to synthesize sorbitol from glucose. Inactivation of the L-lactate dehydrogenase gene in BL232 led to an increase in sorbitol production, suggesting that the engineered route provided an alternative pathway for NAD regeneration. (Nissen L., et al. FEMS microbiology letters, 2005, vol. 249, n^{o}1, pp. 177-183, which is herein incorporated by reference in its entirety). See FIG. 3.

### Example 4

### Butanediol Production

The gene ydjL in Bacillus subtilis encodes acetoin reductase/2,3-butanediol dehydrogenases (AR/BDH), see Nicholson, W. L. 2008. Appl Environ Microbiol. 74(22):6832-6838. AR/BDH produces 2,3 butanediol from acetoin by fermentation.

Taking the B. subtilis sequence to perform a blast search in C. phytofermentans, the closest hit is Cphy_{—}1179 (E-value smaller than 2E-17); same hit as searching for D-glucitol dehydrogenase), which is annotated as alcohol dehydrogenase zinc binding domain.

C. phytofermentans can use acetolactate synthase (either Cphy_{—}3021 or Cphy_{—}3347) to convert pyruvate to 2-acetolactate. Acetolactate decarboxylase converts 2-acetolactate to 2-acetoin, and is found in both B. subtilis and C. acetobutylicum; however, using the sequences from these species, the blast search did not reveal a similar sequence in C. phytofermentans, suggesting C. phytofermentans genome be modified to incorporate this gene.

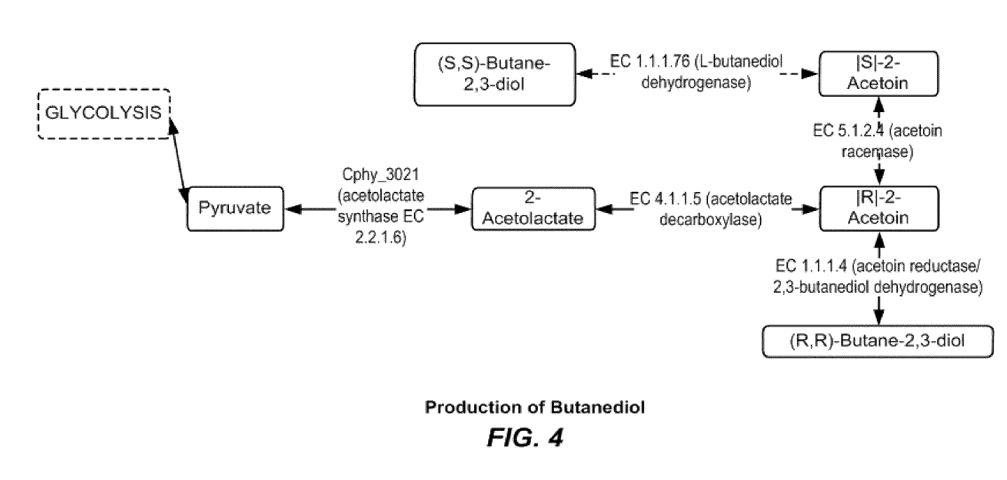

Syu M J. (Appl Microbiol Biotechnol. 2001 January; 55(1):10-8) describes 2,3 butanediol (2,3-BDL) and the metabolic pathway for the microbial formation of 2,3-BDL. Both the biological production of 2,3-BDL and the variety of strains being used were introduced. Genetically improved strains for butanediol (BDL) production which follow either the original mechanisms or new mechanisms were also described and studies on fermentation conditions were also reviewed. Modeling and control of BDL fermentation are discussed. In addition, downstream recovery of 2,3-BDL and the integrated process (being important issues of BDL production) are also introduced (Syu M J. (Appl Microbiol Biotechnol. 2001 January; 55(1):10-8), which is herein incorporated by reference in its entirety). FIG. 4 illustrates the metabolic pathways of C. phytofermentans production of butandiol following incorporation of one or more Bacillus or Clostridium or other microbial genes encoding acetolactate decarboxylase, L-butanediol dehydrogenase, acetoin racemase, and/or acetoin reductase.

### Example 5

### Butanol Production

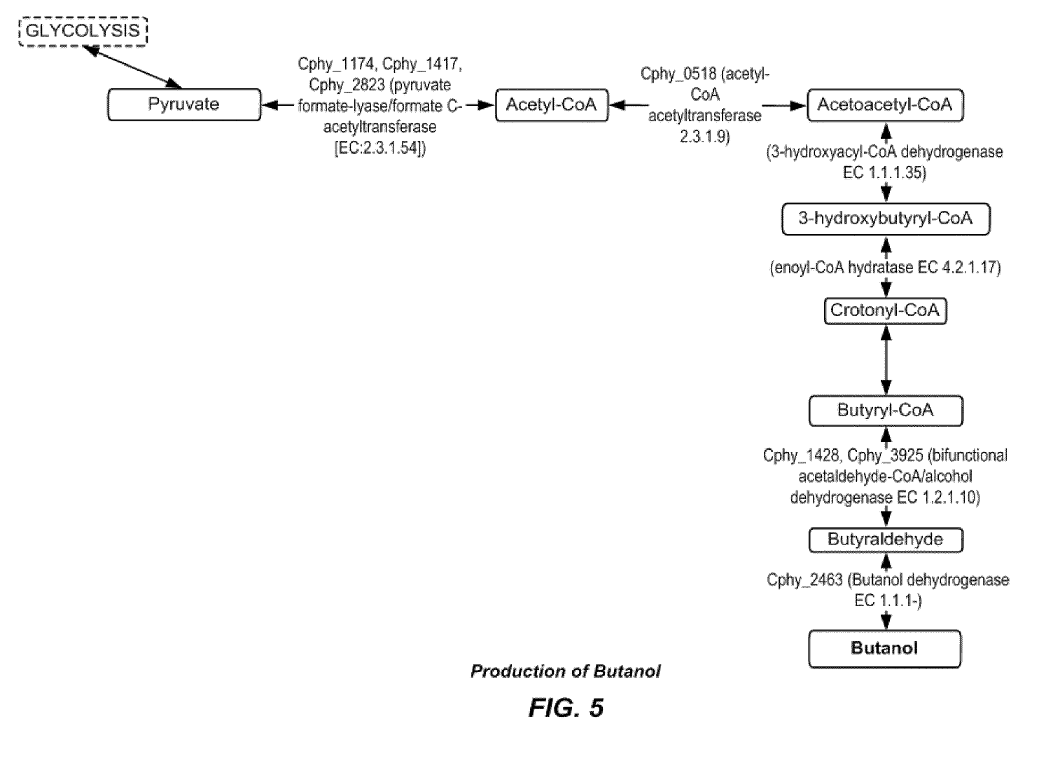

The pathway for Butanol production is well characterized in Clostridium acetobutylicum (Gheshlaghi et al., 2009. Biotechnol Adv, doi:10.1016/j.biotechadv.2009.06.002, and Lee et al., 2008. Fermentative Butanol Production by Clostridia. Biotechnol Bioeng. 101(2):209-28). Components of a butanol synthesis system can comprise enzymes that catalyze reactions, including the conversion of acetyl-CoA to acetoacetyl-CoA; the conversion of acetoacetyl-CoA to 3-hydroxybutyryl-CoA; the conversion of 3-hydroxybutyryl-CoA to crotonyl-CoA; the conversion of crotonyl-CoA to butyryl-CoA; the conversion of butyryl-CoA to butyraldehyde; or the conversion of butyraldehyde to 1-butanol. The enzymes can include acetyl-CoA acetyl transferase, 3-hydroxybutyryl-CoA dehydrogenase, crotonase, butyryl-CoA dehydrogenase, butyraldehyde dehydrogenase, or butanol dehydrogenase. See FIG. 5.

A butanol synthesis system can comprise an enzymatic reaction that converts acetyl-coA to acetoacetyl-CoA. This reaction can be catalyzed by an acetyl-CoA acetyltransferase. A butanol synthesis system can comprise an acetyl-CoA acetyltransferase or variants thereof. An acetyl-CoA acetyltransferase polypeptide can catalyze the conversion of two molecules of acetyl-CoA to acetoacetyl-CoA and coenzyme A (CoA). The acetyl-CoA acetyltransferase can react with substrates short chain acyl-CoA and acetyl-CoA (classified as E.C. 2.3.1.9 (Enzyme Nomenclature 1992, Academic Press, San Diego, which is herein incorporated by reference in its entirety). Enzymes with a broader substrate range (E.C. 2.3.1.16) can also be used. Acetyl-CoA acetyltransferases can be available from a number of sources. A source of Acetyl-CoA acetyltransferase can be E. coli (GenBank Nos: NP-416728; NC-000913); C. acetobutylicum (GenBank Nos: NP-349476.1; NC-003030; NP-149242; NC-001988); B. subtilis (GenBank Nos: NP-390297; NC-000964); or S. cerevisiae (GenBank Nos: NP-015297; NC-001148).

A butanol synthesis system can comprise an enzymatic reaction that converts acetoacetyl-CoA to 3-hydroxybutyryl-CoA. The reaction can be catalyzed by a 3-hydroxybutyryl-CoA dehydrogenase polypeptide. A butanol synthesis system can comprise a 3-hydroxybutyryl-CoA dehydrogenase or variants thereof. 3-hydroxybutyryl-CoA dehydrogenase can catalyze the conversion of acetoacetyl-CoA to 3-hydroxybutyryl-CoA. 3-hydroxybutyryl-CoA dehydrogenases can be reduced nicotinamide adenine dinucleotide (NADH)-dependent, with a substrate preference for (S)-3-hydroxybutyryl-CoA or (R)-3-hydroxybutyryl-CoA and are classified as E. C. 1.1.1.35 and E.C. 1.1.1.30, respectively. Additionally, 3-hydroxybutyryl-CoA dehydrogenases can be reduced nicotinamide adenine dinucleotide phosphate (NADPH)-dependent, with a substrate preference for (S)-3-hydroxybutyryl-CoA or (R)-3-hydroxybutyryl-CoA and are classified as E. C. 1.1.1.157 and E. C. 1.1.1.36, respectively. 3-hydroxybutyryl-CoA dehydrogenases can be from C. acetobutylicum (GenBank NOs: NP-349314), NC-003030), B. subtilis (GenBank NOs: AAB09614, U29084), Ralstonia eutropha (GenBank NOs: YP.sub.—294481, NCJD07347), or Alcaligenes eutrophus (GenBank NOs: AAA21973, J04987).

A butanol synthesis system can comprise an enzymatic reaction that converts 3-Hydroxybutyryl-CoA to Crotonyl-CoA. This reaction can be catalyzed by a crotonase enzyme. A butanol synthesis system can comprise a crotonase or variants thereof. Crotonase can catalyze the conversion of 3-hydroxybutyryl-CoA to crotonyl-CoA and H₂O. Crotonases can have a substrate preference for (S)-3-hydroxybutyryl-CoA or (R)-3-hydroxybutyryl-CoA and are classified as E. C. 4.2.1.17 and E. C. 4.2.1.55, respectively. Crotonases can be from E. coli (GenBank Nos: NP-415911, NC-00913), C. acetobutylicum (GenBank NOs: NP-349318, NC-003030), B. subtilis (GenBank Nos: CAB13705, Z99113), or Aeromonas caviae (GenBank Nos: BAA21816, D88825).

A butanol synthesis system can comprise an enzymatic reaction that converts Crotonyl-CoA to Butyryl-CoA. This reaction can be catalyzed by a butyryl-CoA dehydrogenase enzyme. A butanol synthesis system can comprise a butyryl-CoA dehydrogenase or variants thereof. Butyryl-CoA dehydrogenase can catalyze the conversion of crotonyl-CoA to butyryl-CoA. Butyryl-CoA dehydrogenases can be NADH-dependent or can be NADPH-dependent and can be classified as E.G. 1.3.1.44 and E. C. 1.3.1.38, respectively. Butyryl-CoA dehydrogenases can be available from C. acetobutylicum (GenBank NOs: NP-347102, NC-003030), Euglena gracilis (GenBank NOs: Q5EU90), AY741582), Streptomyces collinus (GenBank NOs: AAA92890, U37135) or Streptomyces coelicolor (GenBank Nos: CAA22721, AL939127).

A butanol synthesis system can comprise an enzymatic reaction that converts Butyryl-CoA to butyraldehyde. This reaction can be catalyzed by a butyraldehyde dehydrogenase enzyme. A butanol synthesis system can comprise a butyraldehyde dehydrogenase or variants thereof. A butyraldehyde dehydrogenase can catalyze the conversion of butyryl-CoA to butyraldehyde, and can use NADH or NADPH as a cofactor. Butyraldehyde dehydrogenases can be from Clostridium beijerinckii (GenBank Nos: AAD31841; AF157306) or C. acetobutylicum (GenBank Nos: NP-149325; NC-001988).

A butanol synthesis system can comprise an enzymatic reaction that converts Butyraldehyde to 1-Butanol. This reaction can be catalyzed by a butanol dehydrogenase enzyme. A butanol synthesis system can comprise a butanol dehydrogenase or variants thereof. A butanol dehydrogenase polypeptide can catalyze the conversion of butyraldehyde to 1-butanol. A butanol dehydrogenase can use NADH or can use NADPH as a cofactor. Butanol dehydrogenases can be from C. acetyobutylicum (GenBank Nos: NP-149325, NC-001988, this enzyme possesses both aldehyde and alcohol dehydrogenase activity); NP-349892, NC-003030) or E. coli (GenBank Nos: NP-417484, NC-000913).

Using the protein sequences from C. acetobutylicum, blast results show that Cphy_{—}0518 (3-oxoacyl-(acyl-carrier-protein) synthase 2) is similar to acetyl-CoA acetyltransferase (E-value smaller than 2E-05), and Cphy_{—}2463 (iron-containing alcohol dehydrogenase) is similar to butanol dehydrogenase (E-value smaller than 1E-125) in C. phytofermentans.

### Example 6

### Itaconic Acid

Itaconate can be produced in C. phytofermentans from a by-product of citrate cycle, cis-Aconitate, by the enzyme cis-aconitate decarboxylase (EC 4.1.1.6). This enzyme is isolated from Aspergillus terreus. Insertion of the gene encoding cis-aconitate decarboxylase from A. terreus and a knockout or reduction of aconitate hydratase in C. phytofermentans allows the production of itaconate in a recombinant C. phytofermentans.

A biosynthesis route(s) for itaconic acid in C. phytofermentans comprise(s) citrate synthase; aconitase; cis-aconitic acid decarboxylase (itaconate-forming); cis-aconitic acid decarboxylase (citraconate-forming); citraconate isomerase; mitochondrial dicarboxylate-tricarboxylate antiporter; mitochondrial tricarboxylate transporter; dicarboxylate transporter; or 2-methylcitrate dehydratase. Production and metabolism of itaconic acid in microbial cells have been studied extensively (Calam, C. T. et al., 1939, Thom. J. Biochem., 33:1488-1495; Bentley, R. and Thiessen, C. P., 1956, J. Biol. Chem. 226:673-720; Cooper, R. A. and Kornberg, H. L., 1964, Biochem. J., 91:82-91; Bonnarme, P. et al., 1995, J. Bacteriol. 117:3573-3578; Dwiarti, L. et al., 2002, J. Biosci. Bioeng. 1:29-33), and Okabe M et al. Appl Microbiol Biotechnol. 2009 September; 84(4):597-606, all of which are herein incorporated by reference in their entirety.

### Example 7

### Glutamic Acid

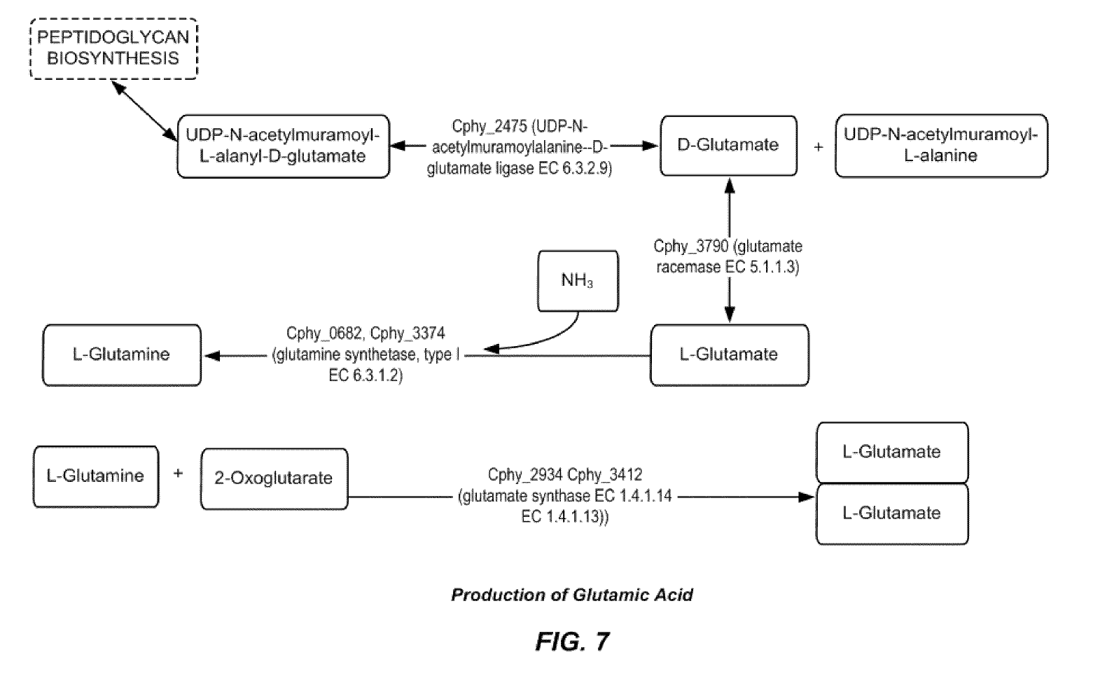

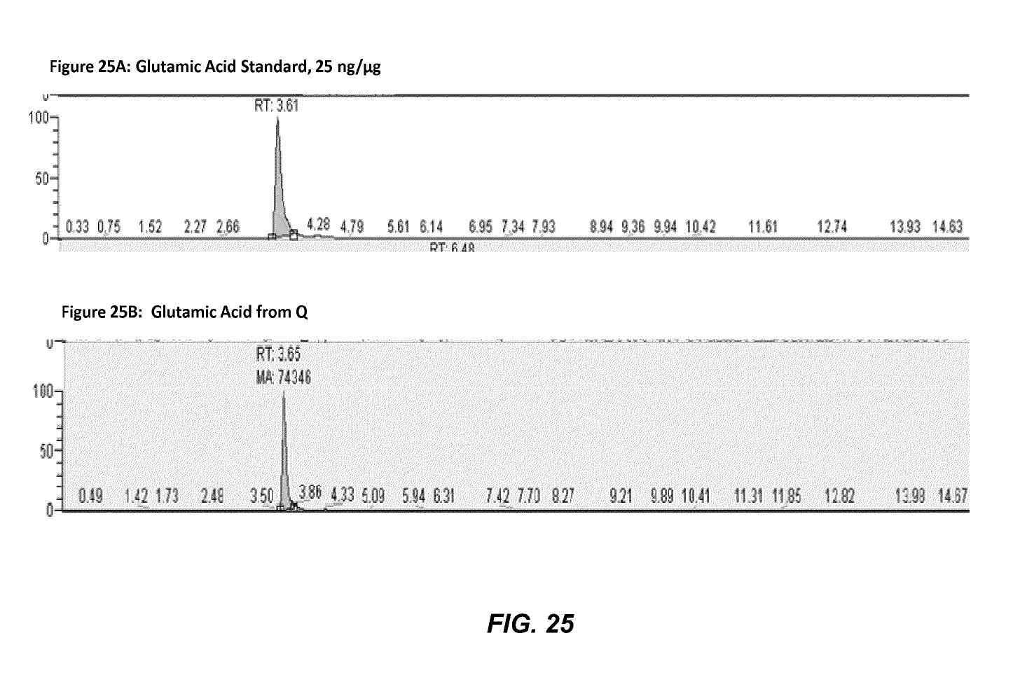

C. phytofermentans naturally synthesizes glutamic acid (FIGS. 7 and 25). D-glutamate is formed by D-glutamate ligase (Cphy_{—}2475), which is converted to L-glutamate by a glutamate racemase (Cphy_{—}3790). L-glutamine is formed by adding an amine group through catalysis by either Cphy_{—}0682 or Cphy_{—}3374 (glutamine synthase). Two molecules of L-glutamate can be formed through combination of a molecule of L-glutamine and a molecule of 2-oxoglutarate by Cphy_{—}2934 or Cphy_{—}3412 (glutamate synthase).

### Example 8

### Glucaric Acid

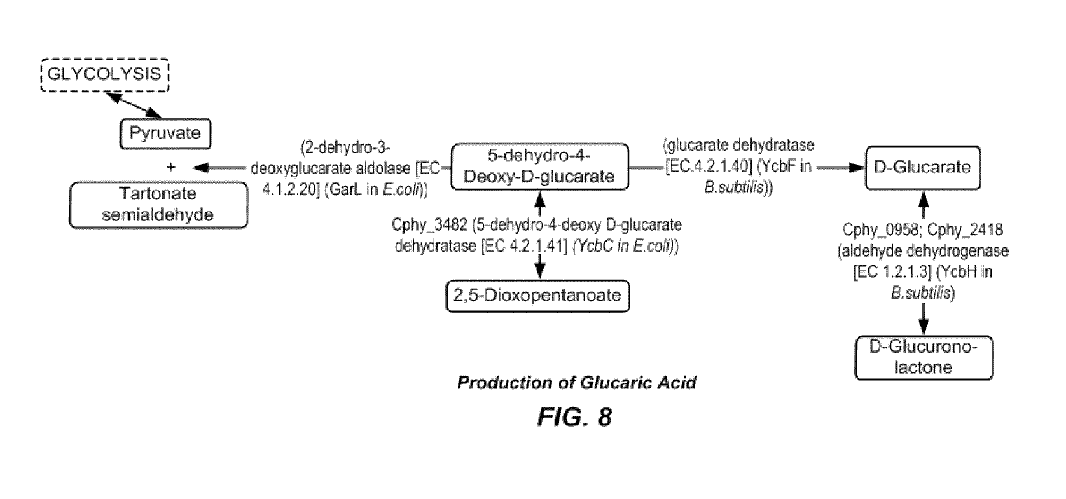

C. phytofermentans does not appear to express glucarate dehydratase. However, the modification of C. phytofermentans to express an exogenous glucarate dehydratase such as YcbF [EC:4.2.1.40] from B. subtilis can allow production of D-glucarate from 5-dehydro-4-deoxy-D-glucarate (FIG. 8). D-glucarate can also be formed from D-glucuronolactone through the Cphy_{—}0958 or Cphy_{—}2418 equivalents of YcbH [EC:1.2.1.3] in B. subtilis. The incorporation of GarL [EC: 4.1.2.20] from E. coli would provide the ability to combine pyruvate and tartonate semialdehyde and produce 5-dehydro-4-deoxy-D-glucarate further enhancing the production of D-glucarate.

### Example 9

### Levulinic Acid Production

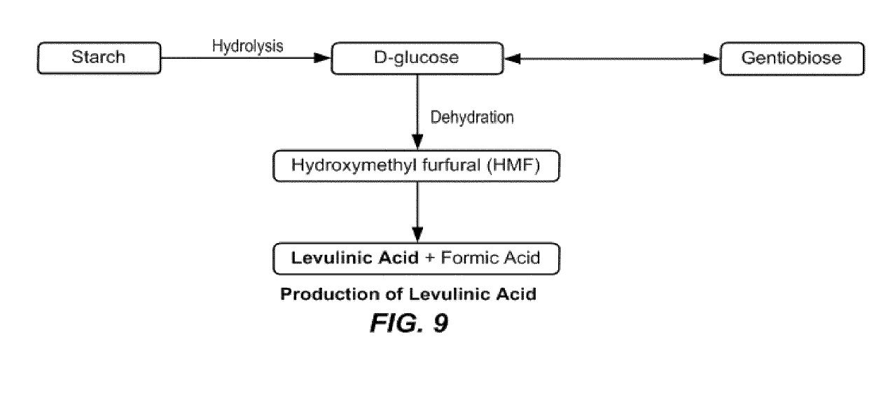

Cha, J. Y. and M. A. Hanna (2002) described levulinic acid production based on extrusion and pressurized batch reaction. Industrial Crops and Products 16(2):109-118, and proposed a reactive extrusion process to hydrolyze starch in the presence of sulfuric acid (5% by weight). The process was able to produce levulinic acid from amylose corn starch at the highest yield of 47.5%. Other sugar-derivatives, e.g. levulose, inulin, starch, and other acids, e.g. sulfuric acids can be substitued for corn starch. A two-step method can be used for the synthesis of levulinic acid from ethyl acetoacetate and ethyl chloroacetate in approximately 70% yield. (FIG. 9)

Jeong, G.-T. and D.-H. Park (2009) Production of Sugars and Levulinic Acid from Marine Biomass Gelidium amansii. Appl Biochem Biotechnol., doi:10.1007/s12010-009-8795-5, also provides a method to produce levulinic acid from marine algal biomass Gelidium amansii. Both methods produce levulinic acid through a chemical route, and either can be used for the biosynthesis of this compound from microorganisms, such as C. phytophermentans or C. sp. Q.D.

### Example 10

### 2,5-Furandicarboxylic Acid Production

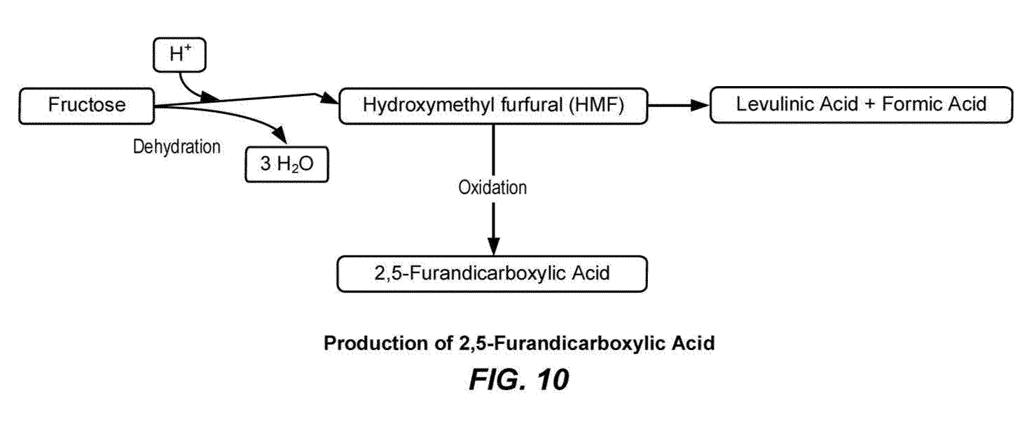

Kröger et al. (2000) Topics in Catalysis. 13: 237-242, demonstrated a two-phase system approach for the production of 2,5-furandicarboxylic acid by in situ oxidation of 5-hydroxymethylfurfural starting from fructose. The production of hydroxymethylfurfural (HMF) is carried out in the water phase, and the oxidation reaction in methyl isobutyl ketone. This in situ oxidation gives a 25% yield of 2,5-furandicarboxylic acid. (FIG. 10) Production of 2,5-furandicarboxylic acid (FDCA) can start with fructose as substrate via acid-catalyzed formation and subsequent oxidation of 5-hydroxymethylfurfural (HMF). For example, preparations of 5-hydroymethylfurfural (HMF) by the dehydration of fructose can be carried out in the presence of the Brønsted acidic ionic liquid, 3-allyl-1-(4-sulfobutyl)imidazolium trifluoromethanesulfonate, as well as its Lewis acid derivative, 3-allyl-1-(4-sulfurylchloride butyl)imidazolium trifluoromethanesulfonate. An effective separation of the oxidation catalyst from fructose in combination with extraction and derivatization of formed HMF in methyl isobutyl ketone (MIBK) leads to formation of FDCA as final product. For example, HMF is preferentially oxidized by dioxygen and metal/bromide catalysts [Co/Mn/Br, Co/Mn/Zr/Br; Co/Mn=Br/(Co+Mn) to form the dialdehyde, 2,5-diformylfuran (DFF). HMF can be also oxidized, via a network of identified intermediates, to the highly insoluble 2,5-furandicarboxylic acid.

Ribeiro, M. L. and U. Schuchardt ((2003) Cooperative effect of cobalt acetylacetonate and silica in the catalytic cyclization and oxidation of fructose to 2,5-furandicarboxylic acid. Catalysis Communications. 4(2):83-86), suggested the one-pot conversion of fructose to 2,5-furandicarboxylic acid by using cobalt acetylacetonate encapsulated in a sol-gel silica matrix. The system converted 72% of fructose to 2,5-furandicarboxylic acid with 99% selectivity, thus there is little formation of by-product.

Biosynthesis of this compound from C. phytofermentans is initiated by the dehydration of fructose produced from fermentation. The HMF produced is oxidated to 2,5-furandicarboxylic acid.

### Example 11

### Aspartic Acid Production

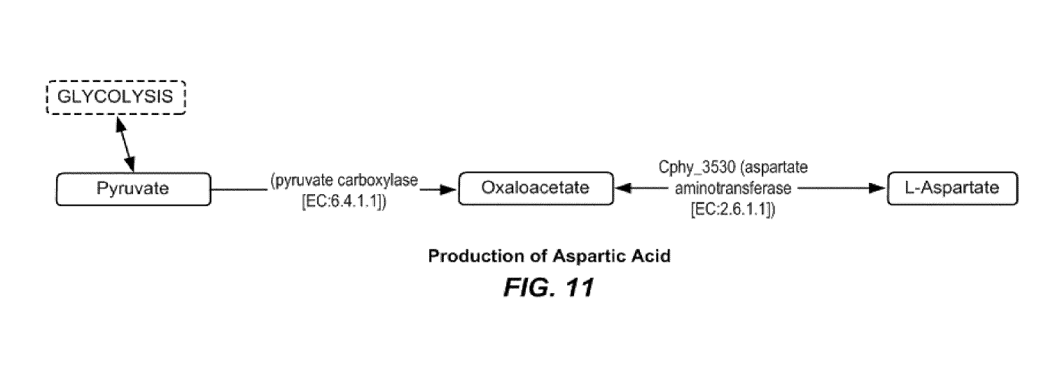

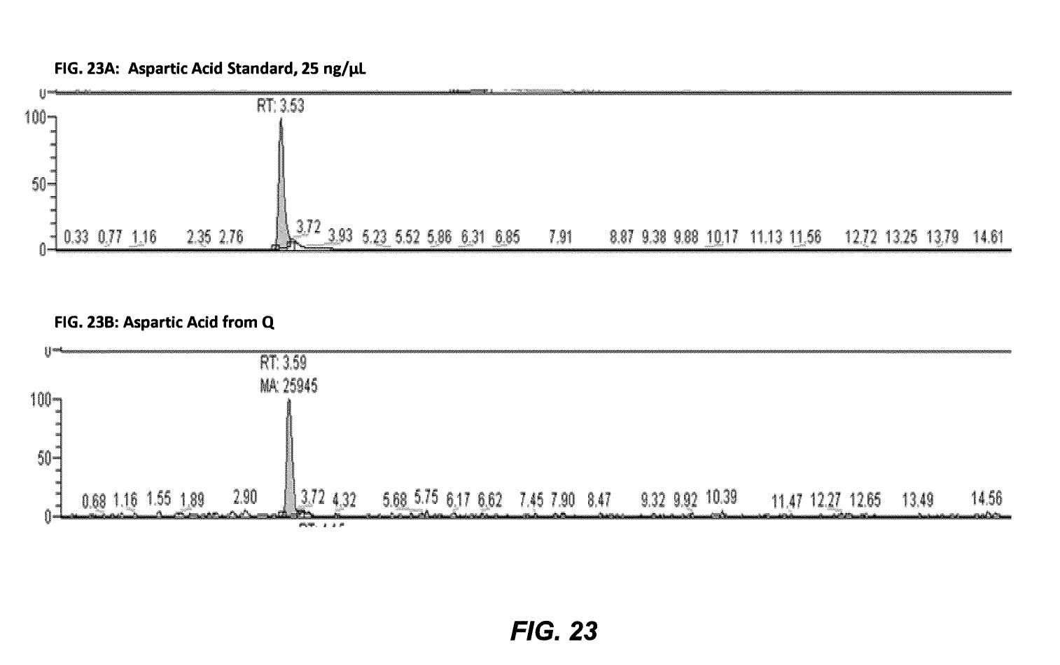

C. phytofermentnans naturally synthesizes aspartic acid (FIG. 23). Aspartic acid can be made from oxaloacetate by conversion of the oxaloacetate to aspartate by a transaminase enzyme. The transaminase enzyme transfers the amino group from glutamate to oxaloacetate producing α-ketoglutarate and aspartate. The enzyme asparagine synthetase produces asparagine, AMP, glutamate, and pyrophosphate from aspartate, glutamine, and ATP. In the asparagine synthetase reaction, ATP is used to activate aspartate, forming β-aspartyl-AMP. Glutamine donates an ammonium group which reacts with β-aspartyl-AMP to form asparagine and free AMP. Clostridium phytofermentans gene Cphy_{—}3530 expresses aspartate aminotransferase for conversion of oxaloacetate to L-Aspartate (FIG. 11); but DNA for the expression of pyruvate carboxylase must be introduced. Incorporation of the Bacillus subtilis gene for pyruvate carboxylase, pycA completes the pathway for the production of aspartic acid in Clostridium.

In addition, aspartate dehydrogenase catalyses synthesis of aspartate from oxaloacetic acid and ammonia in the presence of NADH. Aspartase (L-aspartate ammonialyase) catalyses synthesis of aspartate from fumaric acid and ammonia.

### Example 12

### 1,4 Diacid Production (Succinic Acid, Fumaric Acid, and Malic Acid) Production

Dicarboxylic acids are organic compounds that are substituted with two carboxylic acid functional groups. In molecular formulae for dicarboxylic acids, these groups are often written as HOOC—R—COOH, where R is usually an alkyl, alkenyl, or akynyl group.

Succinic acid, fumaric acid and malic acid are 4-carbon compounds that have two carboxyl groups and are termed dicarboxylic acid (diacid).

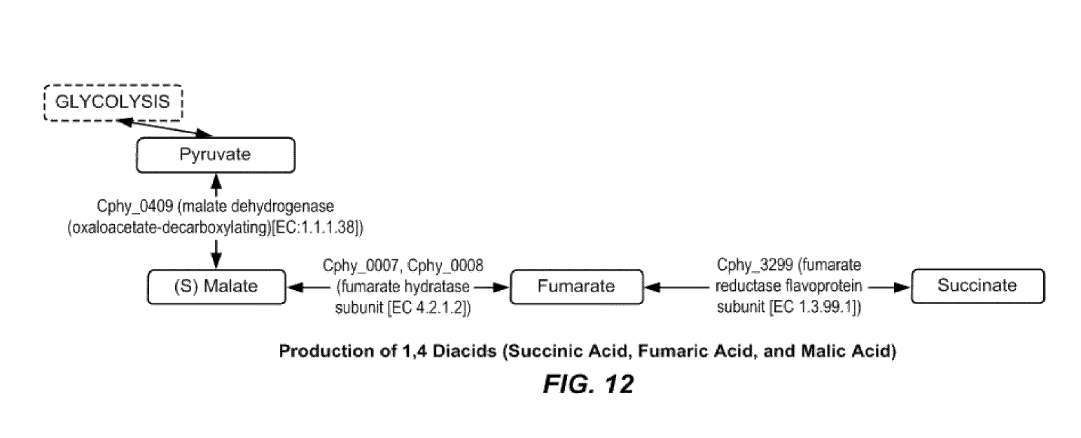

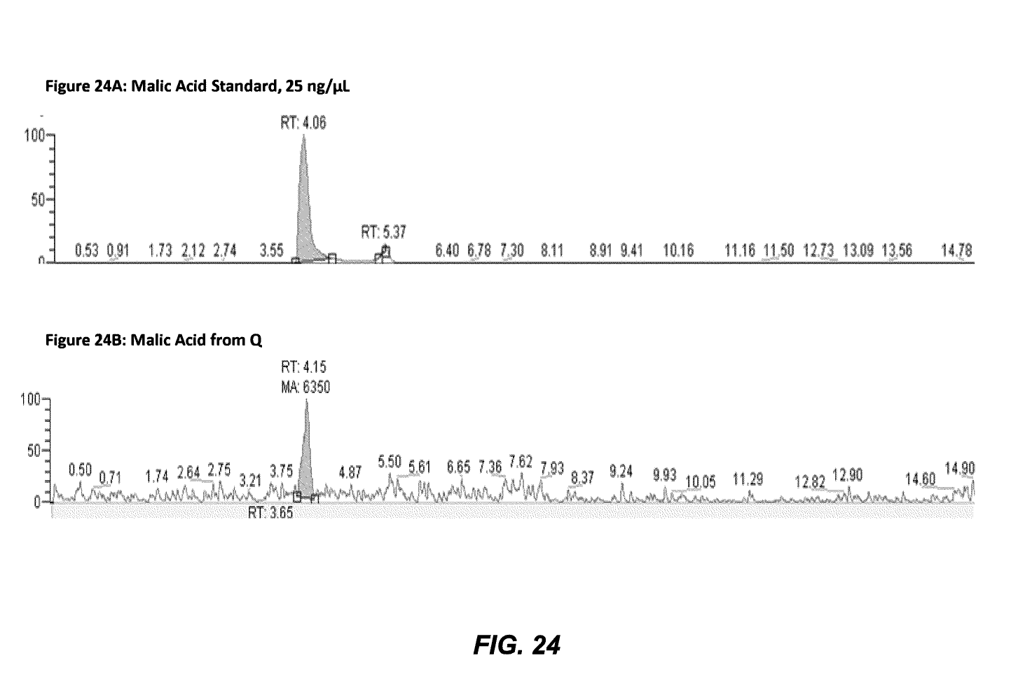

Clostridium phytofermentans can produce fumaric, succinic and malic acids without incorporating heterologous genes or through insertion of additional copies of its own genes under the control of appropriate promoters and regulatory elements. As part of its genome, C. phytofermentans comprises the gene Cphy_{—}0409 for converting pyruvate to L-malic acid, the genes Cphy_{—}0007, Cphy_{—}0008 for converting L-malic acid to fumaric acid, and Cphy_{—}3299 or Cphy_{—}3885 for converting fumaric acid to succinic acid (FIG. 12). Natural yields of malic acid are shown in FIG. 24. Production of L-malic acid involves conversion of pyruvic acid to oxaloacetic acid by pyruvate carboxylase, followed by conversion of oxaloacetic acid to L-malic acid. Bioconversion of fumaric acid to succinic acid by recombinant E. coli has been described in Applied Biochemistry and Biotechnology Volume 70-72, Number 1/March, 1998, which is herein incorporated by reference in its entirety. The metabolic pathways leading to the synthesis of succinic acid, fumaric acid and malic acid and their inter-conversions in bacteria fermentation are reviewed by Hyohak Song and Sang Yup Lee Enzyme and Microbial Technology Volume 39, Issue 3, 3 Jul. 2006, Pages 352-361, which is herein incorporated by reference in its entirety.

### Example 13

### 3-Hydroxy Propionic Acid Production

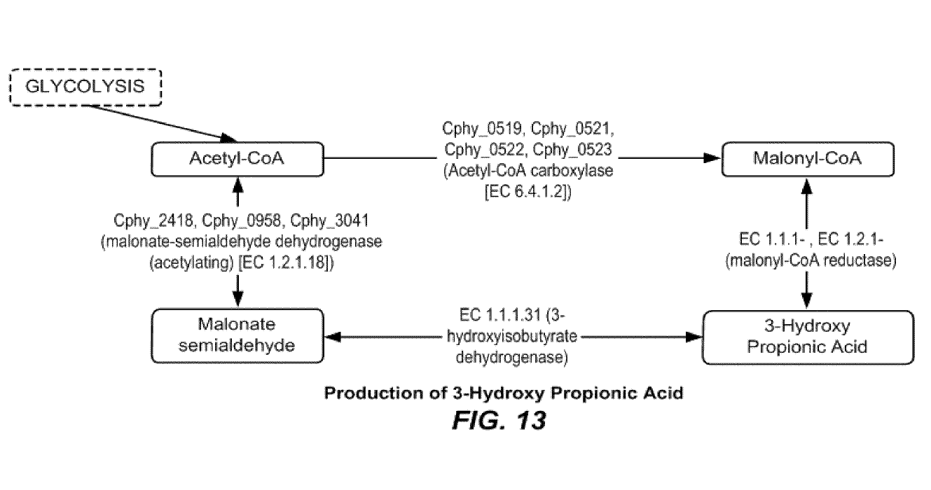

3-Hydroxy propionic acid (3-HP) can be conventionally produced by the hydration of acrylic acid or by the conversion of ethylene chlorohydrin with sodium cyanide (Ullman's Encyclopedia of Industrial Chemistry, 5th Edition, Volume A-13, Pages 507 to 517, which is herein incorporated by reference in its entirety). 3-hydroxy propionic acid can be naturally produced from acetyl-CoA through either malonyl-CoA or malonate semialdehyde in C. phytofermentans (FIG. 13). The 3-HP synthesis pathway involves CO₂-, MgATP-, and NADPH-dependent conversion of acetyl-CoA to 3-hydroxypropionate via malonyl-CoA and the conversion of this intermediate to succinate via propionyl-CoA. The biosynthetic pathways for 3-hydroxypropionic acid production are reviewed by Jiang X et al. Appl Microbiol Biotechnol. 2009 April; 82(6):995-1003, which is herein incorporated by reference in its entirety.

Bradyrhizobium japonicum expresses malonate-semialdehyde dehydrogenase activities for conversion of acetyl-CoA to malonate semialdehyde. Using the DNA sequence of the Bradyrhizobium japonicum gene for this enzyme to perform a blast search in Clostridium phytofermentans, the closest hits are the three aldehyde dehydrogenase, Cphy_{—}2418, Cphy_{—}0958, and Cphy_{—}3041 (E-value smaller than 8E-08).

Several microorganisms have genes that can be expressed in Clostridium sp. to complete the pathway to 3-hydroxypropionic acid. Xian et al. (2009) (Biosynthetic pathways for 3-hydroxypropionic acid production. Appl Microbiol Biotechnol. 82:995-1003.) noted that Pseudomona aeruginosa expresses 3-hydroxyisobutyrate dehydrogenase (EC 1.1.1.31) for conversion of malonate semialdehyde to 3-hydroxy propionic acid. Xian et al. (2009) also indicated that Chloroflexus aurantiacus contains the gene for malonyl-CoA reductase to produce 3-hydroxy propionic acid.

Also disclosed are methods of producing 3-hydroxypropionic acid (3-HP) from beta-alanine through a malonate semialdehyde intermediate using beta-alanine/pyruvate aminotransferase. 3-hydroxypropionic acid can be produced by biocatalysis from beta-alanine. Beta-alanine can be synthesized in cells from carnosine, beta-alanyl arginine, beta-alanyl lysine, uracil via 5,6-dihydrouracil and N-carbamoyl-beta-alanine, N-acetyl-beta-alanine, anserine, or aspartate. Beta-alanine can also be produced from alpha-alanine by an enzyme having alanine 2,3-aminomutase activity.

### Example 14

### Synthesis of Terpenes and Terpenoids (or Isoprenoids)

The terpenoids sometimes called isoprenoids, are a large and diverse class of naturally-occurring organic chemicals (similar to terpenes) derived from five-carbon isoprene units. These lipids can be found in all classes of living things, and are the largest group of natural products.

Isoprene (short for isoterpene), or 2-methyl-1,3-butadiene (C₅H₈), is an organic compound with the formula CH₂═C(CH₃)CH═CH₂ and is the monomer of natural rubber. Molecular formulas of isoprenoids are stated as multiples of isoprene in the form of (C₅H₈)ₙ (the isoprene rule). The basic functional isoprene units in biological systems are dimethylallyl pyrophosphate (DMAPP) and its isomer isopentenyl pyrophosphate (IPP).

The singular terms “isoprene” and “terpene” are synonymous whereas the plurals “isoprenes” or “terpenes” refer to terpenoids (isoprenoids). Terpenes are hydrocarbons resulting from the combination of several isoprene units. Terpenoids can be thought of as modified terpenes, wherein methyl groups have been moved or removed, or oxygen atoms added.

Prokaryotes (with some exceptions), use the non-mevalonate pathway or 2-C-methyl-D-erythritol 4-phosphate/1-deoxy-D-xylulose 5-phosphate pathway (MEP/DOXP pathway) to generate the required terpenoid structures essential for replication (e.g. prenylated tRNAs) or cell membrane composition and integrity (e.g. polyprenoids) or solvent tolerance (e.g. hopanoids).

In this pathway, pyruvate and glyceraldehyde 3-phosphate are converted by DOXP synthase (Dxs) to 1-deoxy-D-xylulose 5-phosphate, and by DOXP reductase (Dxr, IspC) to 2-C-methyl-D-erythritol 4-phosphate (MEP). The next three reaction steps catalyzed by 4-diphosphocytidyl-2-C-methyl-D-erythritol synthase (YgbP, IspD), 4-diphosphocytidyl-2-C-methyl-D-erythritol kinase (YchB, IspE), and 2-C-methyl-D-erythritol 2,4-cyclodiphosphate synthase (YgbB, IspF) mediate the formation of 2-C-methyl-D-erythritol 2,4-cyclopyrophosphate (MEcPP).

Finally, MEcPP is converted to (E)-4-hydroxy-3-methyl-but-2-enyl pyrophosphate (HMB-PP) by HMB-PP synthase (GcpE, IspG), and HMB-PP is converted to isopentenyl pyrophosphate (IPP) and dimethylallyl pyrophosphate (DMAPP) by HMB-PP reductase (LytB, IspH).

IPP and DMAPP are the precursors of isoprene, monoterpenoids (10-carbon), diterpenoids (20-carbon), carotenoids (40-carbon), chlorophylls, and plastoquinone-9 (45-carbon). Synthesis of all higher terpenoids proceeds via formation of geranyl pyrophosphate (GPP), farnesyl pyrophosphate (FPP), and geranylgeranyl pyrophosphate (GGPP).

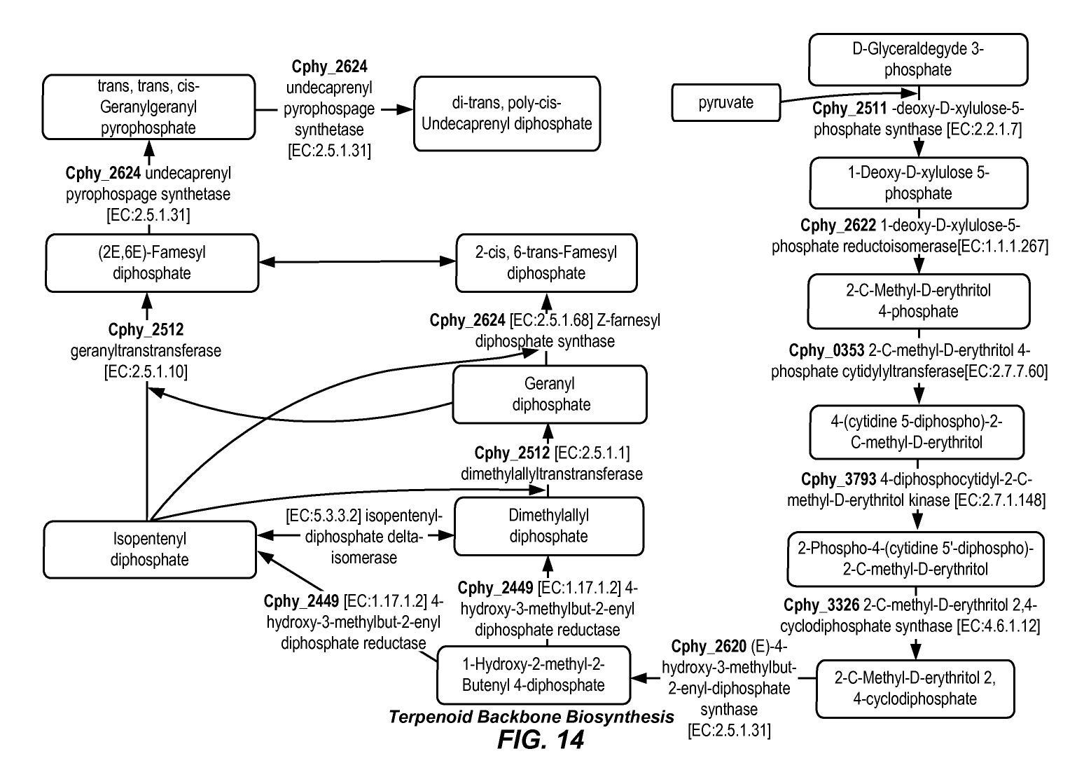

FIG. 14 illustrates the terpenoid backbone systhesis in C. phytofermentans, providing the metabolic pathways wherein GPP and IPP can be synthesized in Clostridia using overexpression of naturally-occurring genes and also through the expression of exogenous DNA. In some instances, e.g., to make rubber, IPP would be diverted and polymerized to rubber while the conversion of IPP to farnesyl diphosphate was inhibited. Regulation of promoters would ensure that enough farnesyl diphosphate was synthesized to maintain the function of the cell.

### Example 15

### Mass Spectrophotometer Analysis

Materials

All chemicals were from Sigma-Aldrich (St. Louis, Mo.), unless otherwise noted. Clostridium phytofermentans strain Q.8 was grown in Basal medium (see infra). Conical tubes and microcentrifuge tubes were from VWR (Radnor, Pa.). Centrifuge was from Eppendorf (Hauppauge, N.Y.), model 5810R. Microcentrifuge was from Beckman Coulter (Brea, Calif.), model A46474.

Basal Medium

Add to 700 g of ddH₂O: 3.0 g K₂HPO₄, 1.6 g KH₂PO₄, 2.5 g Bacto Yeast Extract, 1.0 g NaCl, 2.0 g (NH₄)₂50₄. Adjust pH of solution to 7.5 with NaOH, add ddH₂O to 940 g, then autoclave and add filter-sterilized: 100× amino Acid Stock and 100×B Vitamin Stock. (See Tables 3 and 4.)

TABLE 3

| | Gram per |
|---|---|
| 100X salt components | Liter |
| Na₃C₆H₅O₇ | 10 |
| CaCl₂•2H₂O | 0.5 |
| MgSO₄•7H₂O | 6 |
| FeSO₄•7H₂O | 0.4 |
| CoSO₄•H₂O | 0.2 |
| ZnSO₄•7H₂O | 0.2 |
| NiCl₂ | 0.2 |
| MnSO₄•H₂O | 0.5 |
| CuSO₄•5H₂O | 0.04 |
| KAl(SO₄)₂•12H₂O | 0.04 |
| H₃BO₃ | 0.04 |
| (NH4)6Mo7O24•4H2O | 0.04 |
| Na2SeO3 | 0.04 |

Each salt is added in order (allowing each to dissolve prior to next addition) to 1000 g ddH₂O and mixed.

TABLE 4

| 100X vitamin | Gram per |
|---|---|
| components | Liter |
| dd H₂O | 1000 |
| Pantethine | 0.5 |
| Nicotinic acid | 1.5 |
| Pyridoxine HCL | 0.5 |
| Cyanocobalamine | 0.5 |
| Thiamine | 0.5 |
| Riboflavin | 0.5 |
| Folinic Acid | 0.015 |

Add each vitamin one at a time and allow to dissolve before next addition. Add riboflavin last with one pellet of NaOH until dissolved; remove pellet.

A stock culture of Q.8 was innoculated into fresh medium and grown to an OD660 of about 0.710. Ten mL aliquots were transferred into each of four 50 ml conical tubes. Thirty mL of pre-chilled (30 min at −80° C.) 60% methanol solution (methanol from J. T. Baker, Phillipsburg, N.J., diluted in dH₂O) was added to each tube, and tubes gently inverted to mix. Tubes were centrifuged at 8500 rpm for 10 min at 4° C. The supernatant was decanted and the methanol wash repeated using 10 mL methanol solution per tube. Following centrifugation, the supernatant was decanted, and the pellets flash frozen on an ethanol dry ice mix. The tubes were then placed at −80° C. for storage. In the second set, after last centrifugation, the pellets were resuspended in 1 mL of methanol, and then transferred to 1.5 mL micro centrifuge tubes and centrifuged to obtain final pellet. These samples were stored at −80° C.

Sample Extraction

To samples delivered in 50 mL conical tubes, one mL of cold (−80° C.) methanol was added to suspend the pellet. Tube was vortex mixed for one minute, and centrifuged (HF-120 centrifuge) for three minutes.

To samples delivered in 1.5 mL conical tubes, one hundred μL of cold (−80° C.) methanol was added to suspend the pellet. Tube was mixed by vortex mixer for one minute, and centrifuged for three minutes.

Sample Analysis

LC/MS/MS—for Identification of Aspartic Acid, Malic Acid, and Glutamic Acid.

An HPLC system (Accela pump with PAL autosampler, ThermoFisher, Waltham, Mass.) equipped with a TSQ Quantum Ultra triple-quad mass spectrometer (MS) (ThermoFisher) was used. Separation was achieved on an Eclipse XDB-C18 (Agilent, Palo Alto, Calif.) column, DIMENSIONS. Mobile phasea were A) H₂O/0.1% formic acid and B) acetonitrile/0.1% formic acid, respectively. The gradient used was 0-100% B in 10 minutes, hold for 2 minutes and equilibrate for 3 minutes, for a total 15 minute run time. MS conditions were as follows: Negative mode multiple reaction monitoring (MRM); electrospray voltage=3000; sheath gas pressure=25; capillary temp=290° C. Parent Ion/Daughter Ions monitored were as follows: 132.100/88.068 aspartic acid; 133.100/115.002 malic acid; and 146.130/128.099 glutamic acid.

Standard solutions at a concentration of 25 ng/μg were prepared in methanol of each of the following: DL-malic acid, 99%; L-glutamic acid, 99%; L-aspartic acid, 99% lithium salt, 90%. Standards were injected and the listed parent/daughter ions were monitored. Samples were injected and response was compared to standards. Results are shown in FIGS. 23-25.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

What is claimed is:

1. A composition for producing first and second fermentation end-products comprising:
a. a carbonaceous biomass;
b. a microorganism that hydrolyses and ferments pentose and hexose saccharides from said biomass;
c. a first fermentation end-product, wherein said first fermentation end-product is not ethanol, propanol, isopropanol, n-butanol, hydrogen, formic acid, lactic acid, acetic acid, formate, lactate or acetate; and
d. a second fermentation end-product.

2. (canceled)

3. The composition of claim 1, wherein said microorganism is genetically modified to produce a higher concentration of said first fermentation end-product compared to a non-genetically modified form of said microorganism.

4. (canceled)

5. The composition of claim 1, wherein said microorganism is a bacterium.

6. The composition of claim 1, wherein said microorganism is a species of Clostridia.

7. The composition of claim 1, wherein said microorganism is Clostridium phytofermentans.

8. The composition of claim 1, wherein said microorganism is Clostridium sp. Q.D.

9. The composition of claim 1, wherein the microorganism is non-recombinant.

10. The composition of claim 1, wherein the microorganism is recombinant.

11. The composition of claim 1, wherein said microorganism is genetically modified to enhance production of a fermentation end-product.

12. The composition of claim 1, wherein said microorganism is genetically modified to express a protein encoded by a heterologous polynucleotide.

13. The composition of claim 1, wherein said microorganism is genetically modified to enhance expression of a protein by deregulation of an endogenous promoter or by expression of an additional copy of an endogenous polynucleotide encoding said protein.

14. (canceled)

15. The composition of claim 1, wherein said first fermentation end-product is aspartic acid, aspartate, glutamic acid, glutamate, malic acid, or malate.

16. (canceled)

17. The composition of claim 1, wherein said second fermentation end-product is not ethanol, propanol, isopropanol, n-butanol, hydrogen, formic acid, lactic acid, acetic acid, formate, lactate or acetate.

18. The composition of claim 1, wherein said second fermentation end-product is ethanol, propanol, isopropanol, n-butanol, hydrogen, formic acid, lactic acid, acetic acid, formate, lactate or acetate and is present at a lower amount than said first fermentation product.

19. The composition of claim 1, wherein said second fermentation end-product is ethanol, propanol, isopropanol, n-butanol, hydrogen, formic acid, lactic acid, acetic acid, formate, lactate or acetate and is present at an amount less than 99%, 75%, 50%, 30%, 20%, or 10% of the amount of said first fermentation product.

20-26. (canceled)

27. The composition of claim 1, wherein said first fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene.

28. (canceled)

29. The composition of claim 1, wherein said carbonaceous biomass comprises one or more of xylan, cellulose, hemicellulose, fructose, glucose, mannose, rhamnose, or xylose.

30. The composition of claim 1, wherein said carbonaceous biomass is plant matter.

31. The composition of claim 1, wherein said carbonaceous biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae.

32. The composition of claim 1, wherein said carbonaceous biomass comprises a higher concentration of oligomeric carbohydrates relative to monomeric carbohydrates.

33. The composition of claim 1, wherein said hydrolysis provides results in a greater concentration of cellobiose relative to monomeric carbohydrates.

34. The composition of claim 32, wherein said monomeric carbohydrates comprise xylose and arabinose.

35. The composition of claim 1, wherein said carbonaceous biomass is pre-treated with an acid, alkali or heat prior to contact with said microorganism.

36. The composition of claim 1, further comprising a second species of microorganism.

37. The composition of claim 36, wherein said second species of microorganism is selected from the group consisting of a yeast, an other fungus, and a bacterium.

38. The composition of claim 35, wherein said second species of microorganism is S. cerevisiae or Aspergillus niger.

39. A fermentation composition comprising:
a. a carbonaceous biomass;
b. a strain of Clostridium phytofermentans; and
c. a fermentation end-product comprising aspartic acid, aspartate, glutamic acid, glutamate, malic acid, or malate,
wherein said strain of Clostridium phytofermentans produces said fermentation end-product from said carbonaceous biomass.

40. (canceled)

41. A process for producing a first and second fermentation end-product comprising:
a. contacting a carbonaceous biomass with: a microorganism that hydrolyses and ferments pentose and hexose saccharides from said carbonaceous biomass to produce said first and second fermentation end-products, wherein said first fermentation end-product is not ethanol, propanol, isopropanol, n-butanol, hydrogen, formic acid, lactic acid, acetic acid, formate, lactate or acetate; and
b. allowing sufficient time for said hydrolysis and fermentation to produce said first and second fermentation end-products.

42. The process of claim 41, wherein said microorganism is genetically modified to produce a higher concentration of said first fermentation end-product compared to a non-genetically modified or non-mutagenized form of said microorganism.

43. (canceled)

44. The process of claim 41, wherein said microorganism is a bacterium.

45. The process of claim 41, wherein said microorganism is a species of Clostridia.

46. The process of claim 41, wherein said microorganism is Clostridium phytofermentans.

47. The process of claim 41, wherein said microorganism is Clostridium sp. Q.D.

48. (canceled)

49. (canceled)

50. The process of claim 41, wherein said microorganism is genetically modified to enhance production of a fermentation end-product.

51-53. (canceled)

54. The process of claim 41, wherein said first fermentation end-product is aspartic acid, aspartate, glutamic acid, glutamate, malic acid, or malate.

55. (canceled)

56. The process of claim 41, wherein said first fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene.

57. (canceled)

58. (canceled)

59. The process of claim 41, wherein said second fermentation end-product is not ethanol, propanol, isopropanol, n-butanol, hydrogen, formic acid, lactic acid, acetic acid, formate, lactate or acetate.

60. The process of claim 41, wherein said second fermentation end-product is ethanol, propanol, isopropanol, n-butanol, hydrogen, formic acid, lactic acid, acetic acid, formate, lactate or acetate and is present at a lower amount than said first fermentation product.

61-105. (canceled)

106. A system for producing a fermentation end-product comprising a fermentation vessel comprising:
a. a carbonaceous biomass; and
b. a microorganism that hydrolyses and ferments pentose and hexose saccharides from said carbonaceous biomass, wherein said microorganism produces a first and second fermentation end-products, wherein said first fermentation end-product is not ethanol, propanol, isopropanol, n-butanol, hydrogen, formic acid, lactic acid, acetic acid, formate, lactate or acetate, wherein said fermentation vessel is adapted to provide suitable conditions for fermentation of pentose and hexose saccharides into said first and second fermentation end-products.

107. (canceled)

108. The system of claim 106, wherein said microorganism is genetically modified to produce a higher concentration of said first fermentation end-product compared to a non genetically modified or mutagenized form of said microorganism, wherein said fermentation vessel is adapted to provide suitable conditions for fermentation of pentose and hexose saccharides into said fermentation end-product.

109. (canceled)

110. The system of claim 106, wherein said microorganism is a bacterium.

111. The system of claim 106, wherein said microorganism is a species of Clostridia.

112. The system of claim 106, wherein said microorganism is Clostridium phytofermentans.

113. The system of claim 106, wherein said microorganism is Clostridium sp. Q.D.

114-120. (canceled)

121. The system of claim 106, wherein said first fermentation end-product is aspartic acid, aspartate, glutamic acid, glutamate, malic acid, or malate.

122. (canceled)

123. (canceled)

124. The system of claim 106, wherein said first fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene.

125. The system of claim 106, wherein said second fermentation end-product is not ethanol, propanol, isopropanol, n-butanol, hydrogen, formic acid, lactic acid, acetic acid, formate, lactate or acetate.

126. The system of claim 106, wherein said second fermentation end-product is ethanol, propanol, isopropanol, n-butanol, hydrogen, formic acid, lactic acid, acetic acid, formate, lactate or acetate and is present at a lower amount than said first fermentation product.

127-143. (canceled)

144. A composition for producing first and second fermentation end-products comprising:
a. a carbonaceous biomass;
b. a Clostridium phytofermentans strain that hydrolyses and ferments pentose and hexose saccharides from said biomass;
c. a first fermentation end-product, wherein said first fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene; and
d. a second fermentation end-product, wherein said second fermentation end-product is not ethanol, propanol, isopropanol, n-butanol, hydrogen, formic acid, lactic acid, acetic acid, formate, lactate or acetate.

145. A composition for producing first and second fermentation end-products comprising:
a. a carbonaceous biomass;
b. a Clostridium phytofermentans strain that hydrolyses and ferments pentose and hexose saccharides from said biomass;
c. a first fermentation end-product, wherein said first fermentation end-product is a 1,4 diacid (succinic, fumaric or malic), 2,5 furan dicarboxylic acid, 3 hydroxy propionic acid, aspartic acid, aspartate, glucaric acid, glutamic acid, glutamate, malate, itaconic acid, levulinic acid, 3-hydroxybutyrolactone, glycerol, sorbitol, xylitol/arabinitol, butanediol, an isoprenoid, or a terpene; and
d. a second fermentation end-product, wherein said second fermentation end-product is ethanol, propanol, isopropanol, n-butanol, hydrogen, formic acid, lactic acid, acetic acid, formate, lactate or acetate and is present at a lower amount than said first fermentation product.

146. A fermentation end-product produced by the process of claim 41, wherein said microorganism is Clostridium phytofermentans or mutant thereof.

147. The fermentation end-product of claim 146, wherein said fermentation end-product is aspartic acid, aspartate, glutamic acid, glutamate, malic acid, or malate.
